(19) United States
(12) Patent Application Publication
(10) Pub. No.: US 2025/0127894 A1
(43) Pub. Date: Apr. 24, 2025

(54) PROCESS FOR PRODUCING ARMED IMMUNE CELLS

(71) Applicant: CYTOARM CO., LTD., Taipei City (TW)

(72) Inventors: Michael Chen, Taipei City (TW); Kuo-Hsiang Chuang, Taipei City (TW); Yi-Jou Chen, Taipei City (TW)

(21) Appl. No.: 18/682,632

(22) PCT Filed: Aug. 10, 2022
(86) PCT No.: PCT/US2022/074776

§ 371 Date: Feb. 9, 2024

Related U.S. Application Data
(60) Provisional application No. 63/231,934, filed on Aug. 11, 2021.

(51) Int. Cl. A61K 40/11; A61K 40/42; A61K 40/50; C07K 16/28; C12N 5/0783

(52) U.S. Cl. CPC A61K 40/11; A61K 40/421; A61K 40/4211; A61K 40/50; C07K 16/2803; C07K 16/2809; C12N 5/0636; C07K 2317/31; C07K 2317/622; C07K 2317/732

(57) ABSTRACT

Method for preparing armed immune cells, comprising: isolating a population of CD3⁺ immune cells from a human blood sample; and contacting the population of immune cells with a bi-specific antibody specific to CD3 and a tumor associated antigen (TAA) to produce armed immune cells, which display the bi-specific antibody on the cell surface.

### CROSS REFERENCE TO RELATED APPLICATIONS

This application claims the benefit of the filing dates of U.S. Provisional Application No. 63/231,934, filed Aug. 11, 2021, the entire contents of which are incorporated by reference herein.

### BACKGROUND OF THE INVENTION

Cancer is a disease characterized by abnormal cells that divide uncontrollably and have the ability to infiltrate and destroy normal tissue and/or organ of a subject. Cancer is the second leading cause of death globally and is responsible for an estimated 9.6 million deaths in 2018, in which the most common cancers include, lung cancer (about 2.09 million cases), breast cancer (about 2.09 million cases), colorectal cancer (about 1.80 million cases), prostate cancer (about 1.28 million cases), skin cancer (about 1.04 million cases), and gastric cancer (about 1.03 million cases).

Treatments for cancers may vary with the type of cancer and how advanced it is. Conventional treatments for cancers include surgery, radiation therapy, and chemotherapy. Such treatments usually cause a variety of complications or side effects, such as infection, blood clot, bleeding, nausea and vomiting, diarrhea, nerve or muscle damage, incontinence, and sex and fertility issues. Immunotherapy provides an alternative strategy for cancer treatment that aims at specifically stimulating immune responses of a subject against cancer cells via, for example, blocking immune checkpoints, or enhancing the ability of immune cells (e.g., T cells or B cells) to target and destroy cancer cells. Serious adverse effects associated with immunotherapy-medicated overstimulation or non-specific toxicity have been reported in cancer patients, including neurotoxicity, cytokine release syndrome (CRS), allergy, organ inflammation, and autoimmune disorders.

It is therefore of great importance to develop efficient cancer treatment specifically targeting cancer cells without affecting normal cells and/or tissues.

### SUMMARY OF THE INVENTION

The present disclosure is based, at least in part, on the development of rapid and efficient processes for producing immune cells (e.g., T cells) armed with bi-specific antibodies comprising at least one binding moiety to a tumor associated antigens. The processes disclosed herein allow for production of armed immune cells, which may be enriched with one or more particular types of T cells (e.g., CD4+, CD8+, Treg, etc.), in a short period (e.g., less than 6 hours) without the need of in vitro cell culture, expansion, and/or activation. The armed immune cells thus produced may be used for therapeutic purposes, for example, in cancer treatment, with no further processing (e.g., cell culture, expansion, and/or activation). As such, the preparation process disclosed herein can be performed at a point of care to produce the armed immune cell therapeutics and using such to treat a patient.

Accordingly, in some aspects, the present disclosure features a method for preparing armed immune cells, comprising: (i) isolating a population of CD3⁺ immune cells from a human blood sample; and (ii) contacting the population of immune cells with a bi-specific antibody specific to CD3 and a tumor associated antigen (TAA) to produce armed immune cells, which display the bi-specific antibody on the cell surface. In some instances, step (i) and step (ii) can be performed concurrently. In some instances, the CD3⁺ immune cells are not cultured in vitro prior to step (ii).

In some embodiments, the population of CD3+ immune cells comprise CD8+ T cells, CD4+ T cells, natural killer (NK) T cells, regulatory T (T_{reg}) cells, or a combination thereof. In some examples, the population of CD3+ immune cells are substantially CD8+ T cells, e.g., containing at least 80% of CD8+ T cells. In other examples, the population of CD3+ immune cells are substantially CD4+ T cells, e.g., containing at least 80% of CD4+ T cells. In yet other examples, the population of CD3+ immune cells are substantially NK T cells, e.g., containing at least 80% of NK T cells. In still other examples, the population of CD3+ immune cells are substantially T_{reg} cells, e.g., containing at least 80% of Treg cells.

In some embodiments, the human blood sample is a peripheral blood mononuclear cell (PBMC) sample. For example, the human blood sample may be obtained from a human donor. In specific examples, the human blood sample may be obtained from a human cancer patient.

In any of the methods disclosed herein, step (i) may comprise negative selection. Alternatively, step (i) comprises positive selection. Alternatively or in addition, step (ii) may comprise incubating the population of immune cells with the bi-specific antibody at a temperature of about 4-37° C. for about 30 minutes to 2 hours.

In some embodiments, the method disclosed herein may further comprise administering the armed immune cells to a human patient in need thereof. In some instances, the armed immune cells are autologous to the human patient. In other embodiments, the method disclosed herein may further comprise placing the armed immune cells in a cryopreservation solution for storage.

In any of the methods disclosed herein, the bi-specific antibody may comprise a first antigen binding fragment that binds human CD3, which comprises a first heavy chain that comprises a first heavy chain variable region (V_{H}) and a first light chain that comprises a first light chain variable region (V_{L}). The first V_{H} may comprise the same heavy chain complementary determining regions (CDRs) or no more than 5 amino acid variations relative to a first reference antibody and the first V_{L} comprises the same light chain CDRs or no more than 5 amino acid variations relative to the reference antibody, which may be CTA.02, CTA.03, CTA.04, or CTA.05. In some examples, the first heavy chain and the first light chain comprise the same V_{H} and V_{L} as the reference antibody.

In some embodiments, the bi-specific antibody comprises a second antigen binding fragment that binds the TAA. Exemplary TAAs include, but are not limited to, CD20, CD19, EGFR, HER2, PSMA, CEA, EpCAM, FAP, PD-L1, CD38, CD33, cMET, CD47, TRAIL-R2, mesothelin, or GD2.

In other aspects, the present disclosure provides a method for treating cancer, comprising administering to a human cancer patient a population of armed immune cells, which is obtained from any of the preparation methods disclosed herein. In some instances, the human cancer patient comprises cancer cells expressing the TAA, to which the bi-specific antibody binds. In some examples, the population of armed immune cells are autologous to the human patient. In other instances, the population of armed immune cells are allogenic to the human patient.

In some embodiments, the human cancer patient may have melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, or myeloma.

Also provided herein are armed immune cells such as armed T cells prepared by any of the methods disclosed here for use in cancer treatment and uses of such armed immune cells for manufacturing a medicament for cancer treatment.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic diagram depicting an exemplary process for producing armed T cells.

FIG. 2 is a diagram showing in vitro cytotoxicity of armed T cells co-cultured with cancer cells.

FIG. 3A-3D include diagrams showing cytokine production by armed T cells when co-cultured with cancer cells. FIG. 3A: IL-2. FIG. 3B: TNF-alpha. FIG. 3C: Perforin.

FIG. 3D: Granzyme B.

FIGS. 4A-4K include diagrams showing cytotoxicity of T cells armed with exemplary bispecific antibodies prepared by the methods disclosed herein against target cancer cells.

FIG. 4A: cytotoxicity of T cells armed with CTA.03Fab/CTAT01scFv against Raji cells (CD19+/CD20+). FIG. 4B: cytotoxicity of T cells armed with CTA.03Fab/CTAT.02scFv against Raji cells (CD19+/CD20+). FIG. 4C: cytotoxicity of T cells armed with CTA.03Fab/CTAT.03scFv against A549 cells (EGFR+). FIG. 4D: cytotoxicity of T cells armed with CTA.03Fab/CTAT.04scFv against MCF-7 cells (HER2+). FIG. 4E: cytotoxicity of T cells armed with CTA.03Fab/CTAT.05scFv against LNCaP cells (PSMA+/EpCAM+). FIG. 4F: cytotoxicity of T cells armed with CTA.03Fab/CTAT.07scFv against LNCaP cells (PSMA+/EpCAM+). FIG. 4G: cytotoxicity of T cells armed with CTA.03Fab/CTAT.08scFv against FAP overexpressed 3T3 cells. FIG. 4H: cytotoxicity of T cells armed with CTA.03Fab/CTAT.09scFv against MDA-MB-231 cells (PDL1). FIG. 4I: cytotoxicity of T cells armed with CTA.03Fab/CTAT.010scFv against Raji cells (CD38+). FIG. 4J: cytotoxicity of T cells armed with CTA.03Fab/CTAT.12scFv against A549 cells (cMET+). FIG. 4K: cytotoxicity of T cells armed with CTA.03Fab/CTAT.13scFv against MCF-7 cells (CD47+).

FIGS. 5A and 5B include diagrams showing cytotoxicity of T cells armed with bispecific antibodies comprising anti-CD3 moiety in combination with different binding moieties to a tumor antigen. FIG. 5A: cytotoxicity of T cells armed with bispecific antibodies comprising the same anti-CD3 moiety and different anti-EGFR moieties against A549 cells. FIG. 5B: cytotoxicity of T cells armed with bispecific antibodies comprising the same anti-CD3 moiety and different anti-CD20 moieties against Raji cells.

FIGS. 6A and 6B include diagrams showing cytotoxicity of T cells armed with bispecific antibodies comprising different anti-CD3 moieties and the same anti-tumor antigen moiety. FIG. 6A: cytotoxicity of T cells armed with bispecific antibodies comprising different anti-CD3 moieties in Fab format as indicated and the same anti-CD19 moiety against Raji cells. FIG. 6B: cytotoxicity of T cells armed with bispecific antibodies comprising different anti-CD3 moieties in scFv format as indicated and the same anti-CD19 moiety against Raji cells.

FIGS. 7A and 7B include diagrams showing cytotoxicity of T cells armed with bispecific antibodies comprising mutated anti-CD3 or anti-CD19 moieties. FIG. 7A: various pairs of anti-CD3 mutants and anti-CD19 mutants as indicated. FIG. 7B: various anti-CD3 mutants in combination with an anti-PSMA moiety against LNCaP (a prostate cancer cell line).

### DETAILED DESCRIPTION OF THE INVENTION

Disclosed herein are rapid and efficient processes for producing immune cells (e.g., T cells) armed with bi-specific antibodies (BsAb) for use in treating a target disease such as cancer. The BsAbs used herein are capable of attaching to CD3+ immune cells via the anti-CD3 binding moiety in the BsAb. The BsAb disclosed herein further comprise a binding moiety specific to a tumor associated antigen (TAA) such as those disclosed herein. As used herein, the term “an armed immune cell” or “an armed T cell” refers to an immune cell (e.g., a T cell) that displays a bispecific antibody as disclosed herein via binding of the anti-CD3 moiety in the bispecific antibody to a cell surface CD3 molecule. Via the anti-TAA moiety in the bispecific antibody on the cell surface, an armed immune cell is capable of targeting disease cells (e.g., cancer cells) that express the TAA, thereby eliciting immune responses against the disease cells.

The preparation processes disclosed herein allow for production of armed immune cells, in a short period (e.g., less than 6 hours) without the need of in vitro cell culture, expansion, and/or activation. The armed immune cells may be enriched with one or more particular types of T cells (e.g., CD4+, CD8+, Treg, etc.). The armed immune cells thus produced may be used for therapeutic purposes, for example, in cancer treatment, with no further processing (e.g., cell culture, expansion, and/or activation).

As such, the preparation process disclosed herein can substantially reduce costs for manufacturing armed immune cells as compared with conventional approaches. Convention approaches involve lengthy in vitro cell culture processes, which would require large amounts of cell culture materials and instruments. The preparation process disclosed herein does not need lengthy in vitro culturing, thereby reducing both costs and time for producing effective armed immune cells. Further, the preparation process provided herein can be performed at a point of care (e.g., a hospital, a clinic, or in an operation room) to produce the armed immune cell therapeutics, which can be used to treat the patient on site. Moreover, the preparation process provided herein can be performed by collecting immune cells from a patient, converting such to armed immune cells as disclosed herein, and delivering the armed immune cells back to the patient in a short time period (e.g., less than 6 hours). Accordingly, prior to the immune cell transplantation, the patient does not need to treated by a lymphodepletion chemotherapy, which is a common procedure of conventional immune cell therapy.

### I. Rapid Process for Producing Armed Immune Cells

In some aspects, the present disclosure provides a rapid method for producing armed immune cells such as armed T cells without the need of in vitro cell culture. Such a method may comprise: (i) isolating a population of CD3+ immune cells from a human blood sample; and (ii) contacting the population of immune cells with a bi-specific antibody specific to CD3 and a tumor associated antigen (TAA) to produce armed immune cells, which display the bi-specific antibody on the cell surface.

### A. Sources of Immune Cells

The immune cells such as T cells for use in preparing the armed cells may be obtained from a suitable source, for example, one or more mammal donors. In some examples, the immune cells may be obtained from one or more human donors, such as healthy human donors or human patients. In some examples, the immune cells comprise parent primary T obtained from one or more human donors (e.g., 2, 3, 4, or 5 human donors). Alternatively, the immune cells may be differentiated from precursor T cells obtained from one or more suitable donor or stem cells such as hematopoietic stem cells or inducible pluripotent stem cells (iPSC), which may be cultured in vitro.

In some embodiments, the immune cells may be derived from one or more suitable mammals, for example, one or more human donors. In some examples, the immune cells may be obtained from a human cancer patient, who may be the recipient of the armed immune cells thus prepared. The immune cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, the immune cells such as T cells can be obtained from a unit of blood collected from a subject using any number of techniques known to the skilled person, such as sedimentation, e.g., FICOLL™ separation.

In some instances, the immune cells may comprise primary T cells isolated from one or more human donors. Such T cells are terminally differentiated, not transformed, depend on cytokines and/or growth factors for growth, and/or have stable genomes. Alternatively, the T cells may be derived from stem cells (e.g., HSCs or iPSCs) via in vitro differentiation.

### B. Immune Cell Enrichment

One or more specific types of immune cells may be isolated from the immune cells obtained from a suitable source as disclosed herein to produce an isolated immune cell population for use in making the armed immune cells. In some examples, suitable T cells can be isolated from a mixture of immune cells (e.g., those described herein) to produce an isolated T cell population, which may be enriched with one or more specific types of T cells. For example, after isolation of peripheral blood mononuclear cells (PBMC), both cytotoxic and helper T lymphocytes can be sorted into naive, memory, and effector T cell subpopulations. In some instances, the isolated T cell population may be enriched with CD4+ T cells. In other instances, the isolated T cell population may be enriched with CD8+ T cells. In yet other instances, the isolated T cell population may be enriched with CD4+ T cells and CD8+ T cells. Alternatively, the isolated T cell population may be enriched with NK T cells. In other examples, the isolated T cell population may be enriched with regulatory T (Treg) cells. In some examples, the isolated T cell population may comprise a mixture of T cell subtypes, for example CD4+ T cells and CD8+ T cells.

The isolated T cell population may be prepared by conventional methods or following guidance provided herein. For example, a specific T cell subtype may be isolated by negative selection to exclude undesired cells. Alternatively, a specific T cell subtype may be isolated by positive selection. In some examples, a commercially available positive or negative selection kit (e.g., those disclosed in Example 1 below) may be used.

In some examples, CD4⁺ T cells and/or CD8⁺ T cells can be isolated from a suitable blood cell source, such as those described herein, using any method known in the art or those disclosed herein, for example, using antibodies capable of binding to specific cell-surface biomarkers for the target T cells, e.g., antibodies specific to CD4 and/or antibodies specific to CD8. For example, enriching CD4⁺ T cells and CD8⁺ T cells can be performed using anti-CD4 and/or anti-CD8 antibodies conjugated to magnetic beads. A cell population comprising CD4⁺ and/or CD8⁺ T cells can be incubated with such magnetic beads under suitable conditions for a suitable period allowing for binding of the target T cells to the magnetic beads via the antibodies conjugated to the beads. Non-bound cells can be washed and CD4⁺ and CD8⁺ T cells bound to the beads can be collected using routine methods.

The enriched T cells (e.g., CD4⁺ T cells, CD8⁺ T cells, NK T cells, Treg cells, or a combination thereof) may be evaluated for features such as cell viability and/or purity of the target T cells following routine practice. In some embodiments, the T cell population from the enrichment step disclosed here may contain substantially CD8+ T cells, for example, at least 80% (e.g., at least 85%, at least 90%, at least 95%, at least 98% or higher) of the T cells are CD8+ T cells. In some embodiments, the T cell population from the enrichment step disclosed here may contain substantially CD4+ T cells, for example, at least 80% (e.g., at least 85%, at least 90%, at least 95%, at least 98% or higher) of the T cells are CD4+ T cells. In some embodiments, the T cell population from the enrichment step disclosed here may contain substantially Treg cells, for example, at least 80% (e.g., at least 85%, at least 90%, at least 95%, at least 98% or higher) of the T cells are Treg cells. In some embodiments, the T cell population from the enrichment step disclosed here may contain substantially NK T cells, for example, at least 80% (e.g., at least 85%, at least 90%, at least 95%, at least 98% or higher) of the T cells are NK T cells.

### C. Preparation of Armed Immune Cells

The isolated immune cell populations as disclosed herein (e.g., CD4⁺ T cells, CD8⁺ T cells, NK T cells, Treg cells, or a combination thereof) can then be incubated with a BsAb as also disclosed herein under a suitable temperature for a suitable period to allow for attachment of the BsAb onto the surface of the immune cells, thereby producing the armed immune cells. It is not necessary to culture the immune cells in vitro for expansion and/or activation prior to incubation with the BsAb, thereby shortening the whole preparation process. This would also allow for on-site preparation of the armed immune cells for therapeutic uses at a point of care.

In some embodiments, the isolated immune cells may be incubated with the BsAb at a temperature ranging from about 4° C. to about 37° C. In some examples, the incubation step can be performed at 4° C. In other examples, the incubation step can be performed at room temperature (e.g., at 20° C. or 25° C.). In yet other examples. In some examples, the incubation step can be performed at 37° C.

Alternative or in addition, the isolated immune cells may be incubated with the BsAb at a suitable temperature as disclosed herein for about 30 minutes to about 2 hours. In some examples, the isolated immune cells may be incubated with the BsAb for about 30 minutes. In some examples, the isolated immune cells may be incubated with the BsAb for about 45 minutes. In some examples, the isolated immune cells may be incubated with the BsAb for about 60 minutes. In some examples, the isolated immune cells may be incubated with the BsAb for about 90 minutes. In some examples, the isolated immune cells may be incubated with the BsAb for about 2 hours.

The armed immune cells thus formed may be used directly for any of the therapeutic purposes disclosed herein, for example, in a cancer treatment. Alternatively, the armed immune cells may be suspended in a cryopreservation solution for storage, which can be used for therapeutic purposes at a later time.

### II. Bi-Specific Antibodies for Producing Armed Immune Cells

The bispecific antibodies (BsAb) disclosed herein are capable of binding to CD3 (e.g., CD3+ cells) and a tumor associated antigen (TAA) (e.g., cancer cells expressing the TAA on cell surface). An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. A bispecific antibody as disclosed herein comprises two antigen-binding moieties, one of which binds CD3 such as human CD3 and the other one of which binds a tumor associated antigen such as those disclosed herein.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an antibody moiety disclosed herein may share the same heavy chain and/or light chain complementary determining regions (CDRs) or the same V_{H} and/or V_{L} chains as a reference antibody. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-CD19 antibodies may have the same V_{H}, the same V_{L}, or both as compared to an exemplary antibody described herein.

In some embodiments, an antibody moiety disclosed herein may share a certain level of sequence identity as compared with a reference sequence. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, an antibody moiety disclosed herein may have one or more amino acid variations relative to a reference antibody. The amino acid residue variations as disclosed in the present disclosure (e.g., in framework regions and/or in CDRs) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

### A. Bispecific Antibodies

The bispecific antibodies disclosed herein comprise a CD3 binding moiety (anti-CD3 moiety) and a TAA binding moiety (anti-TAA moiety).

### (i) CD3 Binding Moieties

The anti-CD3 moiety in any of the bispecific antibodies disclosed herein comprises an antigen-binding fragment specific to a CD3 molecule, for example, human CD3. In some embodiments, the anti-CD3 moiety comprises a heavy chain variable region (V_{H}) and a light chain variable region (V_{L}). In some instances, the anti-CD3 moiety may be derived from a reference anti-CD3 antibody. Exemplary reference anti-CD3 antibodies include CTA.02, CTA.03, CTA.04, or CTA.05. The structural information of these reference anti-CD3 antibodies are provided in Table 1 below (heavy chain and light chain complementary determining regions (CDRs) based on the Kabat scheme are in boldface and underlined).

TABLE 1
Reference Anti-CD3 Antibodies

| | | | SEQ ID |
|---|---|---|---|
| Reference Antibody | | Amino Acid Sequences | NO |
| CTA.02 | VH CDR1 | RYTMH | 1 |
| | VH CDR2 | YINPSRGYTNYNQKFKD | 2 |
| | VH CDR3 | YYDDHYCLDY | 3 |
| | VH | QVQLQQSGAELARPGASVKMSCKASGYTFTRYTMHWVKQRP | 4 |
| | | GQGLEWIGYINPSRGYTNYNQKFKDKATLTTDKSSSTAYMQ | |
| | | LSSLTSEDSAVYYCARYYDDHYCLDYWGQGTTLTVSS | |
| | VL CDR1 | SASSSVSYMN | 5 |
| | VL CDR2 | DTSKLAS | 6 |
| | VL CDR3 | QQWSSNPFT | 7 |
| | VL | QIVLTQSPAIMSASPGEKVTMTCSASSSVSYMNWYQQKSGT | 8 |
| | | SPKRWIYDTSKLASGVPAHFRGSGSGTSYSLTISGMEAEDA | |
| | | ATYYCQQWSSNPFTFGSGTKLEINR | |
| | scFv | GGGSGGGQVQLQQSGAELARPGASVKMSCKASGYTFTRYTM | 9 |
| | | HWVKQRPGQGLEWIGYINPSRGYTNYNQKFKDKATLTTDKS | 250 (no N- |
| | | SSTAYMQLSSLTSEDSAVYYCARYYDDHYCLDYWGQGTTLT | terminus |
| | | VSSGGGGSGGGGSGGGGSQIVLTQSPAIMSASPGEKVTMTC | peptide |
| | | SASSSVSYMNWYQQKSGTSPKRWIYDTSKLASGVPAHFRGS | linker) |
| | | GSGTSYSLTISGMEAEDAATYYCQQWSSNPFTFGSGTKLEI | |
| | | NR | |
| | VH-CH1 | QVQLQQSGAELARPGASVKMSCKASGYTFTRYTMHWVKQRP | 10 |
| | | GQGLEWIGYINPSRGYTNYNQKFKDKATLTTDKSSSTAYMQ | |
| | | LSSLTSEDSAVYYCARYYDDHYCLDYWGQGTTLTVSSAKTT | |
| | | APSVYPLAPVCGGTTGSSVTLGCLVKGYFPEPVTLTWNSGS | |
| | | LSSGVHTFPAVLQSDLYTLSSSVTVISSTWPSQSITCNVAH | |
| | | PASSTKVDKKI | |
| | VL-Ck | QIVLTQSPAIMSASPGEKVTMTCSASSSVSYMNWYQQKSGT | 11 |
| | | SPKRWIYDTSKLASGVPAHFRGSGSGTSYSLTISGMEAEDA | |
| | | ATYYCQQWSSNPFTFGSGTKLEINRADTAPTVSIFPPSSEQ | |
| | | LTSGGASVVCFLNNFYPKDINVKWKIDGSERQNGVLNSWTD | |
| | | QDSKDSTYSMSSTLTLTKDEYERHNSYTCEATHKISTSPIV | |
| | | KSFNRNECS | |
| CTA.03 | VH CDR1 | SFPMA | 12 |
| | VH CDR2 | TISTSGGRTYYRDSVKG | 13 |
| | VH CDR3 | FRQYSGGFDY | 14 |
| | VH | EVQLLESGGGLVQPGGSLRLSCAASGFTFSSFPMAWVRQAP | 15 |
| | | GKGLEWVSTISTSGGRTYYRDSVKGRFTISRDNSKNTLYLQ | |
| | | MNSLRAEDTAVYYCAKFRQYSGGFDYWGQGTLVTVSS | |
| | VL CDR1 | TLSSGNIENNYVH | 16 |
| | VL CDR2 | DDDKRPD | 17 |
| | VL | DDDKRPE | 18 |
| | CDR2-02 | | |
| | VL CDR3 | HSYVSSFNV | 19 |
| | VL | DIQLTQPNSVSTSLGSTVKLSCTLSSGNIENNYVHWYQLYE | 20 |
| | | GRSPTTMIYDDDKRPDGVPDRFSGSIDRSSNSAFLTIHNVA | |
| | | IEDEAIYFCHSYVSSFNVFGGGTKLTVLRQ | |
| | VL-01 | DIQLTQPNSVSTSLGSTVKLSCTLSSGNIENNYVHWYQLYE | 21 |
| | | GRSPTTMIYDDDKRPDAVPDRFSGSIDRSSNSAFLTIHNVA | |
| | | IEDEAIYFCHSYVSSFNVFGGGTKLTVLRQ | |
| | VL-02 | DIQLTQPNSVSTSLGSTVKLSCTLSSGNIENNYVHWYQLYE | 249 |
| | | GRSPTTMIYDDDKRPEGVPDRFSGSIDRSSNSAFLTIHNVA | |
| | | IEDEAIYFCHSYVSSFNVFGGGTKLTVLRQ | |
| | scFv | GGGSGGGEVQLLESGGGLVQPGGSLRLSCAASGFTFSSFPM | 22 |
| | | AWVRQAPGKGLEWVSTISTSGGRTYYRDSVKGRFTISRDNS | 251 (no N- |
| | | KNTLYLQMNSLRAEDTAVYYCAKFRQYSGGFDYWGQGTLVT | terminus |
| | | VSSGGGGSGGGGSGGGGSDIQLTQPNSVSTSLGSTVKLSCT | peptide |
| | | LSSGNIENNYVHWYQLYEGRSPTTMIYDDDKRPDGVPDRFS | linker) |
| | | GSIDRSSNSAFLTIHNVAIEDEAIYFCHSYVSSFNVFGGGT | |
| | | KLTVLRQ | |
| | VH-CH1 | EVQLLESGGGLVQPGGSLRLSCAASGFTFSSFPMAWVRQAP | 23 |
| | | GKGLEWVSTISTSGGRTYYRDSVKGRFTISRDNSKNTLYLQ | |
| | | MNSLRAEDTAVYYCAKFRQYSGGFDYWGQGTLVTVSSASTK | |
| | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSC | |
| | VL-CA | DIQLTQPNSVSTSLGSTVKLSCTLSSGNIENNYVHWYQLYE | 24 |
| | | GRSPTTMIYDDDKRPDGVPDRFSGSIDRSSNSAFLTIHNVA | |
| | | IEDEAIYFCHSYVSSFNVFGGGTKLTVLRQPKAAPSVTLEP | |
| | | PSSEELQANKATLVCLISDFYPGAVTVAWKADSSPVKAGVE | |
| | | TTTPSKQSNNKYAASSYLSLTPEQWKSHRSYSCQVTHEGST | |
| | | VEKTVAPTECS | |
| | VL-Ca-01 | DIQLTQPNSVSTSLGSTVKLSCTLSSGNIENNYVHWYQLYE | 228 |
| | | GRSPTTMIYDDDKRPDAVPDRFSGSIDRSSNSAFLTIHNVA | |
| | | IEDEAIYFCHSYVSSFNVFGGGTKLTVLRQPKAAPSVTLEP | |
| | | PSSEELQANKATLVCLISDFYPGAVTVAWKADSSPVKAGVE | |
| | | TTTPSKQSNNKYAASSYLSLTPEQWKSHRSYSCQVTHEGST | |
| | | VEKTVAPTECS | |
| | VL-CA-02 | DIQLTQPNSVSTSLGSTVKLSCTLSSGNIENNYVHWYQLYE | 25 |
| | | GRSPTTMIYDDDKRPEGVPDRFSGSIDRSSNSAFLTIHNVA | |
| | | IEDEAIYFCHSYVSSFNVFGGGTKLTVLRQPKAAPSVTLFP | |
| | | PSSEELQANKATLVCLISDFYPGAVTVAWKADSSPVKAGVE | |
| | | TTTPSKQSNNKYAASSYLSLTPEQWKSHRSYSCQVTHEGST | |
| | | VEKTVAPTECS | |
| CTA.04 | VH CDR1 | RYTMH | 26 |
| | VH CDR2 | YINPSRGYTNYNQKVKD | 27 |
| | VH CDR3 | YYDDHYCLDY | 28 |
| | VH | QVQLVQSGGGVVQPGRSLRLSCKASGYTFTRYTMHWVRQAP | 29 |
| | | GKGLEWIGYINPSRGYTNYNQKVKDRFTISRDNSKNTAFLQ | |
| | | MDSLRPEDTGVYFCARYYDDHYCLDYWGQGTPVTVSS | |
| | VL CDR1 | SASSSVSYMN | 30 |
| | VL CDR2 | DTSKLAS | 31 |
| | VL CDR3 | QQWSSNPFT | 32 |
| | VL | DIQMTQSPSSLSASVGDRVTITCSASSSVSYMNWYQQTPGK | 33 |
| | | APKRWIYDTSKLASGVPSRFSGSGSGTDYTFTISSLQPEDI | |
| | | ATYYCQQWSSNPFTFGQGTKLQITR | |
| | scFv | GGGSGGGQVQLVQSGGGVVQPGRSLRLSCKASGYTFTRYTM | 34 |
| | | HWVRQAPGKGLEWIGYINPSRGYTNYNQKVKDRFTISRDNS | 252 (no N- |
| | | KNTAFLQMDSLRPEDTGVYFCARYYDDHYCLDYWGQGTPVT | terminus |
| | | VSSGGGGSGGGGSGGGGSDIQMTQSPSSLSASVGDRVTITC | peptide |
| | | SASSSVSYMNWYQQTPGKAPKRWIYDTSKLASGVPSRFSGS | linker) |
| | | GSGTDYTFTISSLQPEDIATYYCQQWSSNPFTFGQGTKLQI | |
| | | TR | |
| | VH-CH1 | QVQLVQSGGGVVQPGRSLRLSCKASGYTFTRYTMHWVRQAP | 35 |
| | | GKGLEWIGYINPSRGYTNYNQKVKDRFTISRDNSKNTAFLQ | |
| | | MDSLRPEDTGVYFCARYYDDHYCLDYWGQGTPVTVSSASTK | |
| | | GPSVFPLAPSSKSTSGGTAALGCLVKDYFPEPVTVSWNSGA | |
| | | LTSGVHTFPAVLQSSGLYSLSSVVTVPSSSLGTQTYICNVN | |
| | | HKPSNTKVDKKVEPKSC | |
| | VL-Ck | DIQMTQSPSSLSASVGDRVTITCSASSSVSYMNWYQQTPGK | 36 |
| | | APKRWIYDTSKLASGVPSRFSGSGSGTDYTFTISSLQPEDI | |
| | | ATYYCQQWSSNPFTFGQGTKLQITRTVAAPSVFIFPPSDEQ | |
| | | LKSGTASVVCLLNNFYPREAKVQWKVDNALQSGNSQESVTE | |
| | | QDSKDSTYSLSSTLTLSKADYEKHKVYACEVTHQGLSSPVT | |
| | | KSFNRGEC | |
| CTA.05 | VH CDR1 | SYTMH | 37 |
| | VH CDR2 | YINPRSGYTHYNQKLKD | 38 |
| | VH CDR3 | SAYYDYDGFAY | 39 |
| | VH | QVQLVQSGAEVKKPGASVKVSCKASGYTFISYTMHWVRQAP | 40 |
| | | GQGLEWMGYINPRSGYTHYNQKLKDKATLTADKSASTAYME | |
| | | LSSLRSEDTAVYYCARSAYYDYDGFAYWGQGTLVTVSS | |
| | VL CDR1 | SASSSVSYMN | 41 |
| | VL CDR2 | DISKLAS | 42 |
| | VL CDR3 | QQWSSNPPT | 43 |
| | VL | DIQMTQSPSSLSASVGDRVTITCSASSSVSYMNWYQQKPGK | 44 |
| | | APKRLIYDTSKLASGVPSRFSGSGSGTDFTLTISSLQPEDF | |
| | | ATYYCQQWSSNPPTFGGGTKVEIK | |
| | scFv | GGGSGGGQVQLVQSGAEVKKPGASVKVSCKASGYTFISYTM | 45 |
| | | HWVRQAPGQGLEWMGYINPRSGYTHYNQKLKDKATLTADKS | 253 (no N- |
| | | ASTAYMELSSLRSEDTAVYYCARSAYYDYDGFAYWGQGTLV | terminus |
| | | TVSSGGGGSGGGGSGGGGSDIQMTQSPSSLSASVGDRVTIT | peptide |
| | | CSASSSVSYMNWYQQKPGKAPKRLIYDTSKLASGVPSRFSG | linker) |
| | | SGSGTDFTLTISSLQPEDFATYYCQQWSSNPPTFGGGTKVE | |
| | | IK | |
| | VH-CH1 | QVQLVQSGAEVKKPGASVKVSCKASGYTFISYTMHWVRQAP | 46 |
| | | GQGLEWMGYINPRSGYTHYNQKLKDKATLTADKSASTAYME | |
| | | LSSLRSEDTAVYYCARSAYYDYDGFAYWGQGTLVTVSSAST | |
| | | KGPSVFPLAPCSRSTSESTAALGCLVKDYFPEPVTVSWNSG | |
| | | ALTSGVHTFPAVLQSSGLYSLSSVAIVTVPSSNFGTQTYTC | |
| | | NVDHKPSNTKVDKTV | |
| | VL-Ck | DIQMTQSPSSLSASVGDRVTITCSASSSVSYMNWYQQKPGK | 47 |
| | | APKRLIYDTSKLASGVPSRFSGSGSGTDFTLTISSLQPEDF | |
| | | ATYYCQQWSSNPPTFGGGTKVEIKRTVAAPSVFIFPPSDEQ | |
| | | LKSGTASVVCLLNNFYPREAKVQWKVDNALQSGNSQESVTE | |
| | | QDSKDSTYSLSSTLTLSKADYEKHKLYACEVTHQGLSSPVT | |
| | | KSFNRGEC | |

An anti-CD3 binding moiety (and an anti-TAA binding moiety disclosed below) derived from a reference antibody refers to binding moieties having substantially similar structural and functional features as the reference antibody. Structurally, the binding moiety may have the same heavy and/or light chain complementary determining regions or the same V_{H} and/or V_{L} chains as the reference antibody. Alternatively, the binding moiety may only have a limited number of amino acid variations in one or more of the framework regions and/or in one or more of the CDRs without significantly affecting its binding affinity and binding specificity relative to the reference antibody.

In some embodiments, the anti-CD3 binding moiety may comprise the same heavy chain CDRs as those in antibody CTA.02, which are provided in Table 1 above. Alternatively or in addition, the anti-CD3 binding moiety may have the same light chain CDRs as those in antibody CTA.02, which are also provided in Table 1 above. Such an anti-CD3 binding moiety may comprise the same V_{H} and/or V_{L} chains as CTA.02. Alternatively, the anti-CD3 binding moiety may comprise amino acid variations in one or more of the framework regions relative to the corresponding framework regions in CTA.02. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.02.

In some embodiments, the anti-CD3 moiety may comprise a certain level of variations in one or more of the CDRs relative to those of CTA.02. For example, the anti-CD3 moiety may comprise heavy chain CDRs that are at least 80% (e.g., 85%, 90%, 95%, or 98%) sequence identity, individually or collectively, as compared with the V_{H} CDRs of CTA.02. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.02. As used herein, “individually” means that one CDR of an antibody shares the indicated sequence identity relative to the corresponding CDR of a reference antibody (e.g., the anti-CD3 reference antibodies provided in Table 1 above or any of the anti-TAA reference antibodies disclosed below). “Collectively” means that three V_{H} or V_{L} CDRs of an antibody in combination share the indicated sequence identity relative the corresponding three V_{H} or V_{L} CDRs of the reference antibody in combination.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.02. In some instances, the anti-CD3 moiety may comprise the same heavy chain CDR3 as the heavy chain CDR3 of CTA.02 and comprise one or more amino acid variations in one or more of the other heavy chain and light chain CDRs.

In some embodiments, the anti-CD3 binding moiety may comprise the same heavy chain CDRs as those in antibody CTA.03, which are provided in Table 1 above. Alternatively or in addition, the anti-CD3 binding moiety may have the same light chain CDRs as those in antibody CTA.03, which are also provided in Table 1 above. Such an anti-CD3 binding moiety may comprise the same V_{H} and/or V_{L} chains as CTA.03. Alternatively, the anti-CD3 binding moiety may comprise amino acid variations in one or more of the framework regions relative to the corresponding framework regions in CTA.03. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.03. In one specific example, the anti-CD3 moiety disclosed herein comprises a mutation at position G58 of the V_{L} chain relative to CTA.03, for example, an amino acid residue substitution (e.g., G58A). See, e.g., CTA.03 V_{L}-01 in Table 1 above.

In some embodiments, the anti-CD3 moiety may comprise a certain level of variations in one or more of the CDRs relative to those of CTA.03. For example, the anti-CD3 moiety may comprise heavy chain CDRs that are at least 80% (e.g., 85%, 90%, 95%, or 98%) sequence identity, individually or collectively, as compared with the V_{H} CDRs of CTA.03. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.03.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.03. In some instances, the anti-CD3 moiety may comprise the same heavy chain CDR3 as the heavy chain CDR3 of CTA.03 and comprise one or more amino acid variations in one or more of the other heavy chain and light chain CDRs. In specific examples, the anti-CD3 moiety disclosed herein may comprise a mutation at position D57 of the V_{L} chain relative to that of CTA.03, for example, an amino acid residue substitution such as D57E. See, e.g., CTA.03 V_{L}-02 in Table 1.

In some examples, the anti-CD3 binding moiety may comprise the same heavy chain CDRs as those in antibody CTA.04, which are provided in Table 1 above. Alternatively or in addition, the anti-CD3 binding moiety may have the same light chain CDRs as those in antibody CTA.04, which are also provided in Table 1 above. Such an anti-CD3 binding moiety may comprise the same V_{H} and/or V_{L} chains as CTA.04. Alternatively, the anti-CD3 binding moiety may comprise amino acid variations in one or more of the framework regions relative to the corresponding framework regions in CTA.04. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.04.

In some embodiments, the anti-CD3 moiety may comprise a certain level of variations in one or more of the CDRs relative to those of CTA.04. For example, the anti-CD3 moiety may comprise heavy chain CDRs that are at least 80% (e.g., 85%, 90%, 95%, or 98%) sequence identity, individually or collectively, as compared with the V_{H} CDRs of CTA.04. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.04.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.04. In some instances, the anti-CD3 moiety may comprise the same heavy chain CDR3 as the heavy chain CDR3 of CTA.04 and comprise one or more amino acid variations in one or more of the other heavy chain and light chain CDRs.

In some examples, the anti-CD3 binding moiety may comprise the same heavy chain CDRs as those in antibody CTA.05, which are provided in Table 1 above. Alternatively or in addition, the anti-CD3 binding moiety may have the same light chain CDRs as those in antibody CTA.05, which are also provided in Table 1 above. Such an anti-CD3 binding moiety may comprise the same V_{H} and/or V_{L} chains as CTA.05. Alternatively, the anti-CD3 binding moiety may comprise amino acid variations in one or more of the framework regions relative to the corresponding framework regions in CTA.05. For example, the anti-CD3 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTA.05.

In some embodiments, the anti-CD3 moiety may comprise a certain level of variations in one or more of the CDRs relative to those of CTA.05. For example, the anti-CD3 moiety may comprise heavy chain CDRs that are at least 80% (e.g., 85%, 90%, 95%, or 98%) sequence identity, individually or collectively, as compared with the V_{H} CDRs of CTA.05. Alternatively or in addition, the anti-CD3 antibody may comprise light chain CDRs that are at least 80% (e.g., 85%, 90%, 95%, or 98%) sequence identity, individually or collectively, as compared with the V_{L} CDRs as CTA.05.

In some instances, the anti-CD3 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTA.05. In some instances, the anti-CD3 moiety may comprise the same heavy chain CDR3 as the heavy chain CDR3 of CTA.05 and comprise one or more amino acid variations in one or more of the other heavy chain and light chain CDRs.

### (ii) TAA Binding Moiety

In addition to the anti-CD3 binding moiety, any of the bispecific antibodies disclosed herein further comprises a second binding moiety specific to a tumor associated antigen. The term “tumor-associated antigen” (TAA) is well-understood in the art, and refers to a molecule that is differentially expressed on and/in cancerous cells relative to non-cancerous cells of the same cell type. Non-limiting examples of TAA include CD5, CD19, CD20, CD22, CD23, CD25, CD27, CD30, CD33, CD34, CD37, CD38, CD40, CD43, CD44v6, CD47, CD50, CD52, CD56, CD63, CD72a, CD74, CD78, CD79a, CD79b, CD86, CD134, CD137, CD138, CD248, CD319, αvβ3, α5β1, human epidermal growth factor receptor (EGFR or HER1), HER2, HER3, HER4, vascular endothelial growth factor receptor 1 (VEGFR-1), VEGFR-2, VEGFR-3, TRAIL-R2, carbohydrate antigen 19-9 (CA 19-9), carbohydrate antigen 125 (CA 125), carcinoembryonic antigen (CEA), mucin 1 (MUC 1), MUC2, MUC3, MUC4, MUC5, MUC7, ganglioside GD2, ganglioside GD3, ganglioside GM2, carbonic anhydrase IX (CAIX), sonic hedgehog (SHH), melanoma chondroitin sulfate proteoglycan (MCSP), chondroitin sulfate proteoglycan 4 (CSPG4), six-transmembrane epithelial antigen of prostate (STEAP), A33 antigen, desmoglein-2 (Dsg2), Dsg3, Dsg4, E-cadherin neoepitope, fetal nicotinic acetylcholine receptor (fnAChR), muellerian inhibitory substance receptor type II (MISIIR), tumor-associated antigen L6 (TAL6), Thomsen-Friedenreich (TF) antigen, EPHA1, EPHA2, EPHA3, EPHA4, EPHA7, EPHA8, EPHA10, EPHB4, cancer testis antigen (CTA), NY-BR1, tumor-associated glycoprotein 72 (TAG-72), alpha-fetoprotein (AFP), brother of the regulator of the imprinted site (BORIS), B-cell activating factor (BAFF), extradomain-B fibronectin (EDB-FN), glycoprotein A33 (GPA33), tenascin-C (TNC), melanoma-associated antigen (MAGE), GAGE, BAGE, prostate stem cell antigen (PSCA), mesothelin, mucine-related Tn, Sialyl Tn, globo H, stage-specific embryonic antigen-4 (SSEA-4), epithelial cell adhesion molecule (EpCAM), cytotoxic T-lymphocyte-associated protein 4 (CTLA-4), programmed cell death 1 (PD-1), programmed cell death 1 ligand 1 (PD-L1), prostate-specific membrane antigen (PSMA), fibroblast activation protein (FAP), vascular cell adhesion protein 1 (VCAM-1), insulin-like growth factor receptor (IGFR), or hepatocyte growth factor receptor (HGFR).

In some embodiments, the anti-TAA binding moiety comprises a heavy chain variable region (V_{H}) and a light chain variable region (V_{L}). In some examples, the anti-TAA binding moiety is specific to CD20 (e.g., human CD20). In some examples, the anti-TAA binding moiety is specific to CD19 (e.g., human CD19). In some examples, the anti-TAA binding moiety is specific to EGFR (e.g., human EGFR). In some examples, the anti-TAA binding moiety is specific to HER2 (e.g., human HER2). In some examples, the anti-TAA binding moiety is specific to PSMA (e.g., human PSMA). In some examples, the anti-TAA binding moiety is specific to CEA (e.g., human CEA). In some examples, the anti-TAA binding moiety is specific to EpCAM (e.g., human EpCAM). In some examples, the anti-TAA binding moiety is specific to FAP (e.g., human FAP). In some examples, the anti-TAA binding moiety is specific to PDL1 (e.g., human PDL1). In some examples, the anti-TAA binding moiety is specific to CD38 (e.g., human CD38). In some examples, the anti-TAA binding moiety is specific to CD33 (e.g., human CD33). In some examples, the anti-TAA binding moiety is specific to HGFR (cMET) (e.g., human cMET). In some examples, the anti-TAA binding moiety is specific to CD47 (e.g., human CD47). In some examples, the anti-TAA binding moiety is specific to TRAIL-R2 (e.g., human TRAIL-R2). In some examples, the anti-TAA binding moiety is specific to mesothelin (e.g., human mesothelin). In some examples, the anti-TAA binding moiety is specific to GD2 (e.g., human GD2).

In some instances, the anti-TAA moiety may be derived from a reference anti-TAA antibody. Exemplary reference anti-TAA antibodies include CTAT.01-CTAT.16. The structural information of these reference anti-CD3 antibodies are provided in Table 2 below (heavy chain and light chain complementary determining regions (CDRs) based on the Kabat scheme are in boldface and underlined).

TABLE 2
Reference Anti-Tumor Associated Antigen Antibodies

| Reference Antibody | | | SEQ |
|---|---|---|---|
| (TAA) | | Amino Acid Sequences | ID NO |
| CTAT.01 | VH | SYNMH | 48 |
| (CD20) | CDR1 | | |
| | VH | AIYPGNGDTSYNQKFKG | 49 |
| | CDR2 | | |
| | VH | STYYGGDWYFNV | 50 |
| | CDR3 | | |
| | VH | QVQLQQPGAELVKPGASVKMSCKASGYTFTSYNMHWVKQTPGRGLEW | 51 |
| | | IGAIYPGNGDTSYNQKFKGKATLTADKSSSTAYMQLSSLTSEDSAVY | |
| | | YCARSTYYGGDWYFNVWGAGTTVTVSA | |
| | VL | RASSSVSYIH | 52 |
| | CDR1 | | |
| | VL | ATSNLAS | 53 |
| | CDR2 | | |
| | VL | QQWTSNPPT | 54 |
| | CDR3 | | |
| | VL | QIVLSQSPAILSASPGEKVTMTCRASSSVSYIHWFQQKPGSSPKPWI | 55 |
| | | YATSNLASGVPVRFSGSGSGTSYSLTISRVEAEDAATYYCQQWTSNP | |
| | | PTFGGGTKLEIK | |
| | scFv | GGGSGGGQVQLQQPGAELVKPGASVKMSCKASGYTFTSYNMHWVKQT | 56 |
| | | PGRGLEWIGAIYPGNGDTSYNQKFKGKATLTADKSSSTAYMQLSSLT | 254 (no N- |
| | | SEDSAVYYCARSTYYGGDWYFNVWGAGTTVTVSAGGGGSGGGGSGGG | terminus |
| | | GSQIVLSQSPAILSASPGEKVTMTCRASSSVSYIHWFQQKPGSSPKP | peptide |
| | | WIYATSNLASGVPVRFSGSGSGTSYSLTISRVEAEDAATYYCQQWTS | linker) |
| | | NPPTFGGGTKLEIK | |
| | VH-CH1 | QVQLQQPGAELVKPGASVKMSCKASGYTFTSYNMHWVKQTPGRGLEW | 57 |
| | | IGAIYPGNGDTSYNQKFKGKATLTADKSSSTAYMQLSSLTSEDSAVY | |
| | | YCARSTYYGGDWYFNVWGAGTTVTVSAASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKKAEPKSC | |
| | VL-Ck | QIVLSQSPAILSASPGEKVTMTCRASSSVSYIHWFQQKPGSSPKPWI | 58 |
| | | YATSNLASGVPVRFSGSGSGTSYSLTISRVEAEDAATYYCQQWTSNP | |
| | | PTFGGGTKLEIKRTVAAPSVFIFPPSDEQLKSGTASVVCLLNNFYPR | |
| | | EAKVQWKVDNALQSGNSQESVTEQDSKDSTYSLSSTLILSKADYEKH | |
| | | KVYACEVTHQGLSSPVTKSFNRGEC | |
| CTAT.02 | VH | DYGVS | 59 |
| (CD19) | CDR1 | | |
| | VH | VIWGSETTYYNSALKS | 60 |
| | CDR2 | | |
| | VH | HYYYGGSYAMDY | 61 |
| | CDR3 | | |
| | VH | EVKLQESGPGLVAPSQSLSVTCTVSGVSLPDYGVSWIRQPPRKGLEW | 62 |
| | | LGVIWGSETTYYNSALKSRLTIIKDNSKSQVFLKMNSLQTDDTAIYY | |
| | | CAKHYYYGGSYAMDYWGQGTSVTVSS | |
| | VL | RASQDISKYLN | 63 |
| | CDR1 | | |
| | VL | HTSRLHS | 64 |
| | CDR2 | | |
| | VL | QQGNTLPYT | 65 |
| | CDR3 | | |
| | VL | DIQMTQTTSSLSASLGDRVTISCRASQDISKYLNWYQQKPDGTVKLL | 66 |
| | | IYHTSRLHSGVPSRFSGSGSGTDYSLTISNLEQEDIATYFCQQGNTL | |
| | | PYTFGGGTKLEIT | |
| | VL-01 | DIQMTQTTSSLSASLGDRVTISCRASQDISKYLNWYQQKPDATVKLL | 67 |
| | | IYHTSRLHSGVPSRFSGSGSGTDYSLTISNLEQEDIATYFCQQGNTL | |
| | | PYTFGGGTKLEIT | |
| | VL-02 | DIQMTQTTSSLSASLGDRVTISCRASQDISKYLNWYQQKPEGTVKLL | 68 |
| | | IYHTSRLHSGVPSRFSGSGSGTDYSLTISNLEQEDIATYFCQQGNTL | |
| | | PYTFGGGTKLEIT | |
| | scFv | GGGSGGGEVKLQESGPGLVAPSQSLSVTCTVSGVSLPDYGVSWIRQP | 69 |
| | | PRKGLEWLGVIWGSETTYYNSALKSRLTIIKDNSKSQVFLKMNSLQT | 255 (no N- |
| | | DDTAIYYCAKHYYYGGSYAMDYWGQGTSVTVSSGGGGSGGGGSGGGG | terminus |
| | | SDIQMTQTTSSLSASLGDRVTISCRASQDISKYLNWYQQKPDGTVKL | peptide |
| | | LIYHTSRLHSGVPSRFSGSGSGTDYSLTISNLEQEDIATYFCQQGNT | linker) |
| | | LPYTFGGGTKLEIT | |
| | scFv-01 | GGGSGGGEVKLQESGPGLVAPSQSLSVTCTVSGVSLPDYGVSWIRQP | 70 |
| | | PRKGLEWLGVIWGSETTYYNSALKSRLTIIKDNSKSQVELKMNSLQT | 256 (no N- |
| | | DDTAIYYCAKHYYYGGSYAMDYWGQGTSVTVSSGGGGSGGGGSGGGG | terminus |
| | | SDIQMTQTTSSLSASLGDRVTISCRASQDISKYLNWYQQKPDATVKL | peptide |
| | | LIYHTSRLHSGVPSRFSGSGSGTDYSLTISNLEQEDIATYFCQQGNT | linker) |
| | | LPYTFGGGTKLEIT | |
| | scFv-02 | GGGSGGGEVKLQESGPGLVAPSQSLSVTCTVSGVSLPDYGVSWIRQP | 71 |
| | | PRKGLEWLGVIWGSETTYYNSALKSRLTIIKDNSKSQVFLKMNSLQT | 257 (no N- |
| | | DDTAIYYCAKHYYYGGSYAMDYWGQGTSVTVSSGGGGSGGGGSGGGG | terminus |
| | | SDIQMTQTTSSLSASLGDRVTISCRASQDISKYLNWYQQKPEGTVKL | peptide |
| | | LIYHTSRLHSGVPSRFSGSGSGTDYSLTISNLEQEDIATYFCQQGNT | linker) |
| | | LPYTFGGGTKLEIT | |
| | VH-CH1 | EVKLQESGPGLVAPSQSLSVTCTVSGVSLPDYGVSWIRQPPRKGLEW | 72 |
| | | LGVIWGSETTYYNSALKSRLTIIKDNSKSQVFLKMNSLQTDDTAIYY | |
| | | CAKHYYYGGSYAMDYWGQGTSVTVSSASTKGPSVFPLAPSSKSTSGG | |
| | | TAALGCLVKDYFPEPVTVSWNSGALTSGVHTFPAVLQSSGLYSLSSV | |
| | | VTVPSSSLGTQTYICNVNHKPSNTKVDKRVEPKSCDK | |
| | VL-Ck | DIQMTQTTSSLSASLGDRVTISCRASQDISKYLNWYQQKPDGTVKLL | 73 |
| | | IYHTSRLHSGVPSRFSGSGSGTDYSLTISNLEQEDIATYFCQQGNTL | |
| | | PYTFGGGTKLEITRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSFNRGEC | |
| CTAT.03 | VH | NYGVH | 74 |
| (EGFR) | CDR1 | | |
| | VH | VIWSGGNTDYNTPFTS | 75 |
| | CDR2 | | |
| | VH | ALTYYDYEFAY | 76 |
| | CDR3 | | |
| | VH | QVQLKQSGPGLVQPSQSLSITCTVSGFSLTNYGVHWVRQSPGKGLEW | 77 |
| | | LGVIWSGGNTDYNTPFTSRLSINKDNSKSQVFFKMNSLQSNDTAIYY | |
| | | CARALTYYDYEFAYWGQGTLVTVSA | |
| | VL | RASQSIGTNIH | 78 |
| | CDR1 | | |
| | VL | YASESIS | 79 |
| | CDR2 | | |
| | VL | QQNNNWPTT | 80 |
| | CDR3 | | |
| | VL | DILLTQSPVILSVSPGERVSFSCRASQSIGTNIHWYQQRTNGSPRLL | 81 |
| | | IKYASESISGIPSRFSGSGSGTDFTLSINSVESEDIADYYCQQNNNW | |
| | | PTTFGAGTKLELK | |
| | scFv | GGGSGGGQVQLKQSGPGLVQPSQSLSITCTVSGFSLTNYGVHWVRQS | 82 |
| | | PGKGLEWLGVIWSGGNTDYNTPFTSRLSINKDNSKSQVFFKMNSLQS | 258 (no N- |
| | | NDTAIYYCARALTYYDYEFAYWGQGTLVTVSAGGGGSGGGGSGGGGS | terminus |
| | | DILLTQSPVILSVSPGERVSFSCRASQSIGTNIHWYQQRTNGSPRLL | peptide |
| | | IKYASESISGIPSRFSGSGSGTDFTLSINSVESEDIADYYCQQNNNW | linker) |
| | | PTTFGAGTKLELK | |
| | VH-CH1 | QVQLKQSGPGLVQPSQSLSITCTVSGFSLTNYGVHWVRQSPGKGLEW | 83 |
| | | LGVIWSGGNTDYNTPFTSRLSINKDNSKSQVFFKMNSLQSNDTAIYY | |
| | | CARALTYYDYEFAYWGQGTLVTVSAASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKRVEPKSCDK | |
| | VL-Ck | DILLTQSPVILSVSPGERVSFSCRASQSIGTNIHWYQQRINGSPRLL | 84 |
| | | IKYASESISGIPSRFSGSGSGTDFTLSINSVESEDIADYYCQQNNNW | |
| | | PTTFGAGTKLELKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSFNRGEC | |
| CTAT.04 | VH | DTYIH | 85 |
| (HER2) | CDR1 | | |
| | VH | RIYPTNGYTRYADSVKG | 86 |
| | CDR2 | | |
| | VH | WGGDGFYAMDY | 7 |
| | CDR3 | | |
| | VH | EVQLVESGGGLVQPGGSLRLSCAASGFNIKDTYIHWVRQAPGKGLEW | 88 |
| | | VARIYPTNGYTRYADSVKGRFTISADTSKNTAYLQMNSLRAEDTAVY | |
| | | YCSRWGGDGFYAMDYWGQGTLVTVSS | |
| | VL | RASQDVNTAVA | 89 |
| | CDR1 | | |
| | VL | SASFLYS | 90 |
| | CDR2 | | |
| | VL | QQHYTTPPT | 91 |
| | CDR3 | | |
| | VL | DIQMTQSPSSLSASVGDRVTITCRASQDVNTAVAWYQQKPGKAPKLL | 92 |
| | | IYSASFLYSGVPSRFSGSRSGTDFTLTISSLQPEDFATYYCQQHYTT | |
| | | PPTFGQGTKVEIK | |
| | scFv | GGGSGGGEVQLVESGGGLVQPGGSLRLSCAASGFNIKDTYIHWVRQA | 93 |
| | | PGKGLEWVARIYPTNGYTRYADSVKGRFTISADTSKNTAYLQMNSLR | 259 (no N- |
| | | AEDTAVYYCSRWGGDGFYAMDYWGQGTLVTVSSGGGGSGGGGSGGGG | terminus |
| | | SDIQMTQSPSSLSASVGDRVTITCRASQDVNTAVAWYQQKPGKAPKL | peptide |
| | | LIYSASFLYSGVPSRFSGSRSGTDFTLTISSLQPEDFATYYCQQHYT | linker) |
| | | TPPTFGQGTKVEIK | |
| | VH-CH1 | EVQLVESGGGLVQPGGSLRLSCAASGFNIKDTYIHWVRQAPGKGLEW | 94 |
| | | VARIYPTNGYTRYADSVKGRFTISADTSKNTAYLQMNSLRAEDTAVY | |
| | | YCSRWGGDGFYAMDYWGQGTLVTVSSASTKGPSVFPLAPSSKSTSGG | |
| | | TAALGCLVKDYFPEPVTVSWNSGALTSGVHTFPAVLQSSGLYSLSSV | |
| | | VTVPSSSLGTQTYICNVNHKPSNTKVDKRVEPKSCDK | |
| | VL-Ck | DIQMTQSPSSLSASVGDRVTITCRASQDVNTAVAWYQQKPGKAPKLL | 95 |
| | | IYSASFLYSGVPSRFSGSRSGTDFTLTISSLQPEDFATYYCQQHYTT | |
| | | PPTFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSENRGEC | |
| CTAT.05 | VH | EYTIH | 96 |
| (PSMA) | CDR1 | | |
| | VH | NINPNNGGTTYNQKFED | 97 |
| | CDR2 | | |
| | VH | GWNFDY | 98 |
| | CDR3 | | |
| | VH | EVQLVQSGAEVKKPGASVKISCKTSGYTFTEYTIHWVKQASGKGLEW | 99 |
| | | IGNINPNNGGTTYNQKFEDRATLTVDKSTSTAYMELSSLRSEDTAVY | |
| | | YCAAGWNFDYWGQGTTVTVSS | |
| | VL | KASQDVGTAVD | 100 |
| | CDR1 | | |
| | VL | WASTRHT | 101 |
| | CDR2 | | |
| | VL | QQYNSYPLT | 102 |
| | CDR3 | | |
| | VL | DIVMTQSPSSLSASVGDRVTITCKASQDVGTAVDWYQQKPGKAPKLL | 103 |
| | | IYWASTRHTGVPDRFTGSGSGTDFTLTISSLQPEDFADYFCQQYNSY | |
| | | PLTFGGGTKLEIK | |
| | scFv | GGGSGGGEVQLVQSGAEVKKPGASVKISCKTSGYTFTEYTIHWVKQA | 104 |
| | | SGKGLEWIGNINPNNGGTTYNQKFEDRATLTVDKSTSTAYMELSSLR | 260 (no N- |
| | | SEDTAVYYCAAGWNFDYWGQGTTVTVSSGGGGSGGGGSGGGGSDIVM | terminus |
| | | TQSPSSLSASVGDRVTITCKASQDVGTAVDWYQQKPGKAPKLLIYWA | peptide |
| | | STRHTGVPDRFTGSGSGTDFTLTISSLQPEDFADYFCQQYNSYPLTF | linker) |
| | | GGGTKLEIK | |
| | VH-CH1 | EVQLVQSGAEVKKPGASVKISCKTSGYTFTEYTIHWVKQASGKGLEW | 105 |
| | | IGNINPNNGGTTYNQKFEDRATLTVDKSTSTAYMELSSLRSEDTAVY | |
| | | YCAAGWNFDYWGQGTTVTVSSASTKGPSVFPLAPSSKSTSGGTAALG | |
| | | CLVKDYFPEPVTVSWNSGALTSGVHTFPAVLQSSGLYSLSSVVTVPS | |
| | | SSLGTQTYICNVNHKPSNTKVDKRVEPKSCDK | |
| | VL-Ck | DIVMTQSPSSLSASVGDRVTITCKASQDVGTAVDWYQQKPGKAPKLL | 106 |
| | | IYWASTRHTGVPDRFTGSGSGTDFTLTISSLQPEDFADYFCQQYNSY | |
| | | PLTFGGGTKLEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKLYACEVTHQGLSSPVTKSFNRGEC | |
| CTAT.06 | VH | EYGMN | 107 |
| (CEA) | CDR1 | | |
| | VH | WINTKSGEATYVEEFKG | 108 |
| | CDR2 | | |
| | VH | WDFYDYVDEAMY | 109 |
| | CDR3 | | |
| | VH | QVQLVQSGSELKKPGASVKVSCKASGYTFTEYGMNVWRQAPGQGLEW | 110 |
| | | MGWINTKSGEATYVEEFKGRFVFSLDTSVSTAYLQISSLKAEDTAVY | |
| | | YCARWDFYDYVDEAMYWGQGTTVTVSS | |
| | VL | KASQTVSANVA | 111 |
| | CDR1 | | |
| | VL | LASYRYR | 112 |
| | CDR2 | | |
| | VL | HQYYTYPLFT | 113 |
| | CDR3 | | |
| | VL | DIQMTQSPSSLSASVGDRVTITCKASQTVSANVAWYQQKPGKAPKLL | 114 |
| | | IYLASYRYRGVPSRFSGSGSGTDFTLTISSLQPEDFATYYCHQYYTY | |
| | | PLFTFGQGTKLEIK | |
| | scFv | GGGSGGGQVQLVQSGSELKKPGASVKVSCKASGYTFTEYGMNVWRQA | 115 |
| | | PGQGLEWMGWINTKSGEATYVEEFKGRFVFSLDTSVSTAYLQISSLK | 261 (no N- |
| | | AEDTAVYYCARWDFYDYVDEAMYWGQGTTVTVSSGGGGSGGGGSGGG | terminus |
| | | GSDIQMTQSPSSLSASVGDRVTITCKASQTVSANVAWYQQKPGKAPK | peptide |
| | | LLIYLASYRYRGVPSRFSGSGSGTDFTLTISSLQPEDFATYYCHQYY | linker) |
| | | TYPLFTFGQGTKLEIK | |
| | VH-CH1 | QVQLVQSGSELKKPGASVKVSCKASGYTFTEYGMNVWRQAPGQGLEW | 116 |
| | | MGWINTKSGEATYVEEFKGRFVFSLDTSVSTAYLQISSLKAEDTAVY | |
| | | YCARWDFYDYVDEAMYWGQGTTVTVSSASTKGPSVFPLAPSSKSTSG | |
| | | GTAALGCLVKDYFPEPVTVSWNSGALTSGVHTFPAVLQSSGLYSLSS | |
| | | VVTVPSSSLGTQTYICNVNHKPSNTKVDKRVEPKSCDK | |
| | VL-Ck | DIQMTQSPSSLSASVGDRVTITCKASQTVSANVAWYQQKPGKAPKLL | 117 |
| | | IYLASYRYRGVPSRFSGSGSGTDFTLTISSLQPEDFATYYCHQYYTY | |
| | | PLFTFGQGTKLEIKRTVAAPSVFIFPPSDEQLKSGTASVVCLLNNFY | |
| | | PREAKVQWKVDNALQSGNSQESVTEQDSKDSTYSLSSTLTLSKADYE | |
| | | KHKLYACEVTHQGLSSPVTKSFNRGEC | |
| CTAT.07 | VH | NYGMN | 118 |
| (EpCAM) | CDR1 | | |
| | VH | WINTYTGESTYADSFKG | 119 |
| | CDR2 | | |
| | VH | FAIKGDY | 120 |
| | CDR3 | | |
| | VH | EVQLVQSGPGLVQPGGSVRISCAASGYTFTNYGMNWVKQAPGKGLEW | 121 |
| | | MGWINTYTGESTYADSFKGRFTFSLDTSASAAYLQINSLRAEDTAVY | |
| | | YCARFAIKGDYWGQGTLLTVSS | |
| | VL | RSTKSLLHSNGITYLY | 122 |
| | CDR1 | | |
| | VL | QMSNLAS | 123 |
| | CDR2 | | |
| | VL | AQNLEIPRT | 124 |
| | CDR3 | | |
| | VL | DIQMTQSPSSLSASVGDRVTITCRSTKSLLHSNGITYLYWYQQKPGK | 125 |
| | | APKLLIYQMSNLASGVPSRFSSSGSGTDFTLTISSLQPEDFATYYCA | |
| | | QNLEIPRTFGQGTKVELK | |
| | scFv | GGGSGGGEVQLVQSGPGLVQPGGSVRISCAASGYTFTNYGMNWVKQA | 126 |
| | | PGKGLEWMGWINTYTGESTYADSFKGRFTFSLDTSASAAYLQINSLR | 262 (no N- |
| | | AEDTAVYYCARFAIKGDYWGQGTLLTVSSGGGGSGGGGSGGGGSDIQ | terminus |
| | | MTQSPSSLSASVGDRVTITCRSTKSLLHSNGITYLYWYQQKPGKAPK | peptide |
| | | LLIYQMSNLASGVPSRFSSSGSGTDFTLTISSLQPEDFATYYCAQNL | linker) |
| | | EIPRTFGQGTKVELK | |
| | VH-CH1 | EVQLVQSGPGLVQPGGSVRISCAASGYTFTNYGMNWVKQAPGKGLEW | 127 |
| | | MGWINTYTGESTYADSFKGRFTFSLDTSASAAYLQINSLRAEDTAVY | |
| | | YCARFAIKGDYWGQGTLLTVSSASTKGPSVFPLAPSSKSTSGGTAAL | |
| | | GCLVKDYFPEPVTVSWNSGALTSGVHTFPAVLQSSGLYSLSSVVTVP | |
| | | SSSLGTQTYICNVNHKPSNTKVDKRVEPKSCDK | |
| | VL-Ck | DIQMTQSPSSLSASVGDRVTITCRSTKSLLHSNGITYLYWYQQKPGK | 128 |
| | | APKLLIYQMSNLASGVPSRFSSSGSGTDFTLTISSLQPEDFATYYCA | |
| | | QNLEIPRTFGQGTKVELKRTVAAPSVFIFPPSDEQLKSGTASVVCLL | |
| | | NNFYPREAKVQWKVDNALQSGNSQESVTEQDSKDSTYSLSSTLTLSK | |
| | | ADYEKHKLYACEVTHQGLSSPVTKSFNRGEC | |
| CTAT.08 | VH | EYTIH | 129 |
| (FAP) | CDR1 | | |
| | VH | GINPNNGIPNYNQKFKG | 130 |
| | CDR2 | | |
| | VH | RRIAYGYDEGHAMDY | 131 |
| | CDR3 | | |
| | VH | QVQLVQSGAEVKKPGASVKVSCKTSRYTFTEYTIHWVRQAPGQRLEW | 132 |
| | | IGGINPNNGIPNYNQKFKGRVTITVDTSASTAYMELSSLRSEDTAVY | |
| | | YCARRRIAYGYDEGHAMDYWGQGTLVTVSS | |
| | VL | KSSQSLLYSRNQKNYLA | 133 |
| | CDR1 | | |
| | VL | WASTRES | 134 |
| | CDR2 | | |
| | VL | QQYFSYPLT | 135 |
| | CDR3 | | |
| | VL | DIVMTQSPDSLAVSLGERATINCKSSQSLLYSRNQKNYLAWYQQKPG | 136 |
| | | QPPKLLIFWASTRESGVPDRFSGSGFGTDFTLTISSLQAEDVAVYYC | |
| | | QQYFSYPLTFGQGTKVEIK | |
| | scFv | GGGSGGGQVQLVQSGAEVKKPGASVKVSCKTSRYTFTEYTIHWVRQA | 137 |
| | | PGQRLEWIGGINPNNGIPNYNQKFKGRVTITVDTSASTAYMELSSLR | 263 (no N- |
| | | SEDTAVYYCARRRIAYGYDEGHAMDYWGQGTLVTVSSGGGGSGGGGS | terminus |
| | | GGGGSDIVMTQSPDSLAVSLGERATINCKSSQSLLYSRNQKNYLAWY | peptide |
| | | QQKPGQPPKLLIFWASTRESGVPDRFSGSGFGTDFTLTISSLQAEDV | linker) |
| | | AVYYCQQYFSYPLTFGQGTKVEIK | |
| | VH-CH1 | QVQLVQSGAEVKKPGASVKVSCKTSRYTFTEYTIHWVRQAPGQRLEW | 138 |
| | | IGGINPNNGIPNYNQKFKGRVTITVDTSASTAYMELSSLRSEDTAVY | |
| | | YCARRRIAYGYDEGHAMDYWGQGTLVTVSSASTKGPSVFPLAPSSKS | |
| | | TSGGTAALGCLVKDYFPEPVTVSWNSGALTSGVHTFPAVLQSSGLYS | |
| | | LSSVVTVPSSSLGTQTYICNVNHKPSNTKVDKRVEPKSCDK | |
| | VL-Ck | DIVMTQSPDSLAVSLGERATINCKSSQSLLYSRNQKNYLAWYQQKPG | 139 |
| | | QPPKLLIFWASTRESGVPDRFSGSGFGTDFTLTISSLQAEDVAVYYC | |
| | | QQYFSYPLTFGQGTKVEIKRTVAAPSVFIFPPSDEQLKSGTASVVCL | |
| | | LNNFYPREAKVQWKVDNALQSGNSQESVTEQDSKDSTYSLSSTLTLS | |
| | | KADYEKHKLYACEVTHQGLSSPVTKSFNRGEC | |
| CTAT.09 | VH | DSWIH | 140 |
| (PDL1) | CDR1 | | |
| | VH | WISPYGGSTYYADSVKG | 141 |
| | CDR2 | | |
| | VH | RHWPGGFDY | 142 |
| | CDR3 | | |
| | VH | EVQLVESGGGLVQPGGSLRLSCAASGFTFSDSWIHWVRQAPGKGLEW | 143 |
| | | VAWISPYGGSTYYADSVKGRFTISADTSKNTAYLQMNSLRAEDTAVY | |
| | | YCARRHWPGGFDYWGQGTLVTVSS | |
| | VL | RASQDVSTAVA | 144 |
| | CDR1 | | |
| | VL | SASFLYS | 145 |
| | CDR2 | | |
| | VL | QQYLYHPAT | 146 |
| | CDR3 | | |
| | VL | DIQMTQSPSSLSASVGDRVTITCRASQDVSTAVAWYQQKPGKAPKLL | 147 |
| | | IYSASFLYSGVPSRFSGSGSGTDFTLTISSLQPEDFATYYCQQYLYH | |
| | | PATFGQGTKVEIK | |
| | scFv | GGGSGGGEVQLVESGGGLVQPGGSLRLSCAASGFTFSDSWIHWVRQA | 148 |
| | | PGKGLEWVAWISPYGGSTYYADSVKGRFTISADTSKNTAYLQMNSLR | 264 (no N- |
| | | AEDTAVYYCARRHWPGGFDYWGQGTLVTVSSGGGGSGGGGSGGGGSD | terminus |
| | | IQMTQSPSSLSASVGDRVTITCRASQDVSTAVAWYQQKPGKAPKLLI | peptide |
| | | YSASFLYSGVPSRFSGSGSGTDFTLTISSLQPEDFATYYCQQYLYHP | linker) |
| | | ATFGQGTKVEIK | |
| | VH-CH1 | EVQLVESGGGLVQPGGSLRLSCAASGFTFSDSWIHWVRQAPGKGLEW | 149 |
| | | VAWISPYGGSTYYADSVKGRFTISADTSKNTAYLQMNSLRAEDTAVY | |
| | | YCARRHWPGGFDYWGQGTLVTVSSASTKGPSVFPLAPSSKSTSGGTA | |
| | | ALGCLVKDYFPEPVTVSWNSGALTSGVHTFPAVLQSSGLYSLSSVVT | |
| | | VPSSSLGTQTYICNVNHKPSNTKVDKKVEPKSCDK | |
| | VL-Ck | DIQMTQSPSSLSASVGDRVTITCRASQDVSTAVAWYQQKPGKAPKLL | 150 |
| | | IYSASFLYSGVPSRFSGSGSGTDFTLTISSLQPEDFATYYCQQYLYH | |
| | | PATFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CTAT.10 | VH | SFAMS | 151 |
| (CD38) | CDR1 | | |
| | VH | AISGSGGGTYYADSVKG | 152 |
| | CDR2 | | |
| | VH | DKILWFGEPVFDY | 153 |
| | CDR3 | | |
| | VH | EVQLLESGGGLVQPGGSLRLSCAVSGFTFNSFAMSWVRQAPGKGLEW | 154 |
| | | VSAISGSGGGTYYADSVKGRFTISRDNSKNTLYLQMNSLRAEDTAVY | |
| | | FCAKDKILWFGEPVFDYWGQGTLVTVSS | |
| | VL | RASQSVSSYLA | 155 |
| | CDR1 | | |
| | VL | DASNRAT | 156 |
| | CDR2 | | |
| | VL | QQRSNWPPT | 157 |
| | CDR3 | | |
| | VL | EIVLTQSPATLSLSPGERATLSCRASQSVSSYLAWYQQKPGQAPRLL | 158 |
| | | IYDASNRATGIPARFSGSGSGTDFTLTISSLEPEDFAVYYCQQRSNW | |
| | | PPTFGQGTKVEIK | |
| | scFv | GGGSGGGEVQLLESGGGLVQPGGSLRLSCAVSGFTENSFAMSWVRQA | 159 |
| | | PGKGLEWVSAISGSGGGTYYADSVKGRFTISRDNSKNTLYLQMNSLR | 265 (no N- |
| | | AEDTAVYFCAKDKILWFGEPVEDYWGQGTLVTVSSGGGGSGGGGSGG | terminus |
| | | GGSEIVLTQSPATLSLSPGERATLSCRASQSVSSYLAWYQQKPGQAP | peptide |
| | | RLLIYDASNRATGIPARFSGSGSGTDFTLTISSLEPEDFAVYYCQQR | linker) |
| | | SNWPPTFGQGTKVEIK | |
| | VH-CH1 | EVQLLESGGGLVQPGGSLRLSCAVSGFTFNSFAMSWVRQAPGKGLEW | 160 |
| | | VSAISGSGGGTYYADSVKGRFTISRDNSKNTLYLQMNSLRAEDTAVY | |
| | | FCAKDKILWFGEPVFDYWGQGTLVTVSSASTKGPSVEPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDK | |
| | VL-Ck | EIVLTQSPATLSLSPGERATLSCRASQSVSSYLAWYQQKPGQAPRLL | 161 |
| | | IYDASNRATGIPARFSGSGSGTDFTLTISSLEPEDFAVYYCQQRSNW | |
| | | PPTFGQGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSENRGEC | |
| CTAT.11 | VH | DSNIH | 162 |
| (CD33) | CDR1 | | |
| | VH | YIYPYNGGTDYNQKFKN | 163 |
| | CDR2 | | |
| | VH | GNPWLAY | 164 |
| | CDR3 | | |
| | VH | EVQLVQSGAEVKKPGSSVKVSCKASGYTITDSNIHWVRQAPGQSLEW | 165 |
| | | IGYIYPYNGGTDYNQKFKNRATLTVDNPTNTAYMELSSLRSEDTAFY | |
| | | YCVNGNPWLAYWGQGTLVTVSS | |
| | VL | RASESLDNYGIRFLT | 166 |
| | CDR1 | | |
| | VL | AASNQGS | 167 |
| | CDR2 | | |
| | VL | QQTKEVPWS | 168 |
| | CDR3 | | |
| | VL | DIQLTQSPSTLSASVGDRVTITCRASESLDNYGIRFLTWFQQKPGKA | 169 |
| | | PKLLMYAASNQGSGVPSRFSGSGSGTEFTLTISSLQPDDFATYYCQQ | |
| | | TKEVPWSFGQGTKVEVK | |
| | scFv | GGGSGGGEVQLVQSGAEVKKPGSSVKVSCKASGYTITDSNIHWVRQA | 170 |
| | | PGQSLEWIGYIYPYNGGTDYNQKFKNRATLTVDNPTNTAYMELSSLR | 266 (no N- |
| | | SEDTAFYYCVNGNPWLAYWGQGTLVTVSSGGGGSGGGGSGGGGSDIQ | terminus |
| | | LTQSPSTLSASVGDRVTITCRASESLDNYGIRFLTWFQQKPGKAPKL | peptide |
| | | LMYAASNQGSGVPSRFSGSGSGTEFTLTISSLQPDDFATYYCQQTKE | linker) |
| | | VPWSFGQGTKVEVK | |
| | VH-CH1 | EVQLVQSGAEVKKPGSSVKVSCKASGYTITDSNIHWVRQAPGQSLEW | 171 |
| | | IGYIYPYNGGTDYNQKFKNRATLTVDNPTNTAYMELSSLRSEDTAFY | |
| | | YCVNGNPWLAYWGQGTLVTVSSASTKGPSVFPLAPCSRSTSESTAAL | |
| | | GCLVKDYFPEPVTVSWNSGALTSGVHTFPAVLQSSGLYSLSSVVTVP | |
| | | SSSLGTKTYTCNVDHKPSNTKVDKRV | |
| | VL-Ck | DIQLTQSPSTLSASVGDRVTITCRASESLDNYGIRFLTWFQQKPGKA | 172 |
| | | PKLLMYAASNQGSGVPSRFSGSGSGTEFTLTISSLQPDDFATYYCQQ | |
| | | TKEVPWSFGQGTKVEVKRTVAAPSVFIFPPSDEQLKSGTASVVCLLN | |
| | | NFYPREAKVQWKVDNALQSGNSQESVTEQDSKDSTYSLSSTLTLSKA | |
| | | DYEKHKVYACEVTHQGLSSPVTKSFNRGEC | |
| CTAT.12 | VH | SYWLH | 173 |
| (HGFR | CDR1 | | |
| (cMET)) | VH | MIDPSNSDTRFNPNFKD | 174 |
| | CDR2 | | |
| | VH | YRSYVTPLDY | 175 |
| | CDR3 | | |
| | VH | EVQLVESGGGLVQPGGSLRLSCAASGYTFTSYWLHWVRQAPGKGLEW | 176 |
| | | VGMIDPSNSDTRFNPNFKDRFTISADTSKNTAYLQMNSLRAEDTAVY | |
| | | YCATYRSYVTPLDYWGQGTLVTVSS | |
| | VL | KSSQSLLYTSSQKNYLA | 177 |
| | CDR1 | | |
| | VL | WASTRES | 178 |
| | CDR2 | | |
| | VL | QQYYAYPWT | 179 |
| | CDR3 | | |
| | VL | DIQMTQSPSSLSASVGDRVTITCKSSQSLLYTSSQKNYLAWYQQKPG | 180 |
| | | KAPKLLIYWASTRESGVPSRFSGSGSGTDFTLTISSLQPEDFATYYC | |
| | | QQYYAYPWTFGQGTKVEIK | |
| | scFv | GGGSGGGEVQLVESGGGLVQPGGSLRLSCAASGYTFTSYWLHWVRQA | 181 |
| | | PGKGLEWVGMIDPSNSDTRENPNFKDRFTISADTSKNTAYLQMNSLR | 267 (no N- |
| | | AEDTAVYYCATYRSYVTPLDYWGQGTLVTVSSGGGGSGGGGSGGGGS | terminus |
| | | DIQMTQSPSSLSASVGDRVTITCKSSQSLLYTSSQKNYLAWYQQKPG | peptide |
| | | KAPKLLIYWASTRESGVPSRFSGSGSGTDFTLTISSLQPEDFATYYC | linker) |
| | | QQYYAYPWTFGQGTKVEIK | |
| | VH-CH1 | EVQLVESGGGLVQPGGSLRLSCAASGYTFTSYWLHWVRQAPGKGLEW | 182 |
| | | VGMIDPSNSDTRFNPNFKDRFTISADTSKNTAYLQMNSLRAEDTAVY | |
| | | YCATYRSYVTPLDYWGQGTLVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKKVEPKSCDK | |
| | VL-Ck | DIQMTQSPSSLSASVGDRVTITCKSSQSLLYTSSQKNYLAWYQQKPG | 183 |
| | | KAPKLLIYWASTRESGVPSRFSGSGSGTDFTLTISSLQPEDFATYYC | |
| | | QQYYAYPWTFGQGTKVEIKRTVAAPSVFIFPPSDEQLKSGTASVVCL | |
| | | LNNFYPREAKVQWKVDNALQSGNSQESVTEQDSKDSTYSLSSTLTLS | |
| | | KADYEKHKVYACEVTHQGLSSPVTKSFNRGEC | |
| CTAT.13 | VH | NYNMH | 184 |
| (CD47) | CDR1 | | |
| | VH | TIYPGNDDTSYNQKFKD | 185 |
| | CDR2 | | |
| | VH | GGYRAMDY | 186 |
| | CDR3 | | |
| | VH | QVQLVQSGAEVKKPGASVKVSCKASGYTFTNYNMHWVRQAPGQRLEW | 187 |
| | | MGTIYPGNDDTSYNQKFKDRVTITADTSASTAYMELSSLRSEDTAVY | |
| | | YCARGGYRAMDYWGQGTLVTVSS | |
| | VL | RSSQSIVYSNGNTYLG | 188 |
| | CDR1 | | |
| | VL | KVSNRFS | 189 |
| | CDR2 | | |
| | VL | FQGSHVPYT | 190 |
| | CDR3 | | |
| | VL | DIVMTQSPLSLPVTPGEPASISCRSSQSIVYSNGNTYLGWYLQKPGQ | 191 |
| | | SPQLLIYKVSNRFSGVPDRFSGSGSGTDFTLKISRVEAEDVGVYYCF | |
| | | QGSHVPYTFGQGTKLEIK | |
| | scFv | GGGSGGGQVQLVQSGAEVKKPGASVKVSCKASGYTFTNYNMHWVRQA | 192 |
| | | PGQRLEWMGTIYPGNDDTSYNQKFKDRVTITADTSASTAYMELSSLR | 268 (no N- |
| | | SEDTAVYYCARGGYRAMDYWGQGTLVTVSSGGGGSGGGGSGGGGSDI | terminus |
| | | VMTQSPLSLPVTPGEPASISCRSSQSIVYSNGNTYLGWYLQKPGQSP | peptide |
| | | QLLIYKVSNRFSGVPDRFSGSGSGTDFTLKISRVEAEDVGVYYCFQG | linker) |
| | | SHVPYTFGQGTKLEIK | |
| | VH-CH1 | QVQLVQSGAEVKKPGASVKVSCKASGYTFTNYNMHWVRQAPGQRLEW | 193 |
| | | MGTIYPGNDDTSYNQKFKDRVTITADTSASTAYMELSSLRSEDTAVY | |
| | | YCARGGYRAMDYWGQGTLVTVSSASTKGPSVFPLAPSSKSTSGGTAA | |
| | | LGCLVKDYFPEPVTVSWNSGALTSGVHTFPAVLQSSGLYSLSSVVTV | |
| | | PSSSLGTQTYICNVNHKPSNTKVDKRVEPKSCDK | |
| | VL-Ck | DIVMTQSPLSLPVTPGEPASISCRSSQSIVYSNGNTYLGWYLQKPGQ | 194 |
| | | SPQLLIYKVSNRFSGVPDRFSGSGSGTDFTLKISRVEAEDVGVYYCF | |
| | | QGSHVPYTFGQGTKLEIKRTVAAPSVFIFPPSDEQLKSGTASVVCLL | |
| | | NNFYPREAKVQWKVDNALQSGNSQESVTEQDSKDSTYSLSSTLTLSK | |
| | | ADYEKHKVYACEVTHQGLSSPVTKSFNRGEC | |
| CTAT.14 | VH | SGDYFWS | 195 |
| (TRAIL-R2) | CDR1 | | |
| | VH | HIHNSGTTYYNPSLKS | 196 |
| | CDR2 | | |
| | VH | DRGGDYYYGMDV | 197 |
| | CDR3 | | |
| | VH | QVQLQESGPGLVKPSQTLSLTCTVSGGSISSGDYFWSWIRQLPGKGL | 198 |
| | | EWIGHIHNSGTTYYNPSLKSRVTISVDTSKKQFSLRLSSVTAADTAV | |
| | | YYCARDRGGDYYYGMDVWGQGTTVTVSS | |
| | VL | RASQGISRSYLA | 199 |
| | CDR1 | | |
| | VL | GASSRAT | 200 |
| | CDR2 | | |
| | VL | QQFGSSPWT | 201 |
| | CDR3 | | |
| | VL | EIVLTQSPGTLSLSPGERATLSCRASQGISRSYLAWYQQKPGQAPSL | 202 |
| | | LIYGASSRATGIPDRFSGSGSGTDFTLTISRLEPEDFAVYYCQQFGS | |
| | | SPWTFGQGTKVEIK | |
| | scFv | GGGSGGGQVQLQESGPGLVKPSQTLSLTCTVSGGSISSGDYFWSWIR | 203 |
| | | QLPGKGLEWIGHIHNSGTTYYNPSLKSRVTISVDTSKKQFSLRLSSV | 269 (no N- |
| | | TAADTAVYYCARDRGGDYYYGMDVWGQGTTVTVSSGGGGSGGGGSGG | terminus |
| | | GGSEIVLTQSPGTLSLSPGERATLSCRASQGISRSYLAWYQQKPGQA | peptide |
| | | PSLLIYGASSRATGIPDRFSGSGSGTDFTLTISRLEPEDFAVYYCQQ | linker) |
| | | FGSSPWTFGQGTKVEIK | |
| | VH-CH1 | QVQLQESGPGLVKPSQTLSLTCTVSGGSISSGDYFWSWIRQLPGKGL | 204 |
| | | EWIGHIHNSGTTYYNPSLKSRVTISVDTSKKQFSLRLSSVTAADTAV | |
| | | YYCARDRGGDYYYGMDVWGQGTTVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDK | |
| | VL-Ck | EIVLTQSPGTLSLSPGERATLSCRASQGISRSYLAWYQQKPGQAPSL | 205 |
| | | LIYGASSRATGIPDRFSGSGSGTDFTLTISRLEPEDFAVYYCQQFGS | |
| | | SPWTFGQGTKVEIKRTVAAPSVFIFPPSDEQLKSGTASVVCLLNNFY | |
| | | PREAKVQWKVDNALQSGNSQESVTEQDSKDSTYSLSSTLTLSKADYE | |
| | | KHKVYACEVTHQGLSSPVTKSFNRGEC | |
| CTAT.15 | VH | SGSYYWS | 206 |
| (mesothelin) | CDR1 | | |
| | VH | YIYYSGSTNYNPSLKS | 207 |
| | CDR2 | | |
| | VH | EGENGAFDI | 208 |
| | CDR3 | | |
| | VH | QVQLQESGPGLVKPSETLSLTCTVSGGSVSSGSYYWSWIRQPPGKGL | 209 |
| | | EWIGYIYYSGSTNYNPSLKSRVTISVDTSKNQFSLKLSSVTAADTAV | |
| | | YYCAREGKNGAFDIWGQGTMVTVSS | |
| | VL | RASQSISSYLN | 210 |
| | CDR1 | | |
| | VL | AASSLQS | 211 |
| | CDR2 | | |
| | VL | QQSYSTPLT | 212 |
| | CDR3 | | |
| | VL | DIQMTQSPSSLSASVGDRVTITCRASQSISSYLNWYQQKPGKAPKLL | 213 |
| | | IYAASSLQSGVPSGFSGSGSGTDFTLTISSLQPEDFATYYCQQSYST | |
| | | PLTFGGGTKVEIK | |
| | scFv | GGGSGGGQVQLQESGPGLVKPSETLSLTCTVSGGSVSSGSYYWSWIR | 214 |
| | | QPPGKGLEWIGYIYYSGSTNYNPSLKSRVTISVDTSKNQFSLKLSSV | 270 (no N- |
| | | TAADTAVYYCAREGKNGAFDIWGQGTMVTVSSGGGGSGGGGSGGGGS | terminus |
| | | DIQMTQSPSSLSASVGDRVTITCRASQSISSYLNWYQQKPGKAPKLL | peptide |
| | | IYAASSLQSGVPSGFSGSGSGTDFTLTISSLQPEDFATYYCQQSYST | linker) |
| | | PLTFGGGTKVEIK | |
| | VH-CH1 | QVQLQESGPGLVKPSETLSLTCTVSGGSVSSGSYYWSWIRQPPGKGL | 215 |
| | | EWIGYIYYSGSTNYNPSLKSRVTISVDTSKNQFSLKLSSVTAADTAV | |
| | | YYCAREGKNGAFDIWGQGTMVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKKVEPKSCDK | |
| | VL-Ck | DIQMTQSPSSLSASVGDRVTITCRASQSISSYLNWYQQKPGKAPKLL | 216 |
| | | IYAASSLQSGVPSGFSGSGSGTDFTLTISSLQPEDFATYYCQQSYST | |
| | | PLTFGGGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CTAT.16 | VH | GYNMN | 217 |
| (GD2) | CDR1 | | |
| | VH | AIDPYYGGTSYNQKFKG | 218 |
| | CDR2 | | |
| | VH | GMEY | 219 |
| | CDR3 | | |
| | VH | EVQLLQSGPELEKPGASVMISCKASGSSFTGYNMNWVRQNIGKSLEW | 220 |
| | | IGAIDPYYGGTSYNQKFKGRATLTVDKSSSTAYMHLKSLTSEDSAVY | |
| | | YCVSGMEYWGQGTSVTVSS | |
| | VL | RSSQSLVHRNGNTYLH | 221 |
| | CDR1 | | |
| | VL | KVSNRFS | 222 |
| | CDR2 | | |
| | VL | SQSTHVPPLT | 223 |
| | CDR3 | | |
| | VL | EIVMTQSPATLSVSPGERATLSCRSSQSLVHRNGNTYLHWYLQKPGQ | 224 |
| | | SPKLLIHKVSNRFSGVPDRFSGSGSGTDFTLKISRVEAEDLGVYFCS | |
| | | QSTHVPPLTFGAGTKLELK | |
| | scFv | GGGSGGGEVQLLQSGPELEKPGASVMISCKASGSSFTGYNMNWVRQN | 225 |
| | | IGKSLEWIGAIDPYYGGTSYNQKFKGRATLTVDKSSSTAYMHLKSLT | 271 (no N- |
| | | SEDSAVYYCVSGMEYWGQGTSVTVSSGGGGSGGGGSGGGGSEIVMTQ | terminus |
| | | SPATLSVSPGERATLSCRSSQSLVHRNGNTYLHWYLQKPGQSPKLLI | peptide |
| | | HKVSNRFSGVPDRFSGSGSGTDFTLKISRVEAEDLGVYFCSQSTHVP | linker) |
| | | PLTFGAGTKLELK | |
| | VH-CH1 | EVQLLQSGPELEKPGASVMISCKASGSSFTGYNMNWVRQNIGKSLEW | 226 |
| | | IGAIDPYYGGTSYNQKFKGRATLTVDKSSSTAYMHLKSLTSEDSAVY | |
| | | YCVSGMEYWGQGTSVTVSSASTKGPSVFPLAPSSKSTSGGTAALGCL | |
| | | VKDYFPEPVTVSWNSGALTSGVHTFPAVLQSSGLYSLSSVVTVPSSS | |
| | | LGTQTYICNVNHKPSNTKVDKRVEPKSCDK | |
| | VL-Ck | EIVMTQSPATLSVSPGERATLSCRSSQSLVHRNGNTYLHWYLQKPGQ | 227 |
| | | SPKLLIHKVSNRFSGVPDRFSGSGSGTDFTLKISRVEAEDLGVYFCS | |
| | | QSTHVPPLTFGAGTKLELKRTVAAPSVFIFPPSDEQLKSGTASVVCL | |
| | | LNNFYPREAKVQWKVDNALQSGNSQESVTEQDSKDSTYSLSSTLTLS | |
| | | KADYEKHKVYACEVTHQGLSSPVTKSFNRGEC | |
| CTAT.17 | VH | SGDYYWT | 272 |
| (EGFR) | CDR1 | | |
| | VH | HIYYSGNTNYNPSLKS | 273 |
| | CDR2 | | |
| | VH | DRVTGAFDI | 274 |
| | CDR3 | | |
| | VH | QVQLQESGPGLVKPSETLSLTCTVSGGSVSSGDYYWTWIRQSPGKGL | 275 |
| | | EWIGHIYYSGNTNYNPSLKSRLTISIDTSKTQFSLKLSSVTAADTAI | |
| | | YYCVRDRVTGAFDIWGQGTMVTVSS | |
| | VL | QASQDISNYLN | 276 |
| | CDR1 | | |
| | VL | DASNLET | 277 |
| | CDR2 | | |
| | VL | QHFDHLPLA | 278 |
| | CDR3 | | |
| | VL | DIQMTQSPSSLSASVGDRVTITCQASQDISNYLNWYQQKPGKAPKLL | 279 |
| | | IYDASNLETGVPSRFSGSGSGTDFTFTISSLQPEDIATYFCQHEDHL | |
| | | PLAFGGGTKVEIKR | |
| | scFv | GGGSGGGQVQLQESGPGLVKPSETLSLTCTVSGGSVSSGDYYWTWIR | 280 |
| | | QSPGKGLEWIGHIYYSGNTNYNPSLKSRLTISIDTSKTQFSLKLSSV | 281 (no N- |
| | | TAADTAIYYCVRDRVTGAFDIWGQGTMVTVSSGGGGSGGGGSGGGGS | terminus |
| | | DIQMTQSPSSLSASVGDRVTITCQASQDISNYLNWYQQKPGKAPKLL | peptide |
| | | IYDASNLETGVPSRFSGSGSGTDFTFTISSLQPEDIATYFCQHFDHL | linker) |
| | | PLAFGGGTKVEIKR | |
| | VH-CH1 | QVQLQESGPGLVKPSETLSLTCTVSGGSVSSGDYYWTWIRQSPGKGL | 282 |
| | | EWIGHIYYSGNTNYNPSLKSRLTISIDTSKTQFSLKLSSVTAADTAI | |
| | | YYCVRDRVTGAFDIWGQGTMVTVSSASTKGPSVFPLAPSSKSTSGGT | |
| | | AALGCLVKDYFPEPVTVSWNSGALTSGVHTFPAVLQSSGLYSLSSVV | |
| | | TVPSSSLGTQTYICNVNHKPSNTKVDKRVEPKSCDK | |
| | VL-Ck | DIQMTQSPSSLSASVGDRVTITCQASQDISNYLNWYQQKPGKAPKLL | 283 |
| | | IYDASNLETGVPSRFSGSGSGTDFTFTISSLQPEDIATYFCQHFDHL | |
| | | PLAFGGGTKVEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |
| CTAT.18 | VH | SYNMH | 284 |
| (CD20) | CDR1 | | |
| | VH | AIYPGNGDTSYNQKFKG | 285 |
| | CDR2 | | |
| | VH | VVYYSNSYWYFDV | 286 |
| | CDR3 | | |
| | VH | EVQLVESGGGLVQPGGSLRLSCAASGYTFTSYNMHWVRQAPGKGLEW | 287 |
| | | VGAIYPGNGDTSYNQKFKGRFTISVDKSKNTLYLQMNSLRAEDTAVY | |
| | | YCARVVYYSNSYWYFDVWGQGTLVTVSS | |
| | VL | RASSSVSYMH | 288 |
| | CDR1 | | |
| | VL | APSNLAS | 289 |
| | CDR2 | | |
| | VL | QQWSFNPPT | 290 |
| | CDR3 | | |
| | VL | DIQMTQSPSSLSASVGDRVTITCRASSSVSYMHWYQQKPGKAPKPLI | 291 |
| | | YAPSNLASGVPSRFSGSGSGTDFTLTISSLQPEDFATYYCQQWSFNP | |
| | | PTFGQGTKVEIKR | |
| | scFv | GGGSGGGEVQLVESGGGLVQPGGSLRLSCAASGYTFTSYNMHWVRQA | 292 |
| | | PGKGLEWVGAIYPGNGDTSYNQKFKGRFTISVDKSKNTLYLQMNSLR | 293 (no N- |
| | | AEDTAVYYCARVVYYSNSYWYFDVWGQGTLVTVSSGGGGSGGGGSGG | terminus |
| | | GGSDIQMTQSPSSLSASVGDRVTITCRASSSVSYMHWYQQKPGKAPK | peptide |
| | | PLIYAPSNLASGVPSRFSGSGSGTDFTLTISSLQPEDFATYYCQQWS | linker) |
| | | FNPPTFGQGTKVEIKR | |
| | VH-CH1 | EVQLVESGGGLVQPGGSLRLSCAASGYTFTSYNMHWVRQAPGKGLEW | 294 |
| | | VGAIYPGNGDTSYNQKFKGRFTISVDKSKNTLYLQMNSLRAEDTAVY | |
| | | YCARVVYYSNSYWYFDVWGQGTLVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDK | |
| | VL-Ck | DIQMTQSPSSLSASVGDRVTITCRASSSVSYMHWYQQKPGKAPKPLI | 295 |
| | | YAPSNLASGVPSRFSGSGSGTDFTLTISSLQPEDFATYYCQQWSFNP | |
| | | PTFGQGTKVEIKRTVAAPSVFIFPPSDEQLKSGTASVVCLLNNFYPR | |
| | | EAKVQWKVDNALQSGNSQESVTEQDSKDSTYSLSSTLTLSKADYEKH | |
| | | KVYACEVTHQGLSSPVTKSFNRGEC | |
| CTAT.19 | VH | DYAMH | 296 |
| (CD20) | CDR1 | | |
| | VH | TISWNSGSIGYADSVKG | 297 |
| | CDR2 | | |
| | VH | DIQYGNYYYGMDV | 298 |
| | CDR3 | | |
| | VH | EVQLVESGGGLVQPGRSLRLSCAASGFTFNDYAMHWVRQAPGKGLEW | 299 |
| | | VSTISWNSGSIGYADSVKGRFTISRDNAKKSLYLQMNSLRAEDTALY | |
| | | YCAKDIQYGNYYYGMDVWGQGTTVTVSS | |
| | VL | RASQSVSSYLA | 300 |
| | CDR1 | | |
| | VL | DASNRAT | 301 |
| | CDR2 | | |
| | VL | QQRSNWPIT | 302 |
| | CDR3 | | |
| | VL | EIVLTQSPATLSLSPGERATLSCRASQSVSSYLAWYQQKPGQAPRLL | 303 |
| | | IYDASNRATGIPARFSGSGSGTDFTLTISSLEPEDFAVYYCQQRSNW | |
| | | PITFGQGTRLEIKR | |
| | scFv | GGGSGGGEVQLVESGGGLVQPGRSLRLSCAASGFTENDYAMHWVRQA | 304 |
| | | PGKGLEWVSTISWNSGSIGYADSVKGRFTISRDNAKKSLYLQMNSLR | |
| | | AEDTALYYCAKDIQYGNYYYGMDVWGQGTTVTVSSGGGGSGGGGSGG | |
| | | GGSEIVLTQSPATLSLSPGERATLSCRASQSVSSYLAWYQQKPGQAP | |
| | | RLLIYDASNRATGIPARFSGSGSGTDFTLTISSLEPEDFAVYYCQQR | |
| | | SNWPITFGQGTRLEIKR | |
| | VH-CH1 | EVQLVESGGGLVQPGRSLRLSCAASGFTENDYAMHWVRQAPGKGLEW | 305 |
| | | VSTISWNSGSIGYADSVKGRFTISRDNAKKSLYLQMNSLRAEDTALY | |
| | | YCAKDIQYGNYYYGMDVWGQGTTVTVSSASTKGPSVFPLAPSSKSTS | |
| | | GGTAALGCLVKDYFPEPVTVSWNSGALTSGVHTFPAVLQSSGLYSLS | |
| | | SVVTVPSSSLGTQTYICNVNHKPSNTKVDKRVEPKSCDK | |
| | VL-Ck | EIVLTQSPATLSLSPGERATLSCRASQSVSSYLAWYQQKPGQAPRLL | 306 |
| | | IYDASNRATGIPARFSGSGSGTDFTLTISSLEPEDFAVYYCQQRSNW | |
| | | PITFGQGTRLEIKRTVAAPSVFIFPPSDEQLKSGTASVVCLLNNFYP | |
| | | REAKVQWKVDNALQSGNSQESVTEQDSKDSTYSLSSTLTLSKADYEK | |
| | | HKVYACEVTHQGLSSPVTKSFNRGEC | |

In some examples, the anti-TAA binding moiety may comprise the same heavy chain CDRs as those in antibody CTAT.01, which are provided in Table 2 above. Alternatively or in addition, the anti-TAA binding moiety may have the same light chain CDRs as those in antibody CTAT.01, which are also provided in Table 2 above. Such an anti-TAA binding moiety may comprise the same V_{H} and/or V_{L} chains as CTAT.01. Alternatively, the anti-TAA binding moiety may comprise amino acid variations in one or more of the framework regions relative to the corresponding framework regions in CTAT.01. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.01.

In some embodiments, the anti-TAA moiety may comprise a certain level of variations in one or more of the CDRs relative to those of CTAT.01. For example, the anti-TAA moiety may comprise heavy chain CDRs that are at least 80% (e.g., 85%, 90%, 95%, or 98%) sequence identity, individually or collectively, as compared with the V_{H} CDRs of CTAT.01. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.01.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.01. In some instances, the anti-TAA moiety may comprise the same heavy chain CDR3 as the heavy chain CDR3 of CTAT.01 and comprise one or more amino acid variations in one or more of the other heavy chain and light chain CDRs.

In some examples, the anti-TAA binding moiety may comprise the same heavy chain CDRs as those in antibody CTAT.02, which are provided in Table 2 above. Alternatively or in addition, the anti-TAA binding moiety may have the same light chain CDRs as those in antibody CTAT.02, which are also provided in Table 2 above. Such an anti-TAA binding moiety may comprise the same V_{H} and/or V_{L} chains as CTAT.02. Alternatively, the anti-TAA binding moiety may comprise amino acid variations in one or more of the framework regions relative to the corresponding framework regions in CTAT.02. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.02.

In one specific example, the anti-TAA moiety disclosed herein comprises a mutation at position G42 of the VL chain relative to CTAT.02, for example, an amino acid residue substitution (e.g., G42A). See, e.g., CTAT.02 VL-01 in Table 2 above. In another specific example, the anti-TAA moiety disclosed herein comprises a mutation at position D41 of the VL chain relative to CTAT.02, for example, an amino acid residue substitution (e.g., D41E). See, e.g., CTAT.02 VL-02 in Table 2 above.

In some embodiments, the anti-TAA moiety may comprise a certain level of variations in one or more of the CDRs relative to those of CTAT.02. For example, the anti-TAA moiety may comprise heavy chain CDRs that are at least 80% (e.g., 85%, 90%, 95%, or 98%) sequence identity, individually or collectively, as compared with the V_{H} CDRs of CTAT.02. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.02.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.02. In some instances, the anti-TAA moiety may comprise the same heavy chain CDR3 as the heavy chain CDR3 of CTAT.02 and comprise one or more amino acid variations in one or more of the other heavy chain and light chain CDRs.

In some examples, the anti-TAA binding moiety may comprise the same heavy chain CDRs as those in antibody CTAT.03, which are provided in Table 2 above. Alternatively or in addition, the anti-TAA binding moiety may have the same light chain CDRs as those in antibody CTAT.03, which are also provided in Table 2 above. Such an anti-TAA binding moiety may comprise the same V_{H} and/or V_{L} chains as CTAT.03. Alternatively, the anti-TAA binding moiety may comprise amino acid variations in one or more of the framework regions relative to the corresponding framework regions in CTAT.03. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.03.

In some embodiments, the anti-TAA moiety may comprise a certain level of variations in one or more of the CDRs relative to those of CTAT.03. For example, the anti-TAA moiety may comprise heavy chain CDRs that are at least 80% (e.g., 85%, 90%, 95%, or 98%) sequence identity, individually or collectively, as compared with the V_{H} CDRs of CTAT.03. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.03.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.03. In some instances, the anti-TAA moiety may comprise the same heavy chain CDR3 as the heavy chain CDR3 of CTAT.03 and comprise one or more amino acid variations in one or more of the other heavy chain and light chain CDRs.

In some examples, the anti-TAA binding moiety may comprise the same heavy chain CDRs as those in antibody CTAT.04, which are provided in Table 2 above. Alternatively or in addition, the anti-TAA binding moiety may have the same light chain CDRs as those in antibody CTAT.04, which are also provided in Table 2 above. Such an anti-TAA binding moiety may comprise the same V_{H} and/or V_{L} chains as CTAT.04. Alternatively, the anti-TAA binding moiety may comprise amino acid variations in one or more of the framework regions relative to the corresponding framework regions in CTAT.04. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.04.

In some embodiments, the anti-TAA moiety may comprise a certain level of variations in one or more of the CDRs relative to those of CTAT.04. For example, the anti-TAA moiety may comprise heavy chain CDRs that are at least 80% (e.g., 85%, 90%, 95%, or 98%) sequence identity, individually or collectively, as compared with the V_{H} CDRs of CTAT.04. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.04.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.04. In some instances, the anti-TAA moiety may comprise the same heavy chain CDR3 as the heavy chain CDR3 of CTAT.04 and comprise one or more amino acid variations in one or more of the other heavy chain and light chain CDRs.

In some examples, the anti-TAA binding moiety may comprise the same heavy chain CDRs as those in antibody CTAT.05, which are provided in Table 2 above. Alternatively or in addition, the anti-TAA binding moiety may have the same light chain CDRs as those in antibody CTAT.05, which are also provided in Table 2 above. Such an anti-TAA binding moiety may comprise the same V_{H} and/or V_{L} chains as CTAT.05. Alternatively, the anti-TAA binding moiety may comprise amino acid variations in one or more of the framework regions relative to the corresponding framework regions in CTAT.05. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.05.

In some embodiments, the anti-TAA moiety may comprise a certain level of variations in one or more of the CDRs relative to those of CTAT.05. For example, the anti-TAA moiety may comprise heavy chain CDRs that are at least 80% (e.g., 85%, 90%, 95%, or 98%) sequence identity, individually or collectively, as compared with the V_{H} CDRs of CTAT.05. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.05.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.05. In some instances, the anti-TAA moiety may comprise the same heavy chain CDR3 as the heavy chain CDR3 of CTAT.05 and comprise one or more amino acid variations in one or more of the other heavy chain and light chain CDRs.

In some examples, the anti-TAA binding moiety may comprise the same heavy chain CDRs as those in antibody CTAT.06, which are provided in Table 2 above. Alternatively or in addition, the anti-TAA binding moiety may have the same light chain CDRs as those in antibody CTAT.06, which are also provided in Table 2 above. Such an anti-TAA binding moiety may comprise the same V_{H} and/or V_{L} chains as CTAT.06. Alternatively, the anti-TAA binding moiety may comprise amino acid variations in one or more of the framework regions relative to the corresponding framework regions in CTAT.06. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.06.

In some embodiments, the anti-TAA moiety may comprise a certain level of variations in one or more of the CDRs relative to those of CTAT.06. For example, the anti-TAA moiety may comprise heavy chain CDRs that are at least 80% (e.g., 85%, 90%, 95%, or 98%) sequence identity, individually or collectively, as compared with the V_{H} CDRs of CTAT.06. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.06.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.06. In some instances, the anti-TAA moiety may comprise the same heavy chain CDR3 as the heavy chain CDR3 of CTAT.06 and comprise one or more amino acid variations in one or more of the other heavy chain and light chain CDRs.

In some examples, the anti-TAA binding moiety may comprise the same heavy chain CDRs as those in antibody CTAT.07, which are provided in Table 2 above. Alternatively or in addition, the anti-TAA binding moiety may have the same light chain CDRs as those in antibody CTAT.07, which are also provided in Table 2 above. Such an anti-TAA binding moiety may comprise the same V_{H} and/or V_{L} chains as CTAT.07. Alternatively, the anti-TAA binding moiety may comprise amino acid variations in one or more of the framework regions relative to the corresponding framework regions in CTAT.07. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.07.

In some embodiments, the anti-TAA moiety may comprise a certain level of variations in one or more of the CDRs relative to those of CTAT.07. For example, the anti-TAA moiety may comprise heavy chain CDRs that are at least 80% (e.g., 85%, 90%, 95%, or 98%) sequence identity, individually or collectively, as compared with the V_{H} CDRs of CTAT.07. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.07.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.07. In some instances, the anti-TAA moiety may comprise the same heavy chain CDR3 as the heavy chain CDR3 of CTAT.07 and comprise one or more amino acid variations in one or more of the other heavy chain and light chain CDRs.

In some examples, the anti-TAA binding moiety may comprise the same heavy chain CDRs as those in antibody CTAT.08, which are provided in Table 2 above. Alternatively or in addition, the anti-TAA binding moiety may have the same light chain CDRs as those in antibody CTAT.08, which are also provided in Table 2 above. Such an anti-TAA binding moiety may comprise the same V_{H} and/or V_{L} chains as CTAT.08. Alternatively, the anti-TAA binding moiety may comprise amino acid variations in one or more of the framework regions relative to the corresponding framework regions in CTAT.08. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.08.

In some embodiments, the anti-TAA moiety may comprise a certain level of variations in one or more of the CDRs relative to those of CTAT.08. For example, the anti-TAA moiety may comprise heavy chain CDRs that are at least 80% (e.g., 85%, 90%, 95%, or 98%) sequence identity, individually or collectively, as compared with the V_{H} CDRs of CTAT.08. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.08.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.08. In some instances, the anti-TAA moiety may comprise the same heavy chain CDR3 as the heavy chain CDR3 of CTAT.08 and comprise one or more amino acid variations in one or more of the other heavy chain and light chain CDRs.

In some examples, the anti-TAA binding moiety may comprise the same heavy chain CDRs as those in antibody CTAT.09, which are provided in Table 2 above. Alternatively or in addition, the anti-TAA binding moiety may have the same light chain CDRs as those in antibody CTAT.09, which are also provided in Table 2 above. Such an anti-TAA binding moiety may comprise the same V_{H} and/or V_{L} chains as CTAT.09. Alternatively, the anti-TAA binding moiety may comprise amino acid variations in one or more of the framework regions relative to the corresponding framework regions in CTAT.09. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.09.

In some embodiments, the anti-TAA moiety may comprise a certain level of variations in one or more of the CDRs relative to those of CTAT.09. For example, the anti-TAA moiety may comprise heavy chain CDRs that are at least 80% (e.g., 85%, 90%, 95%, or 98%) sequence identity, individually or collectively, as compared with the V_{H} CDRs of CTAT.09. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.09.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.09. In some instances, the anti-TAA moiety may comprise the same heavy chain CDR3 as the heavy chain CDR3 of CTAT.09 and comprise one or more amino acid variations in one or more of the other heavy chain and light chain CDRs.

In some examples, the anti-TAA binding moiety may comprise the same heavy chain CDRs as those in antibody CTAT.10, which are provided in Table 2 above. Alternatively or in addition, the anti-TAA binding moiety may have the same light chain CDRs as those in antibody CTAT.10, which are also provided in Table 2 above. Such an anti-TAA binding moiety may comprise the same V_{H} and/or V_{L} chains as CTAT.10. Alternatively, the anti-TAA binding moiety may comprise amino acid variations in one or more of the framework regions relative to the corresponding framework regions in CTAT.10. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.10.

In some embodiments, the anti-TAA moiety may comprise a certain level of variations in one or more of the CDRs relative to those of CTAT.10. For example, the anti-TAA moiety may comprise heavy chain CDRs that are at least 80% (e.g., 85%, 90%, 95%, or 98%) sequence identity, individually or collectively, as compared with the V_{H} CDRs of CTAT.10. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.10.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.10. In some instances, the anti-TAA moiety may comprise the same heavy chain CDR3 as the heavy chain CDR3 of CTAT.10 and comprise one or more amino acid variations in one or more of the other heavy chain and light chain CDRs.

In some examples, the anti-TAA binding moiety may comprise the same heavy chain CDRs as those in antibody CTAT.11, which are provided in Table 2 above. Alternatively or in addition, the anti-TAA binding moiety may have the same light chain CDRs as those in antibody CTAT.11, which are also provided in Table 2 above. Such an anti-TAA binding moiety may comprise the same V_{H} and/or V_{L} chains as CTAT.11. Alternatively, the anti-TAA binding moiety may comprise amino acid variations in one or more of the framework regions relative to the corresponding framework regions in CTAT.11. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.11.

In some embodiments, the anti-TAA moiety may comprise a certain level of variations in one or more of the CDRs relative to those of CTAT.11. For example, the anti-TAA moiety may comprise heavy chain CDRs that are at least 80% (e.g., 85%, 90%, 95%, or 98%) sequence identity, individually or collectively, as compared with the V_{H} CDRs of CTAT.11. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.10.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.11. In some instances, the anti-TAA moiety may comprise the same heavy chain CDR3 as the heavy chain CDR3 of CTAT.11 and comprise one or more amino acid variations in one or more of the other heavy chain and light chain CDRs.

In some examples, the anti-TAA binding moiety may comprise the same heavy chain CDRs as those in antibody CTAT.12, which are provided in Table 2 above. Alternatively or in addition, the anti-TAA binding moiety may have the same light chain CDRs as those in antibody CTAT.12, which are also provided in Table 2 above. Such an anti-TAA binding moiety may comprise the same V_{H} and/or V_{L} chains as CTAT.12. Alternatively, the anti-TAA binding moiety may comprise amino acid variations in one or more of the framework regions relative to the corresponding framework regions in CTAT.12. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.12.

In some embodiments, the anti-TAA moiety may comprise a certain level of variations in one or more of the CDRs relative to those of CTAT.12. For example, the anti-TAA moiety may comprise heavy chain CDRs that are at least 80% (e.g., 85%, 90%, 95%, or 98%) sequence identity, individually or collectively, as compared with the V_{H} CDRs of CTAT.12. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.12.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.12. In some instances, the anti-TAA moiety may comprise the same heavy chain CDR3 as the heavy chain CDR3 of CTAT.12 and comprise one or more amino acid variations in one or more of the other heavy chain and light chain CDRs.

In some examples, the anti-TAA binding moiety may comprise the same heavy chain CDRs as those in antibody CTAT.13, which are provided in Table 2 above. Alternatively or in addition, the anti-TAA binding moiety may have the same light chain CDRs as those in antibody CTAT.13, which are also provided in Table 2 above. Such an anti-TAA binding moiety may comprise the same V_{H} and/or V_{L} chains as CTAT.13. Alternatively, the anti-TAA binding moiety may comprise amino acid variations in one or more of the framework regions relative to the corresponding framework regions in CTAT.13. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.13.

In some embodiments, the anti-TAA moiety may comprise a certain level of variations in one or more of the CDRs relative to those of CTAT.13. For example, the anti-TAA moiety may comprise heavy chain CDRs that are at least 80% (e.g., 85%, 90%, 95%, or 98%) sequence identity, individually or collectively, as compared with the V_{H} CDRs of CTAT.13. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.13.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.13. In some instances, the anti-TAA moiety may comprise the same heavy chain CDR3 as the heavy chain CDR3 of CTAT.13 and comprise one or more amino acid variations in one or more of the other heavy chain and light chain CDRs.

In some examples, the anti-TAA binding moiety may comprise the same heavy chain CDRs as those in antibody CTAT.14, which are provided in Table 2 above. Alternatively or in addition, the anti-TAA binding moiety may have the same light chain CDRs as those in antibody CTAT.14, which are also provided in Table 2 above. Such an anti-TAA binding moiety may comprise the same V_{H} and/or V_{L} chains as CTAT.14. Alternatively, the anti-TAA binding moiety may comprise amino acid variations in one or more of the framework regions relative to the corresponding framework regions in CTAT.14. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.14.

In some embodiments, the anti-TAA moiety may comprise a certain level of variations in one or more of the CDRs relative to those of CTAT.14. For example, the anti-TAA moiety may comprise heavy chain CDRs that are at least 80% (e.g., 85%, 90%, 95%, or 98%) sequence identity, individually or collectively, as compared with the V_{H} CDRs of CTAT.14. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.14.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.14. In some instances, the anti-TAA moiety may comprise the same heavy chain CDR3 as the heavy chain CDR3 of CTAT.14 and comprise one or more amino acid variations in one or more of the other heavy chain and light chain CDRs.

In some examples, the anti-TAA binding moiety may comprise the same heavy chain CDRs as those in antibody CTAT.15, which are provided in Table 2 above. Alternatively or in addition, the anti-TAA binding moiety may have the same light chain CDRs as those in antibody CTAT.15, which are also provided in Table 2 above. Such an anti-TAA binding moiety may comprise the same V_{H} and/or V_{L} chains as CTAT.15. Alternatively, the anti-TAA binding moiety may comprise amino acid variations in one or more of the framework regions relative to the corresponding framework regions in CTAT.15. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.15.

In some embodiments, the anti-TAA moiety may comprise a certain level of variations in one or more of the CDRs relative to those of CTAT.15. For example, the anti-TAA moiety may comprise heavy chain CDRs that are at least 80% (e.g., 85%, 90%, 95%, or 98%) sequence identity, individually or collectively, as compared with the V_{H} CDRs of CTAT.15. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.15.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.15. In some instances, the anti-TAA moiety may comprise the same heavy chain CDR3 as the heavy chain CDR3 of CTAT.15 and comprise one or more amino acid variations in one or more of the other heavy chain and light chain CDRs.

In some examples, the anti-TAA binding moiety may comprise the same heavy chain CDRs as those in antibody CTAT.16, which are provided in Table 2 above. Alternatively or in addition, the anti-TAA binding moiety may have the same light chain CDRs as those in antibody CTAT.16, which are also provided in Table 2 above. Such an anti-TAA binding moiety may comprise the same V_{H} and/or V_{L} chains as CTAT.16. Alternatively, the anti-TAA binding moiety may comprise amino acid variations in one or more of the framework regions relative to the corresponding framework regions in CTAT.16. For example, the anti-TAA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in CTAT.16.

In some embodiments, the anti-TAA moiety may comprise a certain level of variations in one or more of the CDRs relative to those of CTAT.16. For example, the anti-TAA moiety may comprise heavy chain CDRs that are at least 80% (e.g., 85%, 90%, 95%, or 98%) sequence identity, individually or collectively, as compared with the V_{H} CDRs of CTAT.16. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.16.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.16. In some instances, the anti-TAA moiety may comprise the same heavy chain CDR3 as the heavy chain CDR3 of CTAT.16 and comprise one or more amino acid variations in one or more of the other heavy chain and light chain CDRs.

In some embodiments, the anti-TAA moiety may comprise a certain level of variations in one or more of the CDRs relative to those of CTAT.17. For example, the anti-TAA moiety may comprise heavy chain CDRs that are at least 80% (e.g., 85%, 90%, 95%, or 98%) sequence identity, individually or collectively, as compared with the V_{H} CDRs of CTAT.17. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.17.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.17. In some instances, the anti-TAA moiety may comprise the same heavy chain CDR3 as the heavy chain CDR3 of CTAT.17 and comprise one or more amino acid variations in one or more of the other heavy chain and light chain CDRs.

In some embodiments, the anti-TAA moiety may comprise a certain level of variations in one or more of the CDRs relative to those of CTAT.18. For example, the anti-TAA moiety may comprise heavy chain CDRs that are at least 80% (e.g., 85%, 90%, 95%, or 98%) sequence identity, individually or collectively, as compared with the V_{H} CDRs of CTAT.18. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.18.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.18. In some instances, the anti-TAA moiety may comprise the same heavy chain CDR3 as the heavy chain CDR3 of CTAT.18 and comprise one or more amino acid variations in one or more of the other heavy chain and light chain CDRs.

In some embodiments, the anti-TAA moiety may comprise a certain level of variations in one or more of the CDRs relative to those of CTAT.19. For example, the anti-TAA moiety may comprise heavy chain CDRs that are at least 80% (e.g., 85%, 90%, 95%, or 98%) sequence identity, individually or collectively, as compared with the V_{H} CDRs of CTAT.19. Alternatively or in addition, the anti-TAA antibody may comprise light chain CDRs that are at least 80% (e.g., 85%, 90%, 95%, or 98%) sequence identity, individually or collectively, as compared with the V_{L} CDRs as CTAT.19.

In some instances, the anti-TAA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of CTAT.19. In some instances, the anti-TAA moiety may comprise the same heavy chain CDR3 as the heavy chain CDR3 of CTAT.19 and comprise one or more amino acid variations in one or more of the other heavy chain and light chain CDRs.

(iii) Anti-CD3/Anti-TAA Bispecific Antibodies

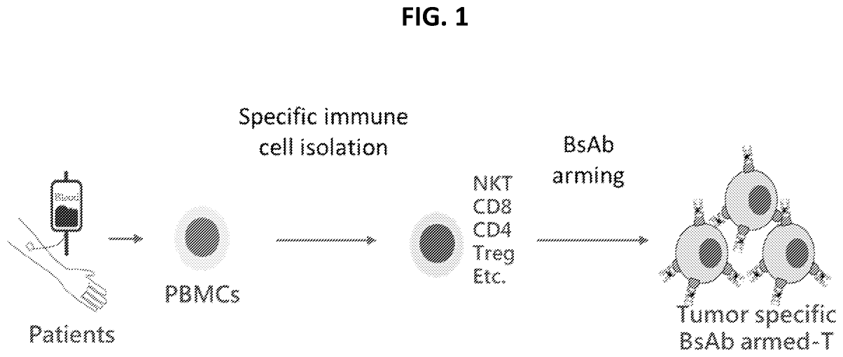

The bispecific antibody disclosed herein may be in any suitable format as those known in the art, for example, those disclosed in Mol. Immunol. 67(2):95-106 (2015), the relevant disclosures of which are incorporated by reference for the subject matter and purpose referenced herein. Some examples are provided below. See also FIGS. 1A-1N.

In some embodiments, the bispecific antibody disclosed herein may comprise one antigen binding moiety in Fab format and the other antigen binding moiety in single chain variable fragment (scFv) format. Such a bispecific antibody may comprise two polypeptides, one comprising the heavy or light chain of the Fab fragment linked to the scFv fragment and the other comprising the light or heavy chain of the Fab that is not linked to the scFv fragment.

In some instances, a Fab fragment comprises two polypeptide chains, one comprising a VH domain linked to a fragment of a heavy chain constant region (e.g., CH1) and the other one comprising a VL domain linked to a light chain constant region. The heavy chain constant region fragment may be from any Ig subclass, for example, IgG, IgA, IgE, IgD, or IgM. In some examples, the heavy chain constant region fragment is from an IgG molecule (e.g., a human IgG molecule). The light chain constant region may be a kappa chain or a lambda chain (e.g., a human kappa or lambda chain). An scFv fragment comprises a VH domain and a VL domain linked by a peptide linker. See e.g., Bird et al. (1988) Science 242:423-426; and Huston et al. (1988) Proc. Natl. Acad. Sci. USA 85:5879-5883. In some instances, the scFv fragment has, form N-terminus to C-terminus, the VH-linker-VL orientation. Alternatively, the scFv fragment has, form N-terminus to C-terminus, the VL-linker-VH orientation. In the bispecific antibody, the scFv fragment may be linked to the heavy chain of the Fab fragment. Alternatively, the scFv may be linked to the light chain of the Fab fragment. See, e.g., WO2021/195067, the relevant disclosures of which are incorporated by reference for the subject matter and purpose referenced herein.

In some examples, the bispecific antibody disclosed herein may comprise the anti-CD3 binding moiety in Fab format and the anti-TAA binding moiety in scFv format. The anti-CD3 Fab comprises a heavy chain VH-CH1 domain and a light chain VL-Cκ or VL-Cλ domain. The anti-TAA scFv comprises a VH domain and a VL domain. In some instances, the anti-CD3 Fab may be linked to the anti-TAA scFv via a peptide linker disposed between the CH1 domain of the anti-CD3 Fab heavy chain and the VH domain of the anti-tumor scFv. In other instances, the CH1 domain of the anti-CD3 Fab heavy chain can be linked to the VL domain of the anti-tumor scFv. In other instances, the anti-TAA scFv can be linked to the Cκ or Cλ domain of the anti-CD3 Fab light chain via the VL domain of the scFv, or via the VH domain of the anti-tumor scFv. Examples of anti-CD3 Fab heavy chain (VH-CH1) and light chains (VL-Ck) and examples of anti-TAA scFv fragments are provided in Tables 1 and 2, respectively. Any combination of such is within the scope of the present disclosure. Exemplary designs of such bispecific antibodies disclosed herein include those depicted in WO2021/195067 (e.g., FIGS. 1A-1D), the relevant disclosures of which are incorporated by reference for the subject matter and purpose referenced herein.

In some examples, the bispecific antibody disclosed herein may comprise the anti-TAA binding moiety in Fab format and the anti-CD3 binding moiety in scFv format. The anti-TAA Fab comprises a heavy chain VH-CH1 domain and a light chain VL-Cκ or VL-Cλ domain. The anti-CD3 scFv comprises a VH domain and a VL domain. In some instances, the anti-TAA Fab may be linked to the anti-CD3 scFv via a peptide linker disposed between the CH1 domain of the anti-TAA Fab heavy chain and the VH domain of the anti-CD3 scFv. In other instances, the CH1 domain of the anti-TAA Fab heavy chain can be linked to the VL domain of the anti-CD3 scFv. In other instances, the anti-CD3 scFv can be linked to the Cκ or Cλ domain of the anti-TAA Fab light chain via the VL domain of the scFv, or via the VH domain of the anti-CD3 scFv. Examples of anti-TAA Fab heavy chain (VH-CH1) and light chains (VL-Ck) and examples of anti-CD3 scFv fragments are provided in Tables 2 and 1, respectively. Any combination of such is within the scope of the present disclosure. Exemplary designs of such bispecific antibodies disclosed herein include those depicted in WO2021/195067 (e.g., FIGS. 1E-1H), the relevant disclosures of which are incorporated by reference for the subject matter and purpose referenced herein.

In some embodiments, the bispecific antibody disclosed herein may comprise both antigen binding moieties in scFv format. Exemplary designs of such bispecific antibodies disclosed herein include those depicted in WO2021/195067 (e.g., FIGS. 1I-1L), the relevant disclosures of which are incorporated by reference for the subject matter and purpose referenced herein.

In some examples, the VH domain of anti-CD3 scFv may be linked to the VH domain of the anti-TAA scFv via a peptide linker. In some examples, the VH domain of anti-CD3 scFv may be linked to the VL domain of the anti-TAA scFv via a peptide linker. In some examples, the VL domain of anti-CD3 scFv may be linked to the VH domain of the anti-TAA scFv via a peptide linker. In other examples, the VL domain of anti-CD3 scFv may be linked to the VH domain of the anti-TAA scFv via a peptide linker. Exemplary anti-CD3 scFv fragments and exemplary anti-TAA scFv fragments are provided in Tables 1 and 2, respectively. Any combination thereof for constructing a bispecific antibody is within the scope of the present disclosure.

In yet other embodiments, the bispecific antibodies disclosed herein may comprise one or more Fc regions, which may optionally a “knob into hole” structure, in which a knob in the CH2 domain, the CH3 domain, or both of the first heavy chain is created by replacing several amino acid side chains with alternative ones, and a hole in the juxtaposed position at the CH3 domain of the second heavy chain is created by replacing appropriate amino acid side chains with alternative ones. See, e.g., WO2021/195067 (e.g., FIGS. 1M and 1N), the relevant disclosures of which are incorporated by reference for the subject matter and purpose referenced herein.

Typically, the terms “a knob and a hole” or “knobs-into-holes” are used interchangeably herein. Knobs-into-holes amino acid changes is a rational design strategy known in the art for heterodimerization of the heavy (H) chains in the production of bispecific IgG antibodies. Carter, J. Immunol. Methods, 248(1-2):7-15 (2001), the relevant disclosures of which are incorporated by reference herein for the purpose and subject matter referenced herein.

In one example, the “knobs-into-holes” provides an approach as described in, e.g., Ridgway J B B et al., (1996) Protein Engineering, 9(7): 617-21 and U.S. Pat. No. 5,731,168, the relevant disclosures of each of which are incorporated by reference herein for the purpose and subject matter referenced herein. This approach has been shown to promote the formation of heterodimers of the first polypeptide and the second polypeptide chain, and hinder the assembly of corresponding homodimers. In one aspect, a knob is created by replacing small amino side chains at the interface between CH3 domains with larger ones, whereas a hole is constructed by replacing large side chains with smaller ones. In a specific example, the “knob” mutation comprises T366W and the “hole” mutations comprise T366S, L368A and Y407V (Atwell S et al., (1997) J. Mol. Biol. 270: 26-35).

In some instances, the bispecific antibody may comprise an anti-CD3 binding moiety comprising a first VH-CH1-CH2-CH3 domain and a first VL-Cκ or VL-Cλ domain, and an anti-TAA binding moiety comprising a second VH-CH1-CH2-CH3 domain and second a V_{L}-Cκ or VL-Cλ domain. The CH2 and/or CH3 in the heavy chain of the anti-CD3 binding moiety that those in the heavy chain of the anti-TAA binding moiety may comprise the knob/hole modifications, allowing for the binding between the two heavy chains. In other instances, the bispecific antibody may comprise an anti-Cd3 binding moiety comprising a first VH-CH1-CH2-CH3 domain and a first VL-Cκ or VL-Cλ domain, and an anti-TAA scFv linked to a second CH2-CH3 domain. The CH2 and/or CH3 in the heavy chain of the anti-CD3 binding moiety that those in the anti-TAA binding moiety may comprise the knob/hole modifications, allowing for the binding between the two heavy chains. In this setting, the format of the anti-CD3 binding moiety and the format of the anti-TAA binding moiety may be switched.

The term “peptide linker” refers to a peptide having natural or synthetic amino acid residues for connecting two polypeptides. For example, the peptide linker may be used to connect one V_{H} domain and one V_{L} domain to form a single chain variable fragment (e.g., scFv); to connect one scFv and one Fab to form a scFv/Fab recombinant antibody; to connect two scFvs to form a scFv/scFv recombinant antibody; or to connect two monovalent antibodies (e.g., two monovalent IgGs), two monovalent antibody fragments (e.g., two monovalent scFv-Fc fusion proteins), or one monovalent antibody and one monovalent antibody fragment (e.g., one monovalent IgG and on monovalent scFv-Fc fusion protein) thereby forming a divalent antibody. Preferably, the peptide linker is a peptide having at least 5 amino acid residues in length, such as 5 to 100 amino acid residues in length; more preferably, 10 to 30 amino acid residues in length. The peptide linker within scFv is a peptide of at least 5 amino acid residues in length, preferably 15 to 20 amino acid residues in length. Preferably, the peptide linker comprises a sequence of (GₙS)ₘ, with G=glycine, S=serine, and n and m are independently a number between 1 to 4. In one example, the linker comprises a sequence of (G₂S)₄. In another example, the linker comprises a sequence or (G₄S)₃.

The peptide linker for linking the first antibody fragment (i.e., anti-CD3 antibody fragment) and the second antibody fragment (i.e., anti-TAA antibody fragment) may be any peptide suitable for connecting two polypeptides. According to certain embodiments of the present disclosure, the peptide linker is a peptide having at least 5 amino acid residues in length, for example, having 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more amino acid residues in length. Preferably, the peptide linker of the present recombinant antibody consists of 10 to 30 glycine (G) and/or serine (S) residues.

In some embodiments, the bispecific antibodies described herein specifically bind to one or both of the corresponding target antigen (CD3 and a TAA) or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (CD3 and/or a TAA) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, a bispecific antibody as described herein has a suitable binding affinity for one or both of the target antigens (e.g., CD3 and a TAA) or antigenic epitopes thereof. As used herein, “binding affinity” refers to the apparent association constant or K_{A}. The K_{A} is the reciprocal of the dissociation constant (K_{D}). The bispecific antibody described herein may have a binding affinity (K_{D}) of at least 100 nM, 10 nM, lnM, 0.1 nM, or lower for CD3 (e.g., lower than 1 nM or 0.1 nM). Alternatively, the bispecific antibody described herein may have a binding affinity (K_{D}) of at least 100 nM, 10 nM, lnM, 0.1 nM, or lower for the TAA.

An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-CD3 and/or anti-TAA antibodies for making the bispecific antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

Exemplary bispecific antibodies as disclosed herein are provided in Table 3 below (using anti-CD3 binding moieties from CTA.03 as examples). Anti-CD3 binding moieties from other anti-CD3 reference antibodies (e.g., CTA.02, CTA.04, and CTA.05) are also within the scope of the present disclosure.

TABLE 3
Exemplary Bispecific Antibodies

| Bi-Specific Antibodies | | Amino Acid Sequences |
|---|---|---|
| CTA.03Fab/ | 1^{st} chain | EVQLLESGGGLVQPGGSLRLSCAASGFTFSSFPMAWVRQAPGKGLEWVSTISTS |
| CTAT.01scFv | | GGRTYYRDSVKGRFTISRDNSKNTLYLQMNSLRAEDTAVYYCAKFRQYSGGFDY |
| | | WGQGTLVTVSSASTKGPSVEPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQQPGAELVKPGASVKMSCKASGYTFTSYNMHWVKQTP |
| | | GRGLEWIGAIYPGNGDTSYNQKFKGKATLTADKSSSTAYMQLSSLTSEDSAVYY |
| | | CARSTYYGGDWYFNVWGAGTTVTVSAGGGGSGGGGSGGGGSQIVLSQSPAILSA |
| | | SPGEKVTMTCRASSSVSYIHWFQQKPGSSPKPWIYATSNLASGVPVRFSGSGSG |
| | | TSYSLTISRVEAEDAATYYCQQWTSNPPTFGGGTKLEIK (SEQ ID NO: |
| | | 229) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.02scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDGTVKLLIYHTSRLHSGVPSRFSGSGSG |
| | | TDYSLTISNLEQEDIATYFCQQGNTLPYTFGGGTKLEIT (SEQ ID NO: |
| | | 230) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.03scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLKQSGPGLVQPSQSLSITCTVSGESLINYGVHWVRQSP |
| | | GKGLEWLGVIWSGGNTDYNTPFTSRLSINKDNSKSQVFFKMNSLQSNDTAIYYC |
| | | ARALTYYDYEFAYWGQGTLVTVSAGGGGSGGGGSGGGGSDILLTQSPVILSVSP |
| | | GERVSFSCRASQSIGTNIHWYQQRINGSPRLLIKYASESISGIPSRFSGSGSGT |
| | | DFTLSINSVESEDIADYYCQQNNNWPTTFGAGTKLELK (SEQ ID NO: |
| | | 231) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.04scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGGSLRLSCAASGFNIKDTYIHWVRQAP |
| | | GKGLEWVARIYPTNGYTRYADSVKGRFTISADTSKNTAYLQMNSLRAEDTAVYY |
| | | CSRWGGDGFYAMDYWGQGTLVTVSSGGGGSGGGGSGGGGSDIQMTQSPSSLSAS |
| | | VGDRVTITCRASQDVNTAVAWYQQKPGKAPKLLIYSASFLYSGVPSRFSGSRSG |
| | | TDFTLTISSLQPEDFATYYCQQHYTTPPTFGQGTKVEIK (SEQ ID NO: |
| | | 232) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.05scFv | | GGRTYYRDSVKGRFTISRDNSKNTLYLQMNSLRAEDTAVYYCAKFRQYSGGFDY |
| | | WGQGTLVTVSSASTKGPSVEPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGAEVKKPGASVKISCKTSGYTFTEYTIHWVKQAS |
| | | GKGLEWIGNINPNNGGTTYNQKFEDRATLTVDKSTSTAYMELSSLRSEDTAVYY |
| | | CAAGWNFDYWGQGTTVTVSSGGGGSGGGGSGGGGSDIVMTQSPSSLSASVGDRV |
| | | TITCKASQDVGTAVDWYQQKPGKAPKLLIYWASTRHTGVPDRFTGSGSGTDFTL |
| | | TISSLQPEDFADYFCQQYNSYPLTFGGGTKLEIK (SEQ ID NO: 233) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.06scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLVQSGSELKKPGASVKVSCKASGYTFTEYGMNVWRQAP |
| | | GQGLEWMGWINTKSGEATYVEEFKGRFVFSLDTSVSTAYLQISSLKAEDTAVYY |
| | | CARWDFYDYVDEAMYWGQGTTVTVSSGGGGSGGGGSGGGGSDIQMTQSPSSLSA |
| | | SVGDRVTITCKASQTVSANVAWYQQKPGKAPKLLIYLASYRYRGVPSRFSGSGS |
| | | GTDFTLTISSLQPEDFATYYCHQYYTYPLFTFGQGTKLEIK (SEQ ID NO: |
| | | 234) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.07scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGPGLVQPGGSVRISCAASGYTFTNYGMNWVKQAP |
| | | GKGLEWMGWINTYTGESTYADSFKGRFTFSLDTSASAAYLQINSLRAEDTAVYY |
| | | CARFAIKGDYWGQGTLLTVSSGGGGGGGGSGGGGSDIQMTQSPSSLSASVGDR |
| | | VTITCRSTKSLLHSNGITYLYWYQQKPGKAPKLLIYQMSNLASGVPSRFSSSGS |
| | | GTDFTLTISSLQPEDFATYYCAQNLEIPRTFGQGTKVELK (SEQ ID NO: |
| | | 235) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.08scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLVQSGAEVKKPGASVKVSCKTSRYTFTEYTIHWVRQAP |
| | | GQRLEWIGGINPNNGIPNYNQKFKGRVTITVDTSASTAYMELSSLRSEDTAVYY |
| | | CARRRIAYGYDEGHAMDYWGQGTLVTVSSGGGGSGGGGSGGGGSDIVMTQSPDS |
| | | LAVSLGERATINCKSSQSLLYSRNQKNYLAWYQQKPGQPPKLLIFWASTRESGV |
| | | PDRFSGSGFGTDFTLTISSLQAEDVAVYYCQQYFSYPLTFGQGTKVEIK (SEQ |
| | | ID NO: 236) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.09scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGGSLRLSCAASGFTFSDSWIHWVRQAP |
| | | GKGLEWVAWISPYGGSTYYADSVKGRFTISADTSKNTAYLQMNSLRAEDTAVYY |
| | | CARRHWPGGFDYWGQGTLVTVSSGGGGSGGGGSGGGGSDIQMTQSPSSLSASVG |
| | | DRVTITCRASQDVSTAVAWYQQKPGKAPKLLIYSASFLYSGVPSRFSGSGSGTD |
| | | FTLTISSLQPEDFATYYCQQYLYHPATFGQGTKVEIK (SEQ ID NO: 237) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.10scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLLESGGGLVQPGGSLRLSCAVSGFTFNSFAMSWVRQAP |
| | | GKGLEWVSAISGSGGGTYYADSVKGRFTISRDNSKNTLYLQMNSLRAEDTAVYF |
| | | CAKDKILWFGEPVFDYWGQGTLVTVSSGGGGSGGGGSGGGGSEIVLTQSPATLS |
| | | LSPGERATLSCRASQSVSSYLAWYQQKPGQAPRLLIYDASNRATGIPARFSGSG |
| | | SGTDFTLTISSLEPEDFAVYYCQQRSNWPPTFGQGTKVEIK (SEQ ID NO: |
| | | 238) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.11scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGAEVKKPGSSVKVSCKASGYTITDSNIHWVRQAP |
| | | GQSLEWIGYIYPYNGGTDYNQKFKNRATLTVDNPTNTAYMELSSLRSEDTAFYY |
| | | CVNGNPWLAYWGQGTLVTVSSGGGGSGGGGSGGGGSDIQLTQSPSTLSASVGDR |
| | | VTITCRASESLDNYGIRFLTWFQQKPGKAPKLLMYAASNQGSGVPSRFSGSGSG |
| | | TEFTLTISSLQPDDFATYYCQQTKEVPWSFGQGTKVEVK (SEQ ID NO: |
| | | 239) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.12scFv | | GGRTYYRDSVKGRFTISRDNSKNTLYLQMNSLRAEDTAVYYCAKFRQYSGGFDY |
| | | WGQGTLVTVSSASTKGPSVEPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGGSLRLSCAASGYTFTSYWLHWVRQAP |
| | | GKGLEWVGMIDPSNSDTRENPNFKDRFTISADTSKNTAYLQMNSLRAEDTAVYY |
| | | CATYRSYVTPLDYWGQGTLVTVSSGGGGSGGGGSGGGGSDIQMTQSPSSLSASV |
| | | GDRVTITCKSSQSLLYTSSQKNYLAWYQQKPGKAPKLLIYWASTRESGVPSRFS |
| | | GSGSGTDFTLTISSLQPEDFATYYCQQYYAYPWTFGQGTKVEIK (SEQ ID |
| | | NO: 240) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.13scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLVQSGAEVKKPGASVKVSCKASGYTFTNYNMHWVRQAP |
| | | GQRLEWMGTIYPGNDDTSYNQKFKDRVTITADTSASTAYMELSSLRSEDTAVYY |
| | | CARGGYRAMDYWGQGTLVTVSSGGGGSGGGGSGGGGSDIVMTQSPLSLPVTPGE |
| | | PASISCRSSQSIVYSNGNTYLGWYLQKPGQSPQLLIYKVSNRFSGVPDRFSGSG |
| | | SGTDFTLKISRVEAEDVGVYYCFQGSHVPYTFGQGTKLEIK (SEQ ID NO: |
| | | 241) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.14scFv | | GGRTYYRDSVKGRFTISRDNSKNTLYLQMNSLRAEDTAVYYCAKFRQYSGGFDY |
| | | WGQGTLVTVSSASTKGPSVEPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQESGPGLVKPSQTLSLTCTVSGGSISSGDYFWSWIRQ |
| | | LPGKGLEWIGHIHNSGTTYYNPSLKSRVTISVDTSKKQFSLRLSSVTAADTAVY |
| | | YCARDRGGDYYYGMDVWGQGTTVTVSSGGGGSGGGGSGGGGSEIVLTQSPGTLS |
| | | LSPGERATLSCRASQGISRSYLAWYQQKPGQAPSLLIYGASSRATGIPDRFSGS |
| | | GSGTDFTLTISRLEPEDFAVYYCQQFGSSPWTFGQGTKVEIK (SEQ ID NO: |
| | | 242) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.15scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQESGPGLVKPSETLSLTCTVSGGSVSSGSYYWSWIRQ |
| | | PPGKGLEWIGYIYYSGSTNYNPSLKSRVTISVDTSKNQFSLKLSSVTAADTAVY |
| | | YCAREGKNGAFDIWGQGTMVTVSSGGGGSGGGGSGGGGSDIQMTQSPSSLSASV |
| | | GDRVTITCRASQSISSYLNWYQQKPGKAPKLLIYAASSLQSGVPSGFSGSGSGT |
| | | DFTLTISSLQPEDFATYYCQQSYSTPLTFGGGTKVEIK (SEQ ID NO: |
| | | 243) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.16scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLLQSGPELEKPGASVMISCKASGSSFTGYNMNWVRQNI |
| | | GKSLEWIGAIDPYYGGTSYNQKFKGRATLTVDKSSSTAYMHLKSLTSEDSAVYY |
| | | CVSGMEYWGQGTSVTVSSGGGGSGGGGSGGGGSEIVMTQSPATLSVSPGERATL |
| | | SCRSSQSLVHRNGNTYLHWYLQKPGQSPKLLIHKVSNRFSGVPDRFSGSGSGTD |
| | | FTLKISRVEAEDLGVYFCSQSTHVPPLTFGAGTKLELK (SEQ ID NO: |
| | | 244) |
| | 2^{nd} chain | CTA.03 VL-Cλ (SEQ ID NO: 24) |
| CTA.03-01Fab/ | 1^{st} chain | EVQLLESGGGLVQPGGSLRLSCAASGFTFSSFPMAWVRQAPGKGLEWVSTISTS |
| CTAT.02- | | GGRTYYRDSVKGRFTISRDNSKNTLYLQMNSLRAEDTAVYYCAKFRQYSGGFDY |
| 01scFv | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDATVKLLIYHTSRLHSGVPSRFSGSGSG |
| | | TDYSLTISNLEQEDIATYFCQQGNTLPYTFGGGTKLEIT (SEQ ID NO: |
| | | 245) |
| | 2^{nd} chain | CTA.03 VL-Cλ-01 (SEQ ID NO: 228) |
| CTA.03-01Fab/ | 1^{st} chain | EVQLLESGGGLVQPGGSLRLSCAASGFTFSSFPMAWVRQAPGKGLEWVSTISTS |
| CTAT.02- | | GGRTYYRDSVKGRFTISRDNSKNTLYLQMNSLRAEDTAVYYCAKFRQYSGGFDY |
| 02scFv | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPEGTVKLLIYHTSRLHSGVPSRFSGSGSG |
| | | TDYSLTISNLEQEDIATYFCQQGNTLPYTFGGGTKLEIT (SEQ ID NO: |
| | | 246) |
| | 2^{nd} chain | CTA.03 VL-Cλ-01 (SEQ ID NO: 228) |
| CTA.03-02Fab/ | 1^{st} chain | EVQLLESGGGLVQPGGSLRLSCAASGFTFSSFPMAWVRQAPGKGLEWVSTISTS |
| CTAT.02- | | GGRTYYRDSVKGRFTISRDNSKNTLYLQMNSLRAEDTAVYYCAKFRQYSGGFDY |
| 02scFv | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPEGTVKLLIYHTSRLHSGVPSRFSGSGSG |
| | | TDYSLTISNLEQEDIATYFCQQGNTLPYTFGGGTKLEIT (SEQ ID NO: |
| | | 247) |
| | 2^{nd} chain | CTA.03 VL-Cλ-02 (SEQ ID NO: 25) |
| CTA.03-02Fab/ | 1^{st} chain | EVQLLESGGGLVQPGGSLRLSCAASGFTFSSFPMAWVRQAPGKGLEWVSTISTS |
| CTAT.02- | | GGRTYYRDSVKGRFTISRDNSKNTLYLQMNSLRAEDTAVYYCAKFRQYSGGFDY |
| 01scFv | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDATVKLLIYHTSRLHSGVPSRFSGSGSG |
| | | TDYSLTISNLEQEDIATYFCQQGNTLPYTFGGGTKLEIT (SEQ ID NO: |
| | | 248) |
| | 2^{nd} chain | CTA.03 VL-Cλ-01 (SEQ ID NO: 25) |
| CTA.03Fab/ | 1^{st} chain | EVQLLESGGGLVQPGGSLRLSCAASGFTFSSFPMAWVRQAPGKGLEWVSTISTS |
| CTAT.17scFv | | GGRTYYRDSVKGRFTISRDNSKNTLYLQMNSLRAEDTAVYYCAKFRQYSGGFDY |
| | | WGQGTLVTVSSASTKGPSVEPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGQVQLQESGPGLVKPSETLSLTCTVSGGSVSSGDYYWTWIRQ |
| | | SPGKGLEWIGHIYYSGNTNYNPSLKSRLTISIDTSKTQFSLKLSSVTAADTAIY |
| | | YCVRDRVTGAFDIWGQGTMVTVSSGGGGSGGGGSGGGGSDIQMTQSPSSLSASV |
| | | GDRVTITCQASQDISNYLNWYQQKPGKAPKLLIYDASNLETGVPSRFSGSGSGT |
| | | DFTFTISSLQPEDIATYFCQHFDHLPLAFGGGTKVEIKR (SEQ ID NO: |
| | | 307) |
| | 2^{nd} chain | CTA.03 VL-Cλ (SEQ ID NO: 24) |
| CTA.03Fab/ | 1^{st} chain | EVQLLESGGGLVQPGGSLRLSCAASGFTFSSFPMAWVRQAPGKGLEWVSTISTS |
| CTAT.19scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVESGGGLVQPGRSLRLSCAASGFTFNDYAMHWVRQAP |
| | | GKGLEWVSTISWNSGSIGYADSVKGRFTISRDNAKKSLYLQMNSLRAEDTALYY |
| | | CAKDIQYGNYYYGMDVWGQGTTVTVSSGGGGSGGGGSGGGGSEIVLTQSPATLS |
| | | LSPGERATLSCRASQSVSSYLAWYQQKPGQAPRLLIYDASNRATGIPARFSGSG |
| | | SGTDFTLTISSLEPEDFAVYYCQQRSNWPITFGQGTRLEIKR (SEQ ID NO: |
| | | 308) |
| | 2^{nd} chain | CTA.03 VL-Cλ (SEQ ID NO: 24) |
| CTA.02Fab/ | 1^{st} chain | QVQLQQSGAELARPGASVKMSCKASGYTFTRYTMHWVKQRPGQGLEWIGYINPS |
| CTAT.02scFv | | RGYTNYNQKFKDKATLTTDKSSSTAYMQLSSLTSEDSAVYYCARYYDDHYCLDY |
| | | WGQGTTLTVSSAKTTAPSVYPLAPVCGGTTGSSVTLGCLVKGYFPEPVTLTWNS |
| | | GSLSSGVHTFPAVLQSDLYTLSSSVTVISSTWPSQSITCNVAHPASSTKVDKKI |
| | | GGGSGGGEVKLQESGPGLVAPSQSLSVTCTVSGVSLPDYGVSWIRQPPRKGLEW |
| | | LGVIWGSETTYYNSALKSRLTIIKDNSKSQVFLKMNSLQTDDTAIYYCAKHYYY |
| | | GGSYAMDYWGQGTSVTVSSGGGGSGGGGSGGGGSDIQMTQTTSSLSASLGDRVT |
| | | ISCRASQDISKYLNWYQQKPDGTVKLLIYHTSRLHSGVPSRFSGSGSGTDYSLT |
| | | ISNLEQEDIATYFCQQGNTLPYTFGGGTKLEIT (SEQ ID NO: 309) |
| | 2^{nd} chain | CTA.02 VL-Cλ (SEQ ID NO: 11) |
| CTA.04Fab/ | 1^{st} chain | QVQLVQSGGGVVQPGRSLRLSCKASGYTFTRYTMHWVRQAPGKGLEWIGYINPS |
| CTAT.02ScFv | | RGYTNYNQKVKDRFTISRDNSKNTAFLQMDSLRPEDIGVYFCARYYDDHYCLDY |
| | | WGQGTPVTVSSASTKGPSVEPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVKLQESGPGLVAPSQSLSVTCTVSGVSLPDYGVSWIRQPP |
| | | RKGLEWLGVIWGSETTYYNSALKSRLTIIKDNSKSQVFLKMNSLQTDDTAIYYC |
| | | AKHYYYGGSYAMDYWGQGTSVTVSSGGGGSGGGGSGGGGSDIQMTQTTSSLSAS |
| | | LGDRVTISCRASQDISKYLNWYQQKPDGTVKLLIYHTSRLHSGVPSRFSGSGSG |
| | | TDYSLTISNLEQEDIATYFCQQGNTLPYTFGGGTKLEIT (SEQ ID NO: |
| | | 310) |
| | 2^{nd} chain | CTA.04 VL-Cλ (SEQ ID NO: 36) |
| CTA.05Fab/ | 1^{st} chain | QVQLVQSGAEVKKPGASVKVSCKASGYTFISYTMHWVRQAPGQGLEWMGYINPR |
| CTAT.02scFv | | SGYTHYNQKLKDKATLTADKSASTAYMELSSLRSEDTAVYYCARSAYYDYDGFA |
| | | YWGQGTLVTVSSASTKGPSVFPLAPCSRSTSESTAALGCLVKDYFPEPVTVSWN |
| | | SGALTSGVHTFPAVLQSSGLYSLSSVAIVTVPSSNFGTQTYTCNVDHKPSNTKV |
| | | DKTVGGGSGGGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAK |
| | | HYYYGGSYAMDYWGQGTSVTVSSGGGGSGGGGSGGGGSDIQMTQTTSSLSASLG |
| | | DRVTISCRASQDISKYLNWYQQKPDGTVKLLIYHTSRLHSGVPSRFSGSGSGTD |
| | | YSLTISNLEQEDIATYFCQQGNTLPYTFGGGTKLEIT (SEQ ID NO: 311) |
| | 2^{nd} chain | CTA.05 VL-Cλ (SEQ ID NO: 47) |
| CTA.02scFv/ | 1^{st} chain | QVQLKQSGPGLVQPSQSLSITCTVSGFSLTNYGVHWVRQSPGKGLEWLGVIWSG |
| CTAT.03Fab | | GNTDYNTPFTSRLSINKDNSKSQVFFKMNSLQSNDTAIYYCARALTYYDYEFAY |
| | | WGQGTLVTVSAASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKR |
| | | VEPKSCDKGGGSGGGQVQLQQSGAELARPGASVKMSCKASGYTFTRYTMHWVKQ |
| | | RPGQGLEWIGYINPSRGYTNYNQKFKDKATLTTDKSSSTAYMQLSSLTSEDSAV |
| | | YYCARYYDDHYCLDYWGQGTTLTVSSGGGGSGGGGSGGGGSQIVLTQSPAIMSA |
| | | SPGEKVTMTCSASSSVSYMNWYQQKSGTSPKRWIYDTSKLASGVPAHFRGSGSG |
| | | TSYSLTISGMEAEDAATYYCQQWSSNPFTFGSGTKLEINR (SEQ ID NO: |
| | | 312) |
| | 2^{nd} chain | CTAT.03 VL-Cλ (SEQ ID NO: 84) |
| CTA.03scFv/ | 1^{st} chain | QVQLKQSGPGLVQPSQSLSITCTVSGFSLTNYGVHWVRQSPGKGLEWLGVIWSG |
| CTAT.03Fab | | GNTDYNTPFTSRLSINKDNSKSQVFFKMNSLQSNDTAIYYCARALTYYDYEFAY |
| | | WGQGTLVTVSAASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKR |
| | | VEPKSCDKGGGSGGGEVQLLESGGGLVQPGGSLRLSCAASGFTFSSFPMAWVRQ |
| | | APGKGLEWVSTISTSGGRTYYRDSVKGRFTISRDNSKNTLYLQMNSLRAEDTAV |
| | | YYCAKFRQYSGGFDYWGQGTLVTVSSGGGGSGGGGSGGGGSDIQLTQPNSVSTS |
| | | LGSTVKLSCTLSSGNIENNYVHWYQLYEGRSPTTMIYDDDKRPDGVPDRFSGSI |
| | | DRSSNSAFLTIHNVAIEDEAIYFCHSYVSSFNVFGGGTKLTVLRQ (SEQ ID |
| | | NO: 313) |
| | 2^{nd} chain | CTAT.03 VL-Cλ (SEQ ID NO: 84) |
| CTA.04scFv/ | 1^{st} chain | QVQLKQSGPGLVQPSQSLSITCTVSGFSLTNYGVHWVRQSPGKGLEWLGVIWSG |
| CTAT.03Fab | | GNTDYNTPFTSRLSINKDNSKSQVFFKMNSLQSNDTAIYYCARALTYYDYEFAY |
| | | WGQGTLVTVSAASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKR |
| | | VEPKSCDKGGGSGGGQVQLVQSGGGVVQPGRSLRLSCKASGYTFTRYTMHWVRQ |
| | | APGKGLEWIGYINPSRGYTNYNQKVKDRFTISRDNSKNTAFLQMDSLRPEDTGV |
| | | YFCARYYDDHYCLDYWGQGTPVTVSSGGGGSGGGGSGGGGSDIQMTQSPSSLSA |
| | | SVGDRVTITCSASSSVSYMNWYQQTPGKAPKRWIYDTSKLASGVPSRFSGSGSG |
| | | TDYTFTISSLQPEDIATYYCQQWSSNPFTFGQGTKLQITR (SEQ ID NO: |
| | | 314) |
| | 2^{nd} chain | CTAT.03 VL-Cλ (SEQ ID NO: 84) |
| CTA.05scFv/ | 1^{st} chain | QVQLKQSGPGLVQPSQSLSITCTVSGFSLTNYGVHWVRQSPGKGLEWLGVIWSG |
| CTAT.03Fab | | GNTDYNTPFTSRLSINKDNSKSQVFFKMNSLQSNDTAIYYCARALTYYDYEFAY |
| | | WGQGTLVTVSAASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKR |
| | | VEPKSCDKGGGSGGGEVQLVQSGAEVKKPGASVKISCKTSGYTFTEYTIHWVKQ |
| | | ASGKGLEWIGNINPNNGGTTYNQKFEDRATLTVDKSTSTAYMELSSLRSEDTAV |
| | | YYCAAGWNFDYWGQGTTVTVSSGGGGSGGGGSGGGGSDIVMTQSPSSLSASVGD |
| | | RVTITCKASQDVGTAVDWYQQKPGKAPKLLIYWASTRHTGVPDRFTGSGSGTDF |
| | | TLTISSLQPEDFADYFCQQYNSYPLTFGGGTKLEIK (SEQ ID NO: 315) |
| | 2^{nd} chain | CTAT.03 VL-Cλ (SEQ ID NO: 84) |
| CTA03-02Fab/ | 1^{st} chain | EVQLLESGGGLVQPGGSLRLSCAASGFTFSSFPMAWVRQAPGKGLEWVSTISTS |
| CTAT.05scFv | | GGRTYYRDSVKGRFTISRDNSKNTLYLQMNSLRAEDTAVYYCAKFRQYSGGFDY |
| | | WGQGTLVTVSSASTKGPSVFPLAPSSKSTSGGTAALGCLVKDYFPEPVTVSWNS |
| | | GALTSGVHTFPAVLQSSGLYSLSSVVTVPSSSLGTQTYICNVNHKPSNTKVDKK |
| | | VEPKSCGGGSGGGEVQLVQSGAEVKKPGASVKISCKTSGYTFTEYTIHWVKQAS |
| | | GKGLEWIGNINPNNGGTTYNQKFEDRATLTVDKSTSTAYMELSSLRSEDTAVYY |
| | | CAAGWNFDYWGQGTTVTVSSGGGGSGGGGSGGGGSDIVMTQSPSSLSASVGDRV |
| | | TITCKASQDVGTAVDWYQQKPGKAPKLLIYWASTRHTGVPDRFTGSGSGTDFTL |
| | | TISSLQPEDFADYFCQQYNSYPLTFGGGTKLEIK (SEQ ID NO: 316) |
| | 2^{nd} chain | CTA.03 VL-Cλ-02 (SEQ ID NO: 25) |

### B. Methods for Producing Bispecific Antibodies

Any of the bispecific antibodies described herein can be made by any method known in the art. See, for example, Harlow and Lane, (1998) Antibodies: A Laboratory Manual, Cold Spring Harbor Laboratory, New York. In some embodiments, the anti-CD3 antibody and/or the anti-TAA antibody for use in making the bispecific antibodies may be produced by the conventional hybridoma technology. Alternatively, the anti-CD3 and/or anti-TAA antibody may be identified from a suitable library (e.g., a human antibody library). In some instances, high affinity fully human CD3 and/or TAA binders may be obtained from a human antibody library, for example, affinity maturation libraries (e.g., having variations in one or more of the CDR regions). There are a number of routine methods known in the art to identify and isolate antibodies capable of binding to the target antigens described herein, including phage display, yeast display, ribosomal display, or mammalian display technology.

In some embodiments, the bispecific antibodies disclosed herein may be produced by the conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide.

In some instances, nucleic acids encoding the one or both chains of a bispecific antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both chains of a bispecific antibody as described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, each encoding one chain of a bispecific antibody disclosed herein. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the bispecific antibody as described herein, vectors (e.g., expression vectors) containing such; and host cells comprising the vectors are within the scope of the present disclosure. Methods for producing such bispecific antibodies (e.g., using host cells via the recombinant technology) are also within the scope of the present disclosure.

III. Cancer Treatment with Armed Immune Cells

The armed immune cells produced by any of the methods disclosed herein, which are also within the scope of the present disclosure, may be used in cancer treatment. Accordingly, also provided herein is a method for treating cancer using the armed immune cells disclosed herein. To practice the method disclosed herein, an effective amount of the armed immune cells or a pharmaceutical composition comprising such can be administered to a subject (e.g., a human) in need of the treatment via a suitable route, such as intravenous administration, e.g., as a bolus or by continuous infusion over a period of time. In some instances, the armed immune cells are autologous to the subject. In other instances, the armed immune cells are allogenic to the subject.

The subject to be treated by the methods described herein can be a mammal, more preferably a human or a non-human primate. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying tumor cells expressing the target TAA, to which a bispecific antibody binds. Exemplary cancers include, but are not limited to, melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, and myeloma.

Presence of specific tumor associated antigens by specific types of cancer cells are known in the art. For example, B-cell malignancies often involve CD19+(e.g., B-cell acute lymphoblastic leukemia) and/or CD20+ cancer cells (e.g., B-cell Non-Hodgkin's lymphoma). EGFR is expressed on various types of cancer, such as lung cancer and colon cancer. HER2 is associated with, for example, breast cancer. PSMA is associated, for example, prostate cancer. CEA is associated with various types of cancer, including colon, rectum, and pancreatic cancer. EpCAM, FAP, CD47, and TRAIL-R2 are associated with solid tumors. PDL1 is associated with various cancers, such as bladder cancer, non-small cell lung cancer, breast cancer, small cell lung cancer, etc. CD38 is associated with, for example, multiple myeloma. CD33 is associated with, for example, AML. cMET (HGFR) is associated, for example, non-small cell lung cancer. Mesothelin is associated with mesothelioma. GD2 is associated with neuroblastoma. Accordingly, choosing a bispecific antibody disclosed herein that has a suitable anti-TAA binding moiety to treat a particular type of cancer is within the knowledge of a medical practitioner.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery. A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of armed immune cells as described herein will depend on the specific bispecific antibody on the immune cells, the type of immune cells (or compositions thereof) employed, the type and severity of the disease/disorder, the patient's clinical history and response to the agonist, and the discretion of the attending physician. Typically the clinician will administer armed immune cells, until a dosage is reached that achieves the desired result. Methods of determining whether a dosage resulted in the desired result would be evident to one of skill in the art. Administration of one or more doses of armed immune cells can be continuous or intermittent, depending, for example, upon the recipient's physiological condition, whether the purpose of the administration is therapeutic or prophylactic, and other factors known to skilled practitioners. The administration of the armed immune cells may be essentially continuous over a preselected period of time or may be in a series of spaced dose, e.g., either before, during, or after developing a target disease or disorder.

In some embodiments, the amount of the armed immune cells such as armed T cells administered to the subject can be about 1×10⁴ to 1×10⁷ cells/kg body weight of the subject. In certain embodiments, the amount of armed immune cells such as armed T cells can be administered to the subject from about 1×10⁵ to 1×10⁶ cells/kg body weight of the subject. The dose can be administered in a single dose, or alternatively in more than one dose.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the treatment method as disclosed herein may be performed to a human cancer patient having a target cancer. Exemplary cancers include, but are not limited to, human cancer patient has melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, or myeloma.

Conventional methods, known to those of ordinary skill in the art of medicine, can be used to administer the armed immune cells or a pharmaceutical composition comprising such to a subject, depending upon the type of cancer to be treated or the site of the cancer. In some instances, the armed immune cells can be administered via intravenous infusion.

In some embodiments, the armed immune cells disclosed herein may be co-used with another anti-cancer agent, for example, a chemotherapeutic agent, an immunotherapeutic agent, or a combination thereof. For example, the armed immune cells disclosed herein may be used in combination with an immune checkpoint inhibitor, such as an anti-PD-1 antibody or an anti-PDL1 antibody. As used herein, the term “combination,” “combined,” and related terms refers to the simultaneous or sequential administration of multiple therapeutic agents in accordance with this disclosure. For example, the armed immune cells as disclosed herein may be administered with another therapeutic agent simultaneously or sequentially in separate unit dosage forms or together in a single unit dosage form.

In some embodiments, the armed immune cells can be prepared at a point of care by a method disclosed herein and be given to a patient onsite. In some examples, the armed immune cells may be autologous to the patient. Alternatively, the armed immune cells may be allogenic to the patient. A point of care as disclosed herein refers to a clinical site (e.g., a hospital, a clinic, or a doctor's office) where cell therapy can be performed. In some examples, blood samples may be collected from a human patient (e.g., a human cancer patient) and immune cells therein may be isolated following the guidance provided herein. The isolated immune cells can then be armed with a suitable BsAb as also disclosed herein and the resultant armed immune cells can be administered to the same human patient onsite.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Preparation of Armed T Cells

This example illustrates a rapid in vitro preparation process for producing T cells armed with an exemplary bispecific antibody capable of binding to both CD3 and EGFR. An exemplary preparation process is illustrated in FIG. 1.

(a) Isolation of T Cells from Peripheral Blood Mononuclear Cells (PBMCs)

Blood samples were collected from a healthy human donor and the peripheral blood mononuclear cells (PBMCs) were isolated by conventional technology. Briefly, the whole blood from the healthy donor was diluted with PBS, which was then gently layered over an equal volume of Ficoll® in a Falcon® tube and centrifuged for 30-40 minutes at 400-500 g. The layer containing PBMCs was collected.

CD3⁺ T cells or subtypes thereof (e.g., CD8⁺ T cells, CD4⁺ T cells, or Treg cells) were then isolated from the PBMCs via negative- or positive-selection. Examples of T cell isolation kits listed below:

### Negative Selection Kit:

CD8 T cell isolation: MagniSort™ Human CD8 Naïve T cell Enrichment Kit, Dynabeads™ Untouched™ Human CD8 T Cells Kit, MojoSort™ Human CD8 T Cell Isolation Kit.
CD4 T cell isolation: MagniSort™ Human CD4 Naïve T cell Enrichment Kit, Dynabeads™ Untouched™ Human CD4 T Cells Kit, CD4+ T Cell Isolation Kit (negative selection).
Treg isolation: EasySep™ Human CD4+CD127lowCD49d− Regulatory T Cell Enrichment Kit.
   Positive selection kit:
CD8 T cell isolation: MagniSort™ Human CD8 Positive Selection Kit, EasySep™ Human CD8 Positive Selection Kit, MojoSort Human CD8 Nanobeads.
CD4 T cell isolation: EasySep™ Release Human CD4 Positive Selection Kit, Dynabeads™ CD4 Positive Isolation Kit, MojoSort Human CD4 Nanobeads.
Treg isolation: EasySep™ Human CD25 Positive Selection kit, Dynabeads™ Regulatory CD4+/CD25+ T Cell Kit, MagCellect Human CD4+CD25+ Regulatory T Cell Isolation Kit.

The immune cell populations before or after T cell isolation were analyzed by flow cytometry. Briefly, the cells were stained with APC-conjugated anti-CD3 antibody and PE-conjugated anti-CD56 antibody. The fluorescent signal of these cells was analyzed by flow cytometer. The results indicate that the PBMCs before T cell isolation contain about 56.1% CD3+ T cells, while the T cell percentage increased to about 97.43% in the isolated T cell population. This indicates that the purification process provided herein led to production of CD3+ T cells with high purity.

### (b) Preparation of Bi-Specific Antibody Armed T Cells

CD3⁺ T cells isolated as described above were incubated with exemplary_anti-CD3/anti-EGFR BsAbs CTA02/CTAT03 or CTA03/CTAT03, at about 37° C. for about 1 hours to form armed T cells. After the incubations, the T cells were analyzed by flow cytometer to measure the level of armed T cell formation. Briefly, the cells were stained with APC-conjugated anti-CD3 antibody and FITC-conjugated anti-Human IgG Fab to identified T cells displaying the bispecific antibodies on the surface. The fluorescent signal was analyzed by flow cytometer. The results are shown in Table 4 below.

TABLE 4
Level of Armed T cells

| Group | % of Armed T cells |
|---|---|
| CTA02/CTAT03 | 99.98% |
| CTA03/CTAT03 | 99.76% |

The results indicate that the process disclosed herein led to formation of armed T cells at a high level (>99%).

### Example 2: Anti-Tumor Activity of Bispecific Antibody (BsAb)-Armed T Cells

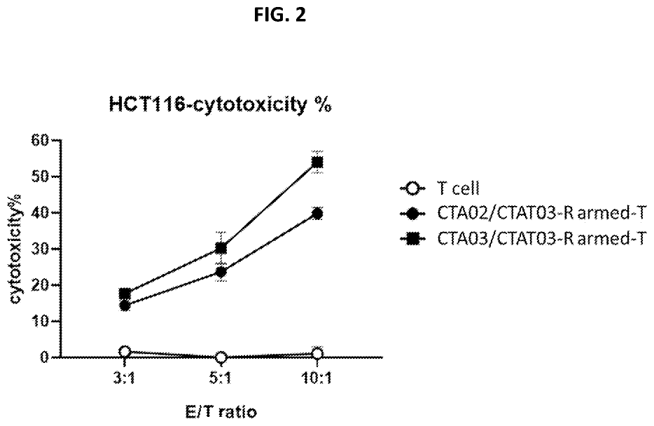

The BsAb-armed T cells produced as described in Example 1 above were investigated for their anti-tumor activity. CTA02/CTAT03-R armed and CTA03/CTAT03-R armed T cells were co-cultured with EGFR*colorectal carcinoma (HCT-116) at different effector cell: target cell ratios (3:1, 5:1, and 10:1) for 18 hr. Tumor cell cytotoxicity was determined with CytoTox 96® Non-Radioactive Cytotoxicity Assay (Promega, G1780). As shown in FIG. 2, T cells isolated from PBMCs had no ability to kill HCT-116, but the BsAb-Armed T cells (both CTA02/CTAT03-R armed-T and CTA03/CTAT03-R armed-T) effectively killed HCT-116 at all tested E:T ratios. Data represent mean±SD.

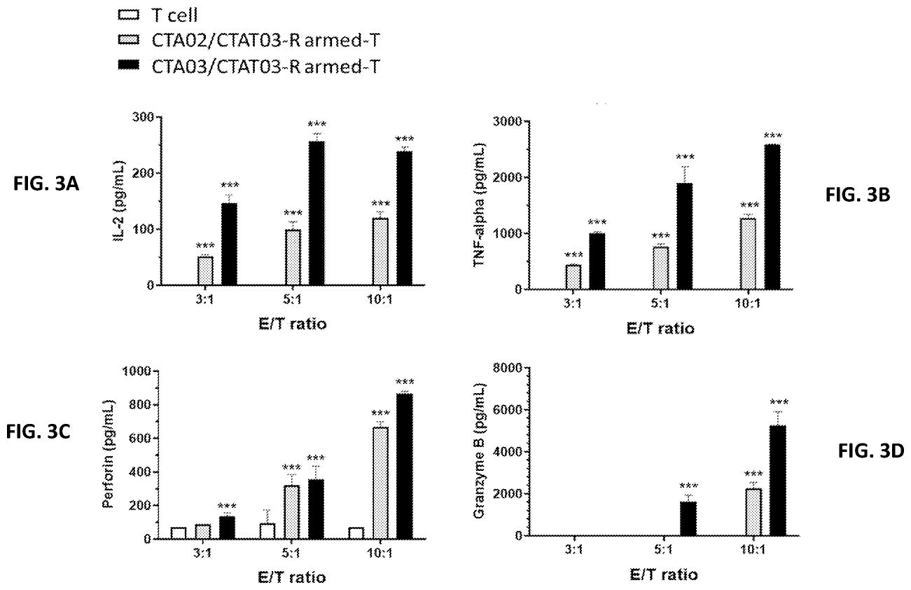

Further, supernatant samples of the co-culture were collected and analyzed for cytokine levels, including human IL-2, TNF-α, human perforin, and human granzyme B. T cells isolated from PBMCs showed a very low level of perforin secretion and no detectable secretion of IL-2, TNF-α, and Granzyme B at all tested E:T ratios. Differently, the BsAb armed T cells showed high levels of cytokine secretion. FIGS. 3A-3D.

In sum, the results of this study show anti-tumor activities of the tested BsAb-armed T cells.

### Example 3: Cytotoxicity of T Cells Armed with Bispecific Antibody (BsAb) Against Target Cancer Cells

Cytotoxicity of armed T cells prepared by the process described in Example 1 above was examined following the assay method provided in Example 2 above. In this study, the ratio of the armed T cells to target cancer cells is at 5:1.

(i) Bispecific Antibodies Comprising CTA.03Fab in Pair with Different Tumor Antigen-Binding Moieties

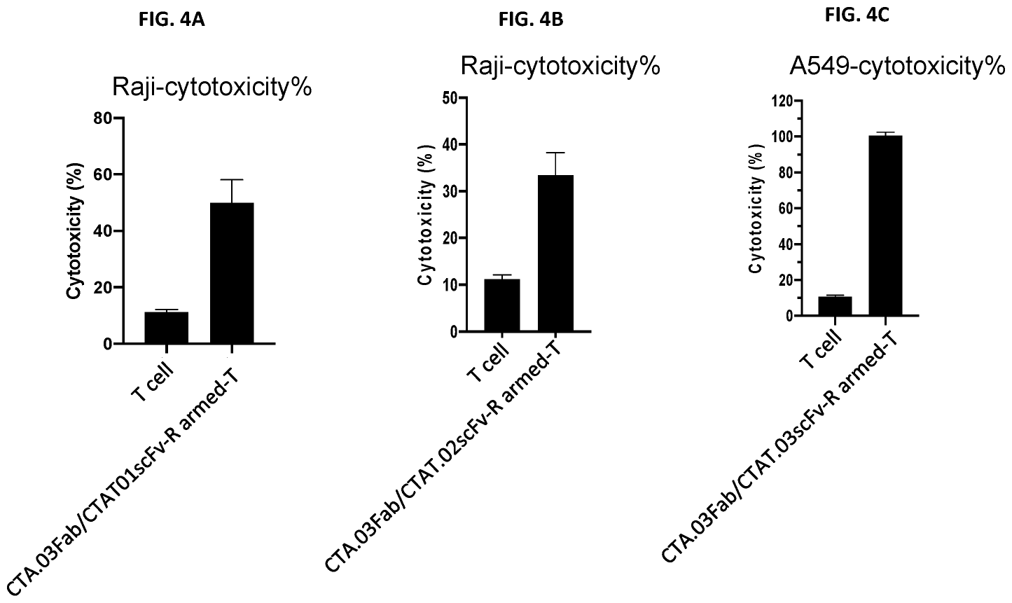

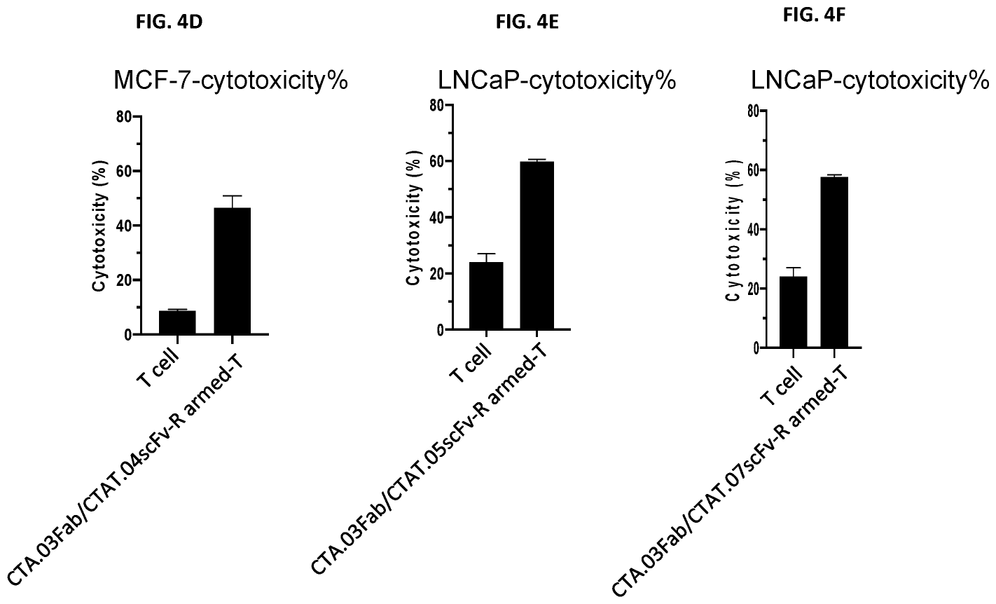

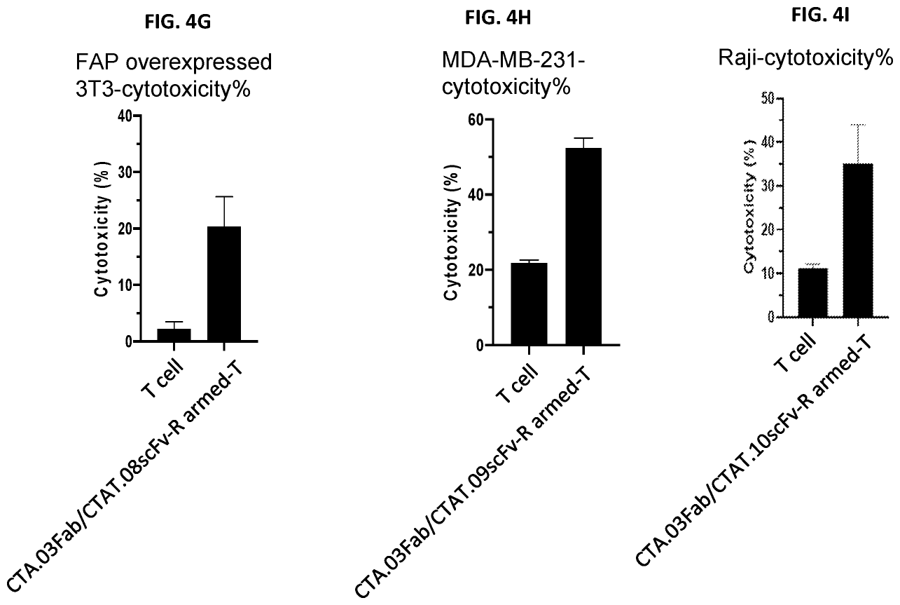

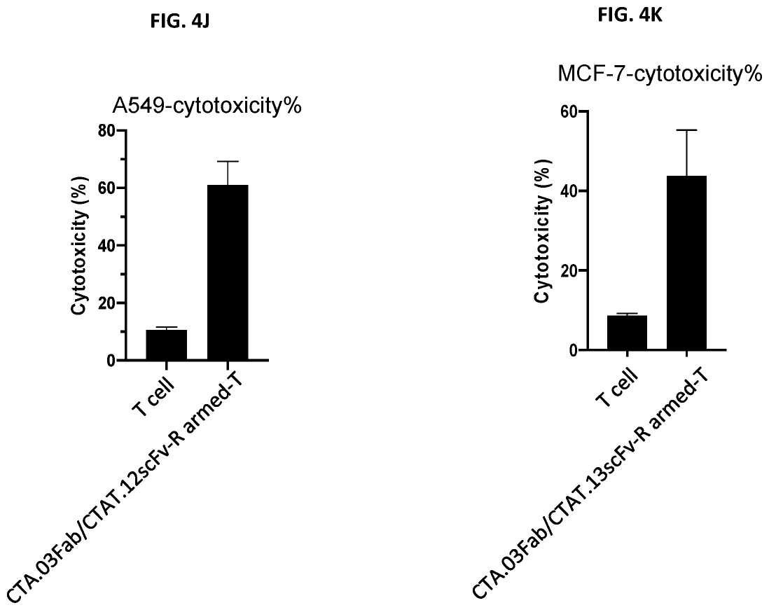

To investigate whether different tumor antigens would affect cytotoxicity of armed T cells prepared by the methods disclosed herein, T cells armed with bispecific antibodies comprising the anti-CD3 moiety CTA.03Fab and an anti-tumor antigen moiety in scFv format were examined for their cytotoxicity against target cancer cells that express the tumor antigen. As shown in FIGS. 4A-4K, all tested armed T cells showed high cytotoxicity against the target cancer cells in vitro. These results indicate that the armed T cells, prepared by the rapid methods disclosed herein, can be used to target various cancer cells expressing different tumor antigens.

(ii) Bispecific Antibodies Comprising Same Anti-CD3 Moiety in Pair with Different Binding Moieties Specific to the Same Tumor Antigen

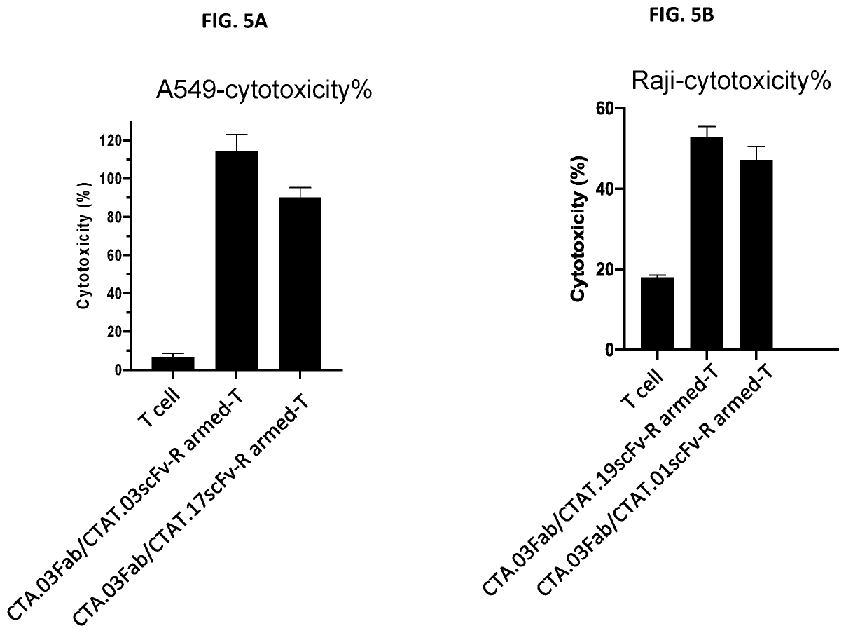

To investigate whether different binding moieties to the same tumor antigen could affect cytotoxicity of armed T cells prepared by the method disclosed herein, T cells armed with bispecific antibodies comprising the same anti-CD3 moiety (CTA.03Fab as an example) in pair with different binding moieties to EGFR or different binding moieties to CD20 were examined in this study. As shown in FIGS. 5A and 5B, all tested armed T cells exhibited high in vitro cytotoxicity against target cancer cells, indicating that the bispecific antibodies used herein do not require specific binding moieties to a target tumor antigen.

(iii) Bispecific Antibodies Comprising Different Anti-CD3 Moieties in Pair with the Same Anti-Tumor Antigen Moiety

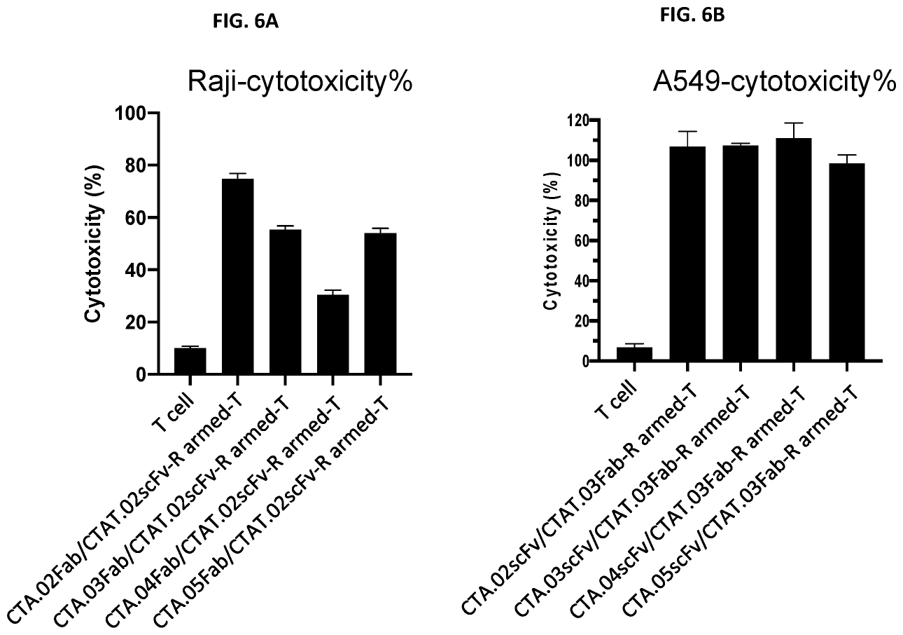

To investigate whether specific anti-CD3 moiety would affect cytotoxicity of bispecific antibodies located on armed T cells, T cells armed with bispecific antibodies comprising different anti-CD3 moieties in combination with the same anti-CD19 moiety (CTAT.02 as an example). As shown in FIG. 6A, the tested armed T cells, comprising bispecific antibodies having different anti-CD3 moieties in Fab format in combination with CTAT.02scFv, exhibited high in vitro cytotoxicity. Similar results were observed when the anti-CD3 moiety is in scFv format and the CTAT.02 moiety is in Fab format. FIG. 6B.

### (iv) Bispecific Antibodies Comprising Mutated Anti-CD3 or Anti-Tumor Antigen Moieties

Point mutations were introduced into CTA.03 to produce mutants CTA.03-02 as shown in Table 1 above. Similarly, point mutations were introduced into CTAT.02 to produce mutants CTAT.02-01 and CTAT.02-02 as shown in Table 2 above.

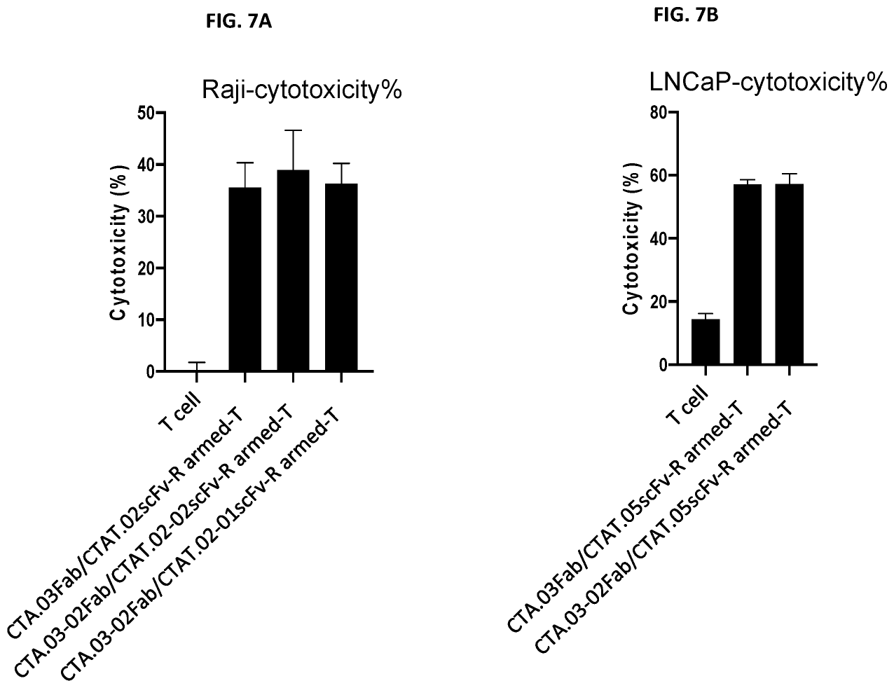

Cytotoxicity of armed T cells comprising bispecific antibodies containing such mutated binding moieties were examined. As shown in FIG. 7A, T cells armed with bispecific antibodies comprising both mutated anti-CD3 and mutated anti-CD19 moieties showed similar cytotoxicity as the wild-type counterpart against Raji cells. Similarly, T cells armed with bispecific antibodies comprising mutated anti-CD3 moiety in combination with CTAT.05 showed similar cytotoxicity relative to the wild-type counterpart against LNCap, a prostate cancer cell line.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. In some embodiments, the hinge domain is a hinge domain of a naturally occurring protein.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for preparing armed immune cells, comprising:
(i) isolating a population of CD3⁺ immune cells from a human blood sample; and
(ii) contacting the population of immune cells with a bi-specific antibody specific to CD3 and a tumor associated antigen (TAA) to produce armed immune cells, which display the bi-specific antibody on the cell surface.

2. The method of claim 1, wherein the population of CD3+ immune cells comprises CD8+ T cells, CD4+ T cells, natural killer (NK) T cells, regulatory T (T_{reg}) cells, or a combination thereof.

3. The method of claim 2, wherein the population of CD3+ immune cells are substantially CD8+ T cells.

4. The method of claim 2, wherein the population of CD3+ immune cells are substantially CD4+ T cells.

5. The method of claim 2, wherein the population of CD3+ immune cells are substantially NK T cells.

6. The method of claim 2, wherein the population of CD3+ immune cells are substantially T_{reg} cells.

7. The method of claim 1, wherein the human blood sample is a peripheral blood mononuclear cell (PBMC) sample.

8. The method of claim 1, wherein the human blood sample is obtained from a human donor.

9. The method of claim 1, wherein the human blood sample is obtained from a human cancer patient.

10. The method of claim 1, wherein step (i) comprises negative selection.

11. The method of claim 1, wherein step (i) comprises positive selection.

12. The method of claim 1, wherein step (ii) comprises incubating the population of immune cells with the bi-specific antibody at a temperature of about 4-37° C. for about 30 minutes to 2 hours.

13. The method of claim 1, wherein step (i) and step (ii) are performed concurrently.

14. The method of claim 1, further comprising administering the armed immune cells to a human patient in need thereof.

15. The method of claim 14, wherein the armed immune cells are autologous to the human patient.

16. The method of claim 14, wherein the armed immune cells are allogenic to the human patient.

17. The method of claim 1, further comprising placing the armed immune cells in a cryopreservation solution for storage.

18. The method of claim 1, wherein the bi-specific antibody comprises a first antigen binding fragment that binds human CD3, wherein the first antigen binding fragment comprises a first heavy chain that comprises a first heavy chain variable region (V_{H}) and a first light chain that comprises a first light chain variable region (V_{L}), wherein the first V_{H} comprises the same heavy chain complementary determining regions (CDRs) or no more than 5 amino acid variations relative to a first reference antibody and the first V_{L} comprises the same light chain CDRs or no more than 5 amino acid variations relative to the reference antibody, and wherein the first reference antibody is CTA.02, CTA.03, CTA.04, or CTA.05.

19. The method of claim 18, wherein the first heavy chain and the first light chain comprise the same V_{H} and V_{L} as the reference antibody.

20. The method of claim 1, wherein the bi-specific antibody comprises a second antigen binding fragment that binds the TAA, which is CD20, CD19, EGFR, HER2, PSMA, CEA, EpCAM, FAP, PD-L1, CD38, CD33, cMET, CD47, TRATL-R2, mesothelin, or GD2.

21. A method for treating cancer, comprising administering to a human cancer patient a population of armed immune cells, which is obtained from a method of claim 1.

22. The method of claim 21, wherein the human cancer patient comprises cancer cells expressing the TAA, to which the bi-specific antibody binds.

23. The method of claim 21, wherein the population of armed immune cells are autologous to the human patient.

24. The method of claim 21, wherein the human cancer patient has melanoma, esophageal carcinoma, gastric carcinoma, brain tumor, small cell lung cancer, non-small cell lung cancer, bladder cancer, breast cancer, pancreatic cancer, colon cancer, rectal cancer, colorectal cancer, renal cancer, hepatocellular carcinoma, ovary cancer, prostate cancer, thyroid cancer, testis cancer, head and neck squamous cell carcinoma, leukemia, lymphoma, or myeloma.


SEQUENCE LISTING (316 sequences)

SEQ ID NO: 1 - protein, 5 aa, synthetic construct
RYTMH

SEQ ID NO: 2 - protein, 17 aa, synthetic construct
YINPSRGYTNYNQKFKD

SEQ ID NO: 3 - protein, 10 aa, synthetic construct
YYDDHYCLDY

SEQ ID NO: 4 - protein, 119 aa, synthetic construct [sequence omitted]

SEQ ID NO: 5 - protein, 10 aa, synthetic construct
SASSSVSYMN

SEQ ID NO: 6 - protein, 7 aa, synthetic construct
DTSKLAS

SEQ ID NO: 7 - protein, 9 aa, synthetic construct
QQWSSNPFT

SEQ ID NO: 8 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 9 - protein, 248 aa, synthetic construct
GGGSGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 10 - protein, 216 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKI

SEQ ID NO: 11 - protein, 214 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECS

SEQ ID NO: 12 - protein, 5 aa, synthetic construct
SFPMA

SEQ ID NO: 13 - protein, 17 aa, synthetic construct
TISTSGGRTYYRDSVKG

SEQ ID NO: 14 - protein, 10 aa, synthetic construct
FRQYSGGFDY

SEQ ID NO: 15 - protein, 119 aa, synthetic construct [sequence omitted]

SEQ ID NO: 16 - protein, 13 aa, synthetic construct
TLSSGNIENNYVH

SEQ ID NO: 17 - protein, 7 aa, synthetic construct
DDDKRPD

SEQ ID NO: 18 - protein, 7 aa, synthetic construct
DDDKRPE

SEQ ID NO: 19 - protein, 9 aa, synthetic construct
HSYVSSFNV

SEQ ID NO: 20 - protein, 112 aa, synthetic construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQ

SEQ ID NO: 21 - protein, 112 aa, synthetic construct
DIQLTQPNSVSTSLGSTVKLSCTLSSGNIENNYVHWYQLYEGRSPTTMIYDDDKRPDAVPDRFSGSIDRSSNSAFLTIHNVAIEDEAIYFCHSYVSSFNVFGGGTKLTVLRQ

SEQ ID NO: 22 - protein, 253 aa, synthetic construct
GGGSGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSGGGGSGGGGSGGGGSDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQ

SEQ ID NO: 23 - protein, 222 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 24 - protein, 216 aa, synthetic construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 216 aa, synthetic construct
DIQLTQPNSVSTSLGSTVKLSCTLSSGNIENNYVHWYQLYEGRSPTTMIYDDDKRPEGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 5 aa, synthetic construct
RYTMH

SEQ ID NO: 27 - protein, 17 aa, synthetic construct
YINPSRGYTNYNQKVKD

SEQ ID NO: 28 - protein, 10 aa, synthetic construct
YYDDHYCLDY

SEQ ID NO: 29 - protein, 119 aa, synthetic construct [sequence omitted]

SEQ ID NO: 30 - protein, 10 aa, synthetic construct
SASSSVSYMN

SEQ ID NO: 31 - protein, 7 aa, synthetic construct
DTSKLAS

SEQ ID NO: 32 - protein, 9 aa, synthetic construct
QQWSSNPFT

SEQ ID NO: 33 - protein, 107 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 34 - protein, 248 aa, synthetic construct
GGGSGGGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 35 - protein, 222 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 5 aa, synthetic construct
SYTMH

SEQ ID NO: 38 - protein, 17 aa, synthetic construct
YINPRSGYTHYNQKLKD

SEQ ID NO: 39 - protein, 11 aa, synthetic construct
SAYYDYDGFAY

SEQ ID NO: 40 - protein, 120 aa, synthetic construct [sequence omitted]

SEQ ID NO: 41 - protein, 10 aa, synthetic construct
SASSSVSYMN

SEQ ID NO: 42 - protein, 7 aa, synthetic construct
DTSKLAS

SEQ ID NO: 43 - protein, 9 aa, synthetic construct
QQWSSNPPT

SEQ ID NO: 44 - protein, 106 aa, synthetic construct [sequence omitted]

SEQ ID NO: 45 - protein, 248 aa, synthetic construct
GGGSGGGQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIK

SEQ ID NO: 46 - protein, 220 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPCSRSTSESTAALGCLVKDYFPEPVTVSWNSGALTSGVHTFPAVLQSSGLYSLSSVAIVTVPSSNFGTQTYTCNVDHKPSNTKVDKTV

SEQ ID NO: 47 - protein, 213 aa, synthetic construct
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 48 - protein, 5 aa, synthetic construct
SYNMH

SEQ ID NO: 49 - protein, 17 aa, synthetic construct
AIYPGNGDTSYNQKFKG

SEQ ID NO: 50 - protein, 12 aa, synthetic construct
STYYGGDWYFNV

SEQ ID NO: 51 - protein, 121 aa, synthetic construct [sequence omitted]

SEQ ID NO: 52 - protein, 10 aa, synthetic construct
RASSSVSYIH

SEQ ID NO: 53 - protein, 7 aa, synthetic construct
ATSNLAS

SEQ ID NO: 54 - protein, 9 aa, synthetic construct
QQWTSNPPT

SEQ ID NO: 55 - protein, 106 aa, synthetic construct [sequence omitted]

SEQ ID NO: 56 - protein, 249 aa, synthetic construct
GGGSGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 57 - protein, 224 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSC

SEQ ID NO: 58 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 5 aa, synthetic construct
DYGVS

SEQ ID NO: 60 - protein, 16 aa, synthetic construct
VIWGSETTYYNSALKS

SEQ ID NO: 61 - protein, 12 aa, synthetic construct
HYYYGGSYAMDY

SEQ ID NO: 62 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 63 - protein, 11 aa, synthetic construct
RASQDISKYLN

SEQ ID NO: 64 - protein, 7 aa, synthetic construct
HTSRLHS

SEQ ID NO: 65 - protein, 9 aa, synthetic construct
QQGNTLPYT

SEQ ID NO: 66 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 67 - protein, 107 aa, synthetic construct
DIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 68 - protein, 107 aa, synthetic construct
DIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 69 - protein, 249 aa, synthetic construct
GGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 70 - protein, 249 aa, synthetic construct
GGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 71 - protein, 249 aa, synthetic construct
GGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 72 - protein, 225 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 73 - protein, 214 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 74 - protein, 5 aa, synthetic construct
NYGVH

SEQ ID NO: 75 - protein, 16 aa, synthetic construct
VIWSGGNTDYNTPFTS

SEQ ID NO: 76 - protein, 11 aa, synthetic construct
ALTYYDYEFAY

SEQ ID NO: 77 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 78 - protein, 11 aa, synthetic construct
RASQSIGTNIH

SEQ ID NO: 79 - protein, 7 aa, synthetic construct
YASESIS

SEQ ID NO: 80 - protein, 9 aa, synthetic construct
QQNNNWPTT

SEQ ID NO: 81 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 82 - protein, 248 aa, synthetic construct
GGGSGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 83 - protein, 224 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 84 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 85 - protein, 5 aa, synthetic construct
DTYIH

SEQ ID NO: 86 - protein, 17 aa, synthetic construct
RIYPTNGYTRYADSVKG

SEQ ID NO: 87 - protein, 11 aa, synthetic construct
WGGDGFYAMDY

SEQ ID NO: 88 - protein, 120 aa, synthetic construct [sequence omitted]

SEQ ID NO: 89 - protein, 11 aa, synthetic construct
RASQDVNTAVA

SEQ ID NO: 90 - protein, 7 aa, synthetic construct
SASFLYS

SEQ ID NO: 91 - protein, 9 aa, synthetic construct
QQHYTTPPT

SEQ ID NO: 92 - protein, 107 aa, synthetic construct [sequence omitted]

SEQ ID NO: 93 - protein, 249 aa, synthetic construct
GGGSGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 94 - protein, 225 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 95 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 96 - protein, 5 aa, synthetic construct
EYTIH

SEQ ID NO: 97 - protein, 17 aa, synthetic construct
NINPNNGGTTYNQKFED

SEQ ID NO: 98 - protein, 6 aa, synthetic construct
GWNFDY

SEQ ID NO: 99 - protein, 115 aa, synthetic construct
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSS

SEQ ID NO: 100 - protein, 11 aa, synthetic construct
KASQDVGTAVD

SEQ ID NO: 101 - protein, 7 aa, synthetic construct
WASTRHT

SEQ ID NO: 102 - protein, 9 aa, synthetic construct
QQYNSYPLT

SEQ ID NO: 103 - protein, 107 aa, synthetic construct
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 104 - protein, 244 aa, synthetic construct
GGGSGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 105 - protein, 220 aa, synthetic construct
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 106 - protein, 214 aa, synthetic construct
DIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 107 - protein, 5 aa, synthetic construct
EYGMN

SEQ ID NO: 108 - protein, 17 aa, synthetic construct
WINTKSGEATYVEEFKG

SEQ ID NO: 109 - protein, 12 aa, synthetic construct
WDFYDYVDEAMY

SEQ ID NO: 110 - protein, 121 aa, synthetic construct
QVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSS

SEQ ID NO: 111 - protein, 11 aa, synthetic construct
KASQTVSANVA

SEQ ID NO: 112 - protein, 7 aa, synthetic construct
LASYRYR

SEQ ID NO: 113 - protein, 10 aa, synthetic construct
HQYYTYPLFT

SEQ ID NO: 114 - protein, 108 aa, synthetic construct
DIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 115 - protein, 251 aa, synthetic construct
GGGSGGGQVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSGGGGSGGGGSGGGGSDIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 116 - protein, 226 aa, synthetic construct
QVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 117 - protein, 215 aa, synthetic construct
DIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 118 - protein, 5 aa, synthetic construct
NYGMN

SEQ ID NO: 119 - protein, 17 aa, synthetic construct
WINTYTGESTYADSFKG

SEQ ID NO: 120 - protein, 7 aa, synthetic construct
FAIKGDY

SEQ ID NO: 121 - protein, 116 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 122 - protein, 16 aa, synthetic construct
RSTKSLLHSNGITYLY

SEQ ID NO: 123 - protein, 7 aa, synthetic construct
QMSNLAS

SEQ ID NO: 124 - protein, 9 aa, synthetic construct
AQNLEIPRT

SEQ ID NO: 125 - protein, 112 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 126 - protein, 250 aa, synthetic construct
GGGSGGGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 127 - protein, 221 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 128 - protein, 219 aa, synthetic construct
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 129 - protein, 5 aa, synthetic construct
EYTIH

SEQ ID NO: 130 - protein, 17 aa, synthetic construct
GINPNNGIPNYNQKFKG

SEQ ID NO: 131 - protein, 15 aa, synthetic construct
RRIAYGYDEGHAMDY

SEQ ID NO: 132 - protein, 124 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 133 - protein, 17 aa, synthetic construct
KSSQSLLYSRNQKNYLA

SEQ ID NO: 134 - protein, 7 aa, synthetic construct
WASTRES

SEQ ID NO: 135 - protein, 9 aa, synthetic construct
QQYFSYPLT

SEQ ID NO: 136 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 137 - protein, 259 aa, synthetic construct
GGGSGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGGSGGGGSGGGGSDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 138 - protein, 229 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 139 - protein, 220 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 140 - protein, 5 aa, synthetic construct
DSWIH

SEQ ID NO: 141 - protein, 17 aa, synthetic construct
WISPYGGSTYYADSVKG

SEQ ID NO: 142 - protein, 9 aa, synthetic construct
RHWPGGFDY

SEQ ID NO: 143 - protein, 118 aa, synthetic construct [sequence omitted]

SEQ ID NO: 144 - protein, 11 aa, synthetic construct
RASQDVSTAVA

SEQ ID NO: 145 - protein, 7 aa, synthetic construct
SASFLYS

SEQ ID NO: 146 - protein, 9 aa, synthetic construct
QQYLYHPAT

SEQ ID NO: 147 - protein, 107 aa, synthetic construct [sequence omitted]

SEQ ID NO: 148 - protein, 247 aa, synthetic construct
GGGSGGGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 149 - protein, 223 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 150 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 5 aa, synthetic construct
SFAMS

SEQ ID NO: 152 - protein, 17 aa, synthetic construct
AISGSGGGTYYADSVKG

SEQ ID NO: 153 - protein, 13 aa, synthetic construct
DKILWFGEPVFDY

SEQ ID NO: 154 - protein, 122 aa, synthetic construct [sequence omitted]

SEQ ID NO: 155 - protein, 11 aa, synthetic construct
RASQSVSSYLA

SEQ ID NO: 156 - protein, 7 aa, synthetic construct
DASNRAT

SEQ ID NO: 157 - protein, 9 aa, synthetic construct
QQRSNWPPT

SEQ ID NO: 158 - protein, 107 aa, synthetic construct [sequence omitted]

SEQ ID NO: 159 - protein, 251 aa, synthetic construct
GGGSGGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 160 - protein, 227 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 161 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 5 aa, synthetic construct
DSNIH

SEQ ID NO: 163 - protein, 17 aa, synthetic construct
YIYPYNGGTDYNQKFKN

SEQ ID NO: 164 - protein, 7 aa, synthetic construct
GNPWLAY

SEQ ID NO: 165 - protein, 116 aa, synthetic construct [sequence omitted]

SEQ ID NO: 166 - protein, 15 aa, synthetic construct
RASESLDNYGIRFLT

SEQ ID NO: 167 - protein, 7 aa, synthetic construct
AASNQGS

SEQ ID NO: 168 - protein, 9 aa, synthetic construct
QQTKEVPWS

SEQ ID NO: 169 - protein, 111 aa, synthetic construct [sequence omitted]

SEQ ID NO: 170 - protein, 249 aa, synthetic construct
GGGSGGGEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 171 - protein, 214 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 172 - protein, 218 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 5 aa, synthetic construct
SYWLH

SEQ ID NO: 174 - protein, 17 aa, synthetic construct
MIDPSNSDTRFNPNFKD

SEQ ID NO: 175 - protein, 10 aa, synthetic construct
YRSYVTPLDY

SEQ ID NO: 176 - protein, 119 aa, synthetic construct [sequence omitted]

SEQ ID NO: 177 - protein, 17 aa, synthetic construct
KSSQSLLYTSSQKNYLA

SEQ ID NO: 178 - protein, 7 aa, synthetic construct
WASTRES

SEQ ID NO: 179 - protein, 9 aa, synthetic construct
QQYYAYPWT

SEQ ID NO: 180 - protein, 113 aa, synthetic construct [sequence omitted]

SEQ ID NO: 181 - protein, 254 aa, synthetic construct
GGGSGGGEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIK

SEQ ID NO: 182 - protein, 224 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 183 - protein, 220 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 5 aa, synthetic construct
NYNMH

SEQ ID NO: 185 - protein, 17 aa, synthetic construct
TIYPGNDDTSYNQKFKD

SEQ ID NO: 186 - protein, 8 aa, synthetic construct
GGYRAMDY

SEQ ID NO: 187 - protein, 117 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 188 - protein, 16 aa, synthetic construct
RSSQSIVYSNGNTYLG

SEQ ID NO: 189 - protein, 7 aa, synthetic construct
KVSNRFS

SEQ ID NO: 190 - protein, 9 aa, synthetic construct
FQGSHVPYT

SEQ ID NO: 191 - protein, 112 aa, synthetic construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 192 - protein, 251 aa, synthetic construct
GGGSGGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGGGSGGGGSGGGGSDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 193 - protein, 222 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 194 - protein, 219 aa, synthetic construct
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 7 aa, synthetic construct
SGDYFWS

SEQ ID NO: 196 - protein, 16 aa, synthetic construct
HIHNSGTTYYNPSLKS

SEQ ID NO: 197 - protein, 12 aa, synthetic construct
DRGGDYYYGMDV

SEQ ID NO: 198 - protein, 122 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSS

SEQ ID NO: 199 - protein, 12 aa, synthetic construct
RASQGISRSYLA

SEQ ID NO: 200 - protein, 7 aa, synthetic construct
GASSRAT

SEQ ID NO: 201 - protein, 9 aa, synthetic construct
QQFGSSPWT

SEQ ID NO: 202 - protein, 108 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 203 - protein, 252 aa, synthetic construct
GGGSGGGQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSGGGGSGGGGSGGGGSEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 204 - protein, 227 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 205 - protein, 215 aa, synthetic construct
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 7 aa, synthetic construct
SGSYYWS

SEQ ID NO: 207 - protein, 16 aa, synthetic construct
YIYYSGSTNYNPSLKS

SEQ ID NO: 208 - protein, 9 aa, synthetic construct
EGKNGAFDI

SEQ ID NO: 209 - protein, 119 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 210 - protein, 11 aa, synthetic construct
RASQSISSYLN

SEQ ID NO: 211 - protein, 7 aa, synthetic construct
AASSLQS

SEQ ID NO: 212 - protein, 9 aa, synthetic construct
QQSYSTPLT

SEQ ID NO: 213 - protein, 107 aa, synthetic construct
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 214 - protein, 248 aa, synthetic construct
GGGSGGGQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 215 - protein, 224 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 216 - protein, 214 aa, synthetic construct
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 5 aa, synthetic construct
GYNMN

SEQ ID NO: 218 - protein, 17 aa, synthetic construct
AIDPYYGGTSYNQKFKG

SEQ ID NO: 219 - protein, 4 aa, synthetic construct
GMEY

SEQ ID NO: 220 - protein, 113 aa, synthetic construct [sequence omitted]

SEQ ID NO: 221 - protein, 16 aa, synthetic construct
RSSQSLVHRNGNTYLH

SEQ ID NO: 222 - protein, 7 aa, synthetic construct
KVSNRFS

SEQ ID NO: 223 - protein, 10 aa, synthetic construct
SQSTHVPPLT

SEQ ID NO: 224 - protein, 113 aa, synthetic construct [sequence omitted]

SEQ ID NO: 225 - protein, 248 aa, synthetic construct
GGGSGGGEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 226 - protein, 218 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 227 - protein, 220 aa, synthetic construct
EIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 216 aa, synthetic construct
DIQLTQPNSVSTSLGSTVKLSCTLSSGNIENNYVHWYQLYEGRSPTTMIYDDDKRPDAVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 229 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 230 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 231 - protein, 470 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 232 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 233 - protein, 466 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 234 - protein, 473 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSGGGGSGGGGSGGGGSDIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 235 - protein, 472 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 236 - protein, 481 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGGSGGGGSGGGGSDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 237 - protein, 469 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 238 - protein, 473 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 239 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 240 - protein, 476 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIK

SEQ ID NO: 241 - protein, 473 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGGGSGGGGSGGGGSDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 242 - protein, 474 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSGGGGSGGGGSGGGGSEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 243 - protein, 470 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 244 - protein, 470 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 245 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 246 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 247 - protein, 471 aa, synthetic construct; identical to SEQ ID NO: 246

SEQ ID NO: 248 - protein, 471 aa, synthetic construct; identical to SEQ ID NO: 245

SEQ ID NO: 249 - protein, 112 aa, synthetic construct
DIQLTQPNSVSTSLGSTVKLSCTLSSGNIENNYVHWYQLYEGRSPTTMIYDDDKRPEGVPDRFSGSIDRSSNSAFLTIHNVAIEDEAIYFCHSYVSSFNVFGGGTKLTVLRQ

SEQ ID NO: 250 - protein, 241 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 251 - protein, 246 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSGGGGSGGGGSGGGGSDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQ

SEQ ID NO: 252 - protein, 241 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 253 - protein, 241 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIK

SEQ ID NO: 254 - protein, 242 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 255 - protein, 242 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 256 - protein, 242 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDATVKLLIYHTSRLHSGVPSRFSGSGSGTDYSLTISNLEQEDIATYFCQQGNTLPYTFGGGTKLEIT

SEQ ID NO: 257 - protein, 242 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPEGTVKLLIYHTSRLHSGVPSRFSGSGSGTDYSLTISNLEQEDIATYFCQQGNTLPYTFGGGTKLEIT

SEQ ID NO: 258 - protein, 241 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 259 - protein, 242 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 260 - protein, 237 aa, synthetic construct
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 261 - protein, 244 aa, synthetic construct
QVQLVQSGSELKKPGASVKVSCKASGYTFTEYGMNVWRQAPGQGLEWMGWINTKSGEATYVEEFKGRFVFSLDTSVSTAYLQISSLKAEDTAVYYCARWDFYDYVDEAMYWGQGTTVTVSSGGGGSGGGGSGGGGSDIQMTQSPSSLSASVGDRVTITCKASQTVSANVAWYQQKPGKAPKLLIYLASYRYRGVPSRFSGSGSGTDFTLTISSLQPEDFATYYCHQYYTYPLFTFGQGTKLEIK

SEQ ID NO: 262 - protein, 243 aa, synthetic construct
EVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGGGSGGGGSGGGGSDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELK

SEQ ID NO: 263 - protein, 252 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGGSGGGGSGGGGSDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 264 - protein, 240 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 265 - protein, 244 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 266 - protein, 242 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 267 - protein, 247 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSGGGGSGGGGSGGGGSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIK

SEQ ID NO: 268 - protein, 244 aa, synthetic construct
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGGGSGGGGSGGGGSDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 269 - protein, 245 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSGGGGSGGGGSGGGGSEIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 270 - protein, 241 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIK

SEQ ID NO: 271 - protein, 241 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSEIVMTQSPATLSVSPGERATLSCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 272 - protein, 7 aa, synthetic construct
SGDYYWT

SEQ ID NO: 273 - protein, 16 aa, synthetic construct
HIYYSGNTNYNPSLKS

SEQ ID NO: 274 - protein, 9 aa, synthetic construct
DRVTGAFDI

SEQ ID NO: 275 - protein, 119 aa, synthetic construct [sequence omitted]

SEQ ID NO: 276 - protein, 11 aa, synthetic construct
QASQDISNYLN

SEQ ID NO: 277 - protein, 7 aa, synthetic construct
DASNLET

SEQ ID NO: 278 - protein, 9 aa, synthetic construct
QHFDHLPLA

SEQ ID NO: 279 - protein, 108 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 280 - protein, 249 aa, synthetic construct
GGGSGGGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 281 - protein, 242 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 282 - protein, 224 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 283 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 5 aa, synthetic construct
SYNMH

SEQ ID NO: 285 - protein, 17 aa, synthetic construct
AIYPGNGDTSYNQKFKG

SEQ ID NO: 286 - protein, 13 aa, synthetic construct
VVYYSNSYWYFDV

SEQ ID NO: 287 - protein, 122 aa, synthetic construct [sequence omitted]

SEQ ID NO: 288 - protein, 10 aa, synthetic construct
RASSSVSYMH

SEQ ID NO: 289 - protein, 7 aa, synthetic construct
APSNLAS

SEQ ID NO: 290 - protein, 9 aa, synthetic construct
QQWSFNPPT

SEQ ID NO: 291 - protein, 107 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 292 - protein, 251 aa, synthetic construct
GGGSGGGEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSGGGGSGGGGSGGGGSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 293 - protein, 244 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSGGGGSGGGGSGGGGSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 294 - protein, 227 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 295 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 5 aa, synthetic construct
DYAMH

SEQ ID NO: 297 - protein, 17 aa, synthetic construct
TISWNSGSIGYADSVKG

SEQ ID NO: 298 - protein, 13 aa, synthetic construct
DIQYGNYYYGMDV

SEQ ID NO: 299 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 300 - protein, 11 aa, synthetic construct
RASQSVSSYLA

SEQ ID NO: 301 - protein, 7 aa, synthetic construct
DASNRAT

SEQ ID NO: 302 - protein, 9 aa, synthetic construct
QQRSNWPIT

SEQ ID NO: 303 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKR

SEQ ID NO: 304 - protein, 252 aa, synthetic construct
GGGSGGGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKR

SEQ ID NO: 305 - protein, 227 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 306 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 307 - protein, 471 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKR

SEQ ID NO: 308 - protein, 474 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKR

SEQ ID NO: 309 - protein, 465 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 310 - protein, 471 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 311 - protein, 469 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPCSRSTSESTAALGCLVKDYFPEPVTVSWNSGALTSGVHTFPAVLQSSGLYSLSSVAIVTVPSSNFGTQTYTCNVDHKPSNTKVDKTVGGGSGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 312 - protein, 472 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKGGGSGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 313 - protein, 477 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKGGGSGGGEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSGGGGSGGGGSGGGGSDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQ

SEQ ID NO: 314 - protein, 472 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKGGGSGGGQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 315 - protein, 468 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKGGGSGGGEVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSGGGGSGGGGSGGGGSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIK

SEQ ID NO: 316 - protein, 466 aa, synthetic construct; identical to SEQ ID NO: 233